SEQUENCE LISTING (34 sequences)

SEQ ID NO: 1 - protein, 1866 aa, Homo sapiens
MDPVGLQLGNKNLWSCLVRLLTKDPEWLNAKMKFFLPNTDLDSRNETLDPEQRVILQLNKLHVQGSDTWQSFIHCVCMQLEVPLDLEVLLLSTFGYDDGFTSQLGAEGKSQPESQLHHGLKRPHQSCGSSPRRKQCKKQQLELAKKYLQLLRTSAQQRYRSQIPGSGQPHAFHQVYVPPILRRATASLDTPEGAIMGDVKVEDGADVSISDLFNTRVNKGPRVTVLLGKAGMGKTTLAHRLCQKWAEGHLNCFQALFLFEFRQLNLITRFLTPSELLFDLYLSPESDHDTVFQYLEKNADQVLLIFDGLDEALQPMGPDGPGPVLTLFSHLCNGTLLPGCRVMATSRPGKLPACLPAEAAMVHMLGFDGPRVEEYVNHFFSAQPSREGALVELQTNGRLRSLCAVPALCQVACLCLHHLLPDHAPGQSVALLPNMTQLYMQMVLALSPPGHLPTSSLLDLGEVALRGLETGKVIFYAKDIAPPLIAFGATHSLLTSFCVCTGPGHQQTGYAFTHLSLQEFLAALHLMASPKVNKDTLTQYVTLHSRWVQRTKARLGLSDHLPTFLAGLASCTCRPFLSHLAQGNEDCVGAKQAAVVQVLKKLATRKLTGPKVVELCHCVDETQEPELASLTAQSLPYQLPFHNFPLTCTDLATLTNILEHREAPIHLDFDGCPLEPHCPEALVGCGQIENLSFKSRKCGDAFAEALSRSLPTMGRLQMLGLAGSKITARGISHLVKALPLCPQLKEVSFRDNQLSDQVVLNIVEVLPHLPRLRKLDLSSNSICVSTLLCLARVAVTCPTVRMLQAREADLIFLLSPPTETTAELQRAPDLQESDGQRKGAQSRSLTLRLQKCQLQVHDAEALIALLQEGPHLEEVDLSGNQLEDEGCRLMAEAASQLHIARKLDLSNNGLSVAGVHCVLRAVSACWTLAELHISLQHKTVIFMFAQEPEEQKGPQERAAFLDSLMLQMPSELPLSSRRMRLTHCGLQEKHLEQLCKALGGSCHLGHLHLDFSGNALGDEGAARLAQLLPGLGALQSLNLSENGLSLDAVLGLVRCFSTLQWLFRLDISFESQHILLRGDKTSRDMWATGSLPDFPAAAKFLGFRQRCIPRSLCLSECPLEPPSLTRLCATLKDCPGPLELQLSCEFLSDQSLETLLDCLPQLPQLSLLQLSQTGLSPKSPFLLANTLSLCPRVKKVDLRSLHHATLHFRSNEEEEGVCCGRFTGCSLSQEHVESLCWLLSKCKDLSQVDLSANLLGDSGLRCLLECLPQVPISGLLDLSHNSISQESALYLLETLPSCPRVREASVNLGSEQSFRIHFSREDQAGKTLRLSECSFRPEHVSRLATGLSKSLQLTELTLTQCCLGQKQLAILLSLVGRPAGLFSLRVQEPWADRARVLSLLEVCAQASGSVTEISISETQQQLCVQLEFPRQEENPEAVALRLAHCDLGAHHSLLVGQLMETCARLQQLSLSQVNLCEDDDASSLLLQSLLLSLSELKTFRLTSSCVSTEGLAHLASGLGHCHHLEELDLSNNQFDEEGTKALMRALEGKWMLKRLDLSHLLLNSSTLALLTHRLSQMTCLQSLRLNRNSIGDVGCCHLSEALRAATSLEELDLSHNQIGDAGVQHLATILPGLPELRKIDLSGNSISSAGGVQLAESLVLCRRLEELMLGCNALGDPTALGLAQELPQHLRVLHLPFSHLGPGGALSLAQALDGSPHLEEISLAENNLAGGVLRFCMELPLLRQIDLVSCKIDNQTAKLLTSSFTSCPALEVILLSWNLLGDEAAAELAQVLPQMGRLKRVDLEKNQITALGAWLLAEGLAQGSSIQVIRLWNNPIPCDMAQHLKSQEPRLDFAFFDNQPQAPWGT

SEQ ID NO: 2 - protein, 785 aa, Homo sapiens
MGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVDATSPAPSTSSTVKTEGAEATPGAQKRKKSTKEKAGPKGSKVSEEQTQPPSPAGAGMSTAMGRSPSPKTSLSAPPNSSSTENPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLTTINCEEGDKLKLTCFELAPKSGNTGELRSVIHSHIKVIKTRKNKKDILNPDSSMETSPDFFF

SEQ ID NO: 3 - protein, 619 aa, Mus musculus
MVNEYKRIVLLRGLECINKHYFSLFKSLLARDLNLERDNQEQYTTIQIANMMEEKFPADSGLGKLIAFCEEVPALRKRAEILKKERSEVTGETSLEKNGQEAGPATPTSTTSHMLASERGETSATQEETSTAQAGTSTAQARTSTAQAGTSTAQKRKIMREEETGVKKSKAAKEPDQPPCCEEPTARCQSPILHSSSSASSNIPSAKNQKSQPQNQNIPRGAVLHSEPLTVMVLTATDPFEYESPEHEVKNMLHATVATVSQYFHVKVFNINLKEKFTKKNFIIISNYFESKGILEINETSSVLEAAPDQMIEVPNSIIRNANASPKICDIQKGTSGAVFYGVFTLHKKTVNRKNTIYEIKDGSGSIEVVGSGKWHNINCKEGDKLHLFCFHLKTIDRQPKLVCGEHSFIKISKRGNVPKEPAKEEDHHHGPKQVMVLKVTEPFTYDLKEDKRMFHATVATETEFFRVKVFDTALKSKFIPRNIIAISDYFGCNGFLEIYRASCVSDVNVNPTMVISNTLRQRANATPKISYLFSQARGTFVSGEYLVNKKTERNKFIYYGIGDDTGKMEVVVYGRLTNVRCEPGSKLRLVCFELTSTEDGWQLRSVRHSYMQVINARK

SEQ ID NO: 4 - protein, 619 aa, Mus musculus
MVNEYKRIVLLAGLECINKHYFSLFKSLLARDLNLERDNQEQYTTIQIANMMEEKFPADSGLGKLIAFCEEVPALRKRAEILKKERSEVTGETSLEKNGQEAGPATPTSTTSHMLASERGETSATQEETSTAQAGTSTAQARTSTAQAGTSTAQKRKIMREEETGVKKSKAAKEPDQPPCCEEPTARCQSPILHSSSSASSNIPSAKNQKSQPQNQNIPRGAVLHSEPLTVMVLTATDPFEYESPEHEVKNMLHATVATVSQYFHVKVFNINLKEKFTKKNFIIISNYFESKGILEINETSSVLEAAPDQMIEVPNSIIRNANASPKICDIQKGTSGAVFYGVFTLHKKTVNRKNTIYEIKDGSGSIEVVGSGKWHNINCKEGDKLHLFCFHLKTIDRQPKLVCGEHSFIKISKRGNVPKEPAKEEDHHHGPKQVMVLKVTEPFTYDLKEDKRMFHATVATETEFFRVKVFDTALKSKFIPRNIIAISDYFGCNGFLEIYRASCVSDVNVNPTMVISNTLRQRANATPKISYLFSQARGTFVSGEYLVNKKTERNKFIYYGIGDDTGKMEVVVYGRLTNVRCEPGSKLRLVCFELTSTEDGWQLRSVRHSYMQVINARK

SEQ ID NO: 5 - protein, 619 aa, Mus musculus
MVNEYKRIVLLRGLECINKHYFSLFKSLLARDLNLERDNQEQYTTIQIANMMEEKFPADSGLGKLIAFCEEVPALRKRAEILKKERSEVTGETSLEKNGQEAGPATPTSTTSHMLASERGETSATQEETSTAQAGTSTAQARTSTAQAGTSTAQKRKIMREEETGVKKSKAAKEPDQPPCCEEPTARCQSPILHSSSSASSNIPSAKNQKSQPQNQNIPRGAVLHSEPLTVMVLTATDPFEYESPEHEVKNMLHATVATVSQYFHVKVFNINLKEKFTKKNFIIISNYFESKGILEINETSSVLEAAPDQMIEVPNSIIRNANASPKICDIQKGTSGAVFYGVFTLHKKTVNRKNTIYEIKDGSGSIEVVGSGKWHNINCKEGDKLHLFCFHLKTIDRQPKLVCGEHSFIKISKRGNVPKEPAKEEDHHHGPKQVMVLKVTEPFTYDLKEDKRMFHATVATETEFFRVKVFDTALKSKFIPRNIIAISDYFGCNGFLEIYRASCVSDVNVNPTMVISNTLRQRANATPKISYLFSQAAGTFVSGEYLVNKKTERNKFIYYGIGDDTGKMEVVVYGRLTNVRCEPGSKLRLVCFELTSTEDGWQLRSVRHSYMQVINARK

SEQ ID NO: 6 - protein, 619 aa, Mus musculus
MVNEYKRIVLLAGLECINKHYFSLFKSLLARDLNLERDNQEQYTTIQIANMMEEKFPADSGLGKLIAFCEEVPALRKRAEILKKERSEVTGETSLEKNGQEAGPATPTSTTSHMLASERGETSATQEETSTAQAGTSTAQARTSTAQAGTSTAQKRKIMREEETGVKKSKAAKEPDQPPCCEEPTARCQSPILHSSSSASSNIPSAKNQKSQPQNQNIPRGAVLHSEPLTVMVLTATDPFEYESPEHEVKNMLHATVATVSQYFHVKVFNINLKEKFTKKNFIIISNYFESKGILEINETSSVLEAAPDQMIEVPNSIIRNANASPKICDIQKGTSGAVFYGVFTLHKKTVNRKNTIYEIKDGSGSIEVVGSGKWHNINCKEGDKLHLFCFHLKTIDRQPKLVCGEHSFIKISKRGNVPKEPAKEEDHHHGPKQVMVLKVTEPFTYDLKEDKRMFHATVATETEFFRVKVFDTALKSKFIPRNIIAISDYFGCNGFLEIYRASCVSDVNVNPTMVISNTLRQRANATPKISYLFSQAAGTFVSGEYLVNKKTERNKFIYYGIGDDTGKMEVVVYGRLTNVRCEPGSKLRLVCFELTSTEDGWQLRSVRHSYMQVINARK

SEQ ID NO: 7 - DNA, 69 bp, Artificial Sequence
  Description of Artificial Sequence Synthetic oligonucleotide
ccatcagaaagaggtttaatatttttgtgagaccatcggggccgcgcctcccccgcgaggccgccggcg

SEQ ID NO: 8 - DNA, 19 bp, Artificial Sequence
  Description of Artificial Sequence Synthetic primer
tgtggccctccgtgaaatc

SEQ ID NO: 9 - DNA, 23 bp, Artificial Sequence
  Description of Artificial Sequence Synthetic primer
ggcataattgttacacgtttggc

SEQ ID NO: 10 - DNA, 21 bp, Artificial Sequence
  Description of Artificial Sequence Synthetic primer
ccaagtgctgccgtcattttc

SEQ ID NO: 11 - DNA, 21 bp, Artificial Sequence
  Description of Artificial Sequence Synthetic primer
ggctcgcagggatgatttcaa

SEQ ID NO: 12 - DNA, 20 bp, Artificial Sequence
  Description of Artificial Sequence Synthetic primer
ctcaccatatggctcggaca

SEQ ID NO: 13 - DNA, 20 bp, Artificial Sequence
  Description of Artificial Sequence Synthetic primer
cttctctgggttggcacaca

SEQ ID NO: 14 - DNA, 21 bp, Artificial Sequence
  Description of Artificial Sequence Synthetic primer
cagctccaagaaaggacgaac

SEQ ID NO: 15 - DNA, 21 bp, Artificial Sequence
  Description of Artificial Sequence Synthetic primer
ggcagtgtaactcttctgcat

SEQ ID NO: 16 - DNA, 21 bp, Artificial Sequence
  Description of Artificial Sequence Synthetic primer
gtgccaaacgtccttttcaga

SEQ ID NO: 17 - DNA, 21 bp, Artificial Sequence
  Description of Artificial Sequence Synthetic primer
agtgaggagtaagccatgctc

SEQ ID NO: 18 - DNA, 21 bp, Artificial Sequence
  Description of Artificial Sequence Synthetic primer
ggacttgccttgttccgagag

SEQ ID NO: 19 - DNA, 22 bp, Artificial Sequence
  Description of Artificial Sequence Synthetic primer
gctgccacataactgatagcga

SEQ ID NO: 20 - DNA, 21 bp, Artificial Sequence
  Description of Artificial Sequence Synthetic primer
ttctggtgcttgtctcactga

SEQ ID NO: 21 - DNA, 22 bp, Artificial Sequence
  Description of Artificial Sequence Synthetic primer
cagtatgttcggcttcccattc

SEQ ID NO: 22 - DNA, 22 bp, Artificial Sequence
  Description of Artificial Sequence Synthetic primer
catgaaccgagatggctctagt

SEQ ID NO: 23 - DNA, 23 bp, Artificial Sequence
  Description of Artificial Sequence Synthetic primer
tcatcgtagaattttggcagctc

SEQ ID NO: 24 - DNA, 19 bp, Artificial Sequence
  Description of Artificial Sequence Synthetic primer
gagcaaggcggctaaggaa

SEQ ID NO: 25 - DNA, 21 bp, Artificial Sequence
  Description of Artificial Sequence Synthetic primer
gctgtggagtattggtgactg

SEQ ID NO: 26 - DNA, 21 bp, Artificial Sequence
  Description of Artificial Sequence Synthetic primer
ctgaattgcgtccccgaaata

SEQ ID NO: 27 - DNA, 21 bp, Artificial Sequence
  Description of Artificial Sequence Synthetic primer
aggttcctgaatgaactccct

SEQ ID NO: 28 - DNA, 21 bp, Artificial Sequence
  Description of Artificial Sequence Synthetic primer
cttgctgtgctggattcaagc

SEQ ID NO: 29 - DNA, 22 bp, Artificial Sequence
  Description of Artificial Sequence Synthetic primer
caactgtggtaagaagggagtg

SEQ ID NO: 30 - DNA, 20 bp, Artificial Sequence
  Description of Artificial Sequence Synthetic primer
tggggcctaagcctatgtct

SEQ ID NO: 31 - DNA, 20 bp, Artificial Sequence
  Description of Artificial Sequence Synthetic primer
ctcccaagggtggctttagg

SEQ ID NO: 32 - DNA, 21 bp, Artificial Sequence
  Description of Artificial Sequence Synthetic primer
ctgccgatactgaacctgagc

SEQ ID NO: 33 - DNA, 19 bp, Artificial Sequence
  Description of Artificial Sequence Synthetic primer
gcggtcaaaatcgcactcc

SEQ ID NO: 34 - protein, 6 aa, Artificial Sequence
  Description of Artificial Sequence Synthetic 6xHis tag
HHHHHH

(19) United States
(12) Patent Application Publication
(10) Pub. No.: US 2023/0301998 A1
(43) Pub. Date: Sep. 28, 2023

(54) METHODS AND COMPOSITIONS FOR INDUCTION OF ANTITUMOR IMMUNITY

(71) Applicant: Sanford Burnham Prebys Medical Discovery Institute, La Jolla, CA (US)

(72) Inventors: Ze'ev A. RONAI, Fallbrook, CA (US); Hyungsoo KIM, San Diego, CA (US)

(21) Appl. No.: 17/997,757

(22) PCT Filed: May 3, 2021
(86) PCT No.: PCT/US21/30478

§ 371 Date: Nov. 2, 2022

Related U.S. Application Data
(60) Provisional application No. 63/019,914, filed on May 4, 2020.

(51) Int. Cl. A61K 31/506; C07K 16/28; A61P 35/00; C12N 9/22

(52) U.S. Cl. CPC A61K 31/506; C07K 16/2818; C07K 16/2827; A61P 35/00; C12N 9/22

(57) ABSTRACT

Provided herein are methods and compositions for diagnosing, treating, or ameliorating symptoms of cancer, including melanoma, with inhibitors of PRMTS and immune response regulators in combination with checkpoint inhibitor therapy.

### CROSS-REFERENCE

This application claims the benefit of U.S. Provisional Application No. 63/019,914, filed on May 4, 2020, which application is incorporated herein by reference in its entirety.

### STATEMENT AS TO FEDERALLY SPONSORED RESEARCH

This invention was made with government support under CA 19746 and CA 198468 awarded by the National Institutes of Health. The government has certain rights in the invention.

### FIELD

The disclosure is generally in the field of cancer and cancer treatment and specifically in the area of diagnosis, prognosis, treatment, monitoring treatment, and selecting treatment of cancer.

### BACKGROUND

All publications herein are incorporated by reference to the same extent as if each individual publication or patent application was specifically and individually indicated to be incorporated by reference. The following description includes information that may be useful in understanding the present disclosure. It is not an admission that any of the information provided herein is prior art or relevant to the presently claimed subject matter, or that any publication specifically or implicitly referenced is prior art.

Protein arginine methyltransferase 5 (PRMT5) controls diverse cellular processes implicated in cancer development and progression. PRMT5 catalyzes monomethylation and symmetric dimethylation of arginine (Arg, R) residues on histones and non-histone proteins, thereby regulating diverse processes related to oncogenesis, including transcription, RNA splicing, translation and the DNA damage response (Stoba et al., CMLS 2015; 72(11): 2041-59; Yang et al., Nature Reviews Cancer 2013; 13(1): 37-50). Specific examples include lymphomagenesis, where PRMT5 is implicated in controlling pre-mRNA splicing; lung cancer in which PRMT5 is linked to control of metastasis; and glioblastoma, where PRMT5 is implicated in removal of introns retained in proliferation genes (Koh et al., Nature 2015; 523(7558): 96-100; Chen et al., Oncogene 2017, 36(3): 373-86; Braun et al., Cancer Cell 2017; 32(4): 411-26). The role of PRMT5 activity in cancer is highlighted in ˜20-40% of tumors that harbor deletion of MTAP (methylthioadenosine phosphorylase) gene, which is often co-deleted with CDKN2A. Interestingly, MTAP-deleted tumors are relatively more sensitive to PRMT5 inhibition (Kryukov et al., Science 2016; 351(6278): 1214-8; Mavrakis et al., Science 20161351 (6278): 1208-13; Marjon et al., Cell Reports 2016; 15(3): 574-87; Tamiya et al., The Journal of clinical investigation 2018;128(1):517-30), each incorporated by reference in its entirety). Several adaptor proteins are reportedly important for PRMT5 activity and substrate selectivity. Among them, WDR77 functions in histone methylation and concomitant transcriptional repression by PRMT5. The adaptor pCln/CLNS1A impacts PRMT5 -dependent SnRNAP (small nuclear ribonucleoproteins) methylation and subsequent splicing, while the adaptor SHARPIN contributes to PRMT5 dependent methylation of SKI, resulting in SOX10 transcriptional activation (Burgos et al. J. Biol. Chem. 290, 9674-9689 (2015); Meister et al. Curr. Biol. 11, 1990-1994 (2001), each incorporated by reference in its entirety) Thus, PRMT5 , or factors required for its activity, emerge as promising targets for therapy, especially in MTAP deleted tumors. Small molecules inhibitors of PRMT5 have been developed. A SAM uncompetitive PRMT5 inhibitor (GSK3326595) that was reported to activate p53-MDM4 axis through control of cellular splicing (Gerhart et al., Scientific Reports 2018; 8(1): 9711; Almine et al., Nature communications 2017;8:14392), and a SAM competitive PRMT5 inhibitor (JNJ-64619178) are being evaluated in phase I clinical trial for non-Hodgkin's lymphoma and solid tumors (NCT02783300, NCT03573310). Notably, PRMT5 inhibition was also shown to affect embryonic development and hematopoiesis, suggesting that further studies are required to assess mechanisms underlying response to PRMS inhibitors in different cell types (Tee et al., Genes & development 2010;24(24):2772-7; Liu et al., The Journal of clinical investigation 2015;125(9):3532-44).

Better understanding of immune checkpoint regulatory pathways is expected to increase the rate of success and efficacy of immune checkpoint therapy (ICT). Unresponsiveness and resistance tumors to immune checkpoint therapy, namely cold tumors, present one of the major obstacles for effective immune checkpoint therapy. At present, only a subset of tumor types benefits from ICT, while a notable percentage of patients either fails to respond or acquires resistance to ICT (10-44% objective response rate following Ipilimumab, Nivolumab, or Pembrolizumab treatment in advanced melanoma) (Hodi et al. The New England journal of medicine 2010;363(8):711-23; Postow et al., The New England journal of medicine 2015;372(21):2006-17; Larkin et al., The New England journal of medicine 2015;373(1):23-34; Schachter et al, Lancet 2017;390(10105):1853¬62). Among tumor intrinsic mechanisms are infiltration and activation of immune cells, especially CD8 T cells, as well as loss of tumor antigenicity. Activation of oncogenic Wnt/beta-catenin signaling or loss of tumor suppressor PTEN expression hampers CD8 T cell tumor infiltration of tumors and confers resistance to ICT (Spranger et al., Nature 2015;523(7559):231-5 ; Peng et al., Cancer discovery 2016;6(2):202-16). Expression of chemokines (such as CXCL9 and CXCL10) or upregulation of the type I interferon response is also regulated by epigenetic factors, including EZH2 (Histone-lysine N-methyltransferase) and LSD1 (Lysine-specific histone demethylase), both of which alter CD8 T cell recruitment to tumors (Peng et al., Nature 2015;527(7577):249-53; Sheng et al., Cell 2018;174(3):549-63). Loss of antigen presentation, a mechanism underlying tumor intrinsic immune evasion, is associated with tumor resistance to ICT. Homozygous deletion of B2M (beta-2-microglobulin), a beta subunit for all HLA class I complexes, impairs antigen processing and presentation by tumor cells, contributing to the resistance of to ICT in melanoma and lung cancer (Gettinger et al; Cancer Discovery 2017; 7(12): 1420-35; Sade-Feldman et al., Nature Communications 2017; 8(1): 1136). Not intended to be bound by any theory, the control of tumor intrinsic immune suppression, in part through altering interferon response, chemokine production and antigen presentation, may constitute novel therapeutic targets to overcome resistance to ICT. Thus, there is a need for identifying and characterizing genes involved in tumor-intrinsic immune response, particularly in antitumor immune response in melanoma. Provided herein are compositions and methods that relate to unveiled unrecognized functions on immune suppressive phenotype which defines cold tumors and thus provide an important strategy to improve the effectiveness of the current immunotherapy, with and upon combination with methyltransferase inhibition.

### SUMMARY

Provided herein are methods and compositions for diagnosing, treatment, monitoring treatment, and selecting treatment of cancer. The disclosed methods and compositions are particularly suited for treatment of melanoma.

In some aspects, provided herein are pharmaceutical compositions for the treatment of cancer. In some embodiments, the pharmaceutical composition comprises a therapeutically effective amount of PRMT5 inhibitor and a therapeutically effective amount of an immunotherapeutic agent, wherein the PRMT5 inhibitor is capable of decreasing expression or activity of a PRMT5 protein. In some embodiments, the PRMT5 inhibitor can decrease expression of a PRMT5 gene that encodes the PRMT5 protein. In some embodiments, the immunotherapeutic agent is a checkpoint inhibitor. In some embodiments, the immunotherapeutic agent is a PD-1 inhibitor, a PD-L1 inhibitor, or a CTLA-4 inhibitor. In some embodiments, the immunotherapeutic agent is selected from the group consisting of pembrolizumab, nivolumab, cemiplimab, atezolizumab, avelumab, durvalumab, and ipilimumab. In some embodiments, the immunotherapeutic agent is involved in or regulated by KRAS signaling, IL2/STAT5 signaling, inflammatory response, TNFa signaling, IL6/JAK/STAT3 signaling, androgen response, TGF beta signaling, apoptosis, interferon alpha response, interferon gamma response, UV response, allograft rejection, or Thl cell and Th2 cell activation. In some embodiments, the immunotherapeutic agent is an interferon, a chemokine, a lymphokine, an interleukin, or a monokine. In some embodiments, the pharmaceutical composition further comprises an effector protein or a polynucleotide encoding the effector protein, wherein the effector protein is selected from the group consisting of MYH9, MYH10, FASN, GSTP, VIM, CLTC, HSPA8, PKM, P4HB, TUBB, SLC25A13, FLNA, PFKFB2, HSPD1, HSPA5, XRCC5, XRCC6, RNF31, MYL12B, MYL12A, HSPA9, GAPDH, ATP5B, HNRNPU, PFKFB3, RBM10, GSN, PRPF31, DYNC1H1, IFI16, IFI204, PARP1, PMEL, PNKP, SLC25A4, PDIA6, and RBCK1, APEX1, CHD8, GDAP1, GPHN, IPO4, MAP3K9, NLRC5, OXA1L, and RHOF. In some embodiments, the effector protein is RFN31, IFI16, IFI204, NLRC5, or RBCK1. In some embodiments, the effector protein shares at least 90% identity to SEQ ID NO: 1. In some embodiments, the effector protein shares at least 90% identity to SEQ ID NO: 2. In some embodiments, the effector protein shares at least 90% identity to SEQ ID NO: 3. In some embodiments, the effector protein comprises a mutation that affects methylation of the effector protein. In some embodiments, the mutation is an R to X mutation, wherein X is any amino acid residue. In some embodiments, the mutation is an R to A mutation. In some embodiments, the mutation is an R to C mutation. In some embodiments, the mutation is in amino acid residue 12 of SEQ ID NO: 3 or in a corresponding amino acid residue in a homolog thereof. In some embodiments, the effector protein comprises a second mutation that affects methylation of the effector protein. In some embodiments, the second mutation is an R to X mutation, wherein X is any amino acid residue. In some embodiments, the second mutation is an R to A mutation. In some embodiments, the second mutation is an R to C mutation. In some embodiments, the second mutation is in amino acid residue 538 of SEQ ID NO: 3 or in a corresponding amino acid residue in a homolog thereof. In some embodiments, the effector protein comprises SEQ ID NO:4. In some embodiments, wherein the effector protein comprises SEQ ID NO: 5. In some embodiments, the effector protein comprises SEQ ID NO: 6. In some embodiments, the effector protein or the polynucleotide encoding the effector protein is encoded by a vector. In some embodiments, the vector is an AAV vector, a lentivirus vector, an adenovirus vector, a retrovirus vector, or a herpes simplex virus (HSV-1) vector. In some embodiments, the PRMT5 inhibitor is a small molecule. In some embodiments, the PRMT5 inhibitor is a siRNA. In some embodiments, the PRMT5 inhibitor is a transcription activator like effector nuclease (TALEN). In some embodiments, the PRMT5 inhibitor is a CRISPR-Cas9 complex comprising a Cas9 nuclease and a guide RNA, wherein the guide RNA hybridizes with a target sequence within the PRMT5 gene. In some embodiments, the pharmaceutical composition provided herein further comprises a pharmaceutically acceptable carrier. In some embodiments, the pharmaceutically acceptable carrier is a nanoparticle, a liposome, or a carbon nanotube.

Further provided herein are methods for suppressing tumor growth. The method for suppressing tumor growth may comprise administering to a subject comprising a tumor the any one of the pharmaceutical composition provided herein. In some embodiments, administration of the pharmaceutical composition provided herein results in expression of the PRMT5 gene in the subject is reduced by at least 10%, 20%, 30%, 40%, 50%, 60%, 70%, 80%, 90%, 99%, or 100%. In some embodiments, the tumor is reduced by at least 10%, 20%, 30%, 40%, 50%, 60%, 70%, 80%, 90%, 99%, or 100% in size. In some embodiments, the pharmaceutical composition is administered administered orally, intravenously, intrathecally, subcutaneously, intramuscularly, sublingually, rectally, cutaneously, or transdermally.

In one aspect, provided herein are pharmaceutical compositions comprising a therapeutically effective amount of at least one of (i) an interferon gamma inducible (IFI) protein or a polynucleotide encoding the IFI protein, and (ii) a NLRCS protein or a polynucleotide encoding the NLRCS protein. In some embodiments, the NLRCS protein comprises a polypeptide having at least 90% identity to SEQ ID NO: 1. In some embodiments, the IFI protein comprises a polypeptide having at least 90% identity to SEQ ID NO: 2. In some embodiments, the IFI protein comprises a polypeptide having at least 90% identity to SEQ ID NO: 3. In some embodiments, the IFI protein comprises a mutation that affects methylation of the IFI protein. In some embodiments, the mutation is an R to X mutation, wherein X is any amino acid residue. In some embodiments, the mutation is an R to A mutation. In some embodiments, the mutation is an R to C mutation. In some embodiments, the mutation is in amino acid residue 12 of SEQ ID NO: 3 or in a corresponding amino acid residue in a homolog thereof. In some embodiments, the IFI protein comprises a second mutation that affects methylation of the IFI protein. In some embodiments, the second mutation is an R to X mutation, wherein X is any amino acid residue. In some embodiments, the second mutation is an R to A mutation. In some embodiments, the second mutation is an R to C mutation. In some embodiments, the second mutation is in an amino acid residue 538 of SEQ ID NO: 3 or in a corresponding amino acid residue in a homolog thereof. In some embodiments, the IFI protein comprises SEQ ID NO: 4. In some embodiments, the IFI protein comprises SEQ ID NO: 5. In some embodiments, the IFI protein comprises SEQ ID NO: 6. In some embodiments, the IFI protein or the polynucleotide encoding the IFI protein is encoded by a first vector. In some embodiments, the NLRC5 protein or the polynucleotide encoding the NLRC5 protein is encoded by a second vector. In some embodiments, the first vector and the second vector are a same vector. In some embodiments, the first vector is an AAV vector, a lentivirus vector, an adenovirus vector, a retrovirus vector, or a herpes simplex virus (HSV-1) vector. In some embodiments, the second vector is vector is an AAV vector, a lentivirus vector, an adenovirus vector, a retrovirus vector, or a herpes simplex virus (HSV-1) vector.

Further provided herein are pharmaceutical compositions comprising a therapeutically effective amount of a fusion protein or a polynucleotide encoding the fusion protein, wherein the fusion protein comprises a nucleic acid recognition domain and a nucleobase modifying domain, wherein the nucleic acid recognition domain recognizes a target sequence in a nucleic acid that encodes an Interferon gamma inducible (IFI) protein. In some embodiments, the nucleic acid recognition domain is a zinc finger domain. In some embodiments, the DNA recognition domain is a Transcription activator like effector (TALE) protein domain. In some embodiments, the DNA recognition domain comprises a CRISPR-Cas protein domain. In some embodiments, the pharmaceutical composition provided herein further comprises a guide RNA, wherein the guide RNA binds the target sequence and directs the nucleic acid recognition domain to the target sequence. In some embodiments, the CRISPR-Cas protein domain is a Cas9 domain. In some embodiments, the Cas9 domain does not have nuclease activity. In some embodiments, the Cas9 domain is a nickase domain. In some embodiments, the nucleobase modifying domain comprises a deaminase domain. In some embodiments, the deaminase domain is a cytidine deaminase domain. In some embodiments, the nucleobase modifying domain is capable of introducing a single nucleotide substitution to the nucleic acid encoding the IFI protein. In some embodiments, the single nucleotide substitution affects methylation of the IFI protein. In some embodiments, the single nucleotide substitution is a C to T substitution, and wherein the C to T substitution results in an R to C mutation in the IFI protein. In some embodiments, the IFI protein comprises a polypeptide that is at least 90% identical to SEQ ID NO: 2. In some embodiments, the IFI protein comprises a polynucleotide that is at least 90% identical to SEQ ID NO: 3. In some embodiments, the R to C mutation is in amino acid residue 12 of SEQ ID NO: 3 or in a corresponding amino acid residue in a homolog thereof. In some embodiments, the mutation is in amino acid 538 of SEQ ID NO: 3 or in a corresponding amino acid residue in a homolog thereof. In some embodiments, the nucleic acid that encodes the IFI protein comprises SEQ ID NO: 7. In some embodiments, the polynucleotide encoding the fusion protein is encoded by a vector. In some embodiments, the vector is an AAV vector, a lentivirus vector, an adenovirus vector, a retrovirus vector, or a herpes simplex virus (HSV-1) vector. In some embodiments, the pharmaceutical composition provided herein further comprises a NLRCS protein or a polynucleotide encoding the NLRCS protein. In some embodiments, the NLRCS protein comprises a polypeptide having at least 90% identity to SEQ ID NO: 1.In some embodiments, the pharmaceutical composition provided herein further comprises a pharmaceutically acceptable carrier. In some embodiments, the pharmaceutically acceptable carrier is a nanoparticle, a liposome, or a carbon nanotube.

Also provided herein are methods for treatment of cancer. In one aspect, provided herein are methods for suppressing tumor growth, comprising administering to a subject comprising a tumor the pharmaceutical composition of any one of the pharmaceutical compositions herein provided. In some embodiments, the pharmaceutical composition is administered orally, intravenously, intrathecally, subcutaneously, intramuscularly, sublingually, rectally, cutaneously, or transdermally. In some embodiments, the tumor is a solid tumor. In some embodiments, tumor is a melanoma. In some embodiments, the tumor is reduced by at least 10%, 20%, 30%, 40%, 50%, 60%, 70%, 80%, 90%, 99%, or 100% in size by the treatment.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent application contains at least one drawing executed in color. Copies of this patent or patent application with color drawing(s) will be provided by the Office upon request and payment of the necessary fee.

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-E depict SHARPIN expression associated with immune genes and pathways. FIGS. 1A and 1B depict overall survival rate of melanoma patients adjusted to SHARPIN expression levels. FIGS. 1C and 1D depict IPA (FIG. 1C) and GSEA (FIG. 1D) in low MTAP/low SHARPIN versus low MTAP/high SHARPIN groups. FIG. 1E depicts enriched gene ontology (GO) gene set from GSEA of TCGA data for tumors harboring low or high PRMT5 expression. FIG. 1F depicts a heatmap of 3528 differentially expressed genes (−0.5 ≥Log2 (fold change)≥0.5, FDR≤0.01) between PRMT5 low (n=100, grey bar) and high (n=100, yellow bar) TCGA-SKCM samples. The columns represent samples and rows represent genes. The normalized expression levels (FPKM+0.1) for each gene was row normalized to z-scores and k-means clustered (K=7). Genes associated with immune signature are shown.

FIGS. 2A-C depict enriched immune gene signature shown with melanoma specimens with low PRMT5 expression. FIG. 2A depicts PRMT5 expression in metastatic melanoma specimens based on TCGA datasets. Inset shows comparison between low (blue box on left) and high (red box on right) PRMT5 expression cohorts (n=368). FIG. 2B depicts top-ranked pathways predicted using the Ingenuity Pathway Analysis (IPA) based on differentially-expressed genes (DEGs) in specimens exhibiting either low or high PRMT5 expression. Dark blue bars indicate pathways likely inhibited in the PRMT5-high group. FIG. 2C depicts representative immune gene sets enriched in GSEA of DEGs from melanoma specimens with low or high PRMT5. The top 14 genes for each gene set are shown in respective heatmaps.

FIG. 3 depicts enrichment of immune-associated genes in PRMT5 low melanomas. Analysis of an independent melanoma cohort (GSE78220) for gene sets associated with low versus high PRMT5 expression. Analysis identified enrichment of genes associated with allograft rejection (left) and the interferon gamma response (right) in low PRMT5 patient specimens.

FIGS. 4A-E depicts PRMT5 expression or activity linked with immune associated gene sets. FIG. 4A depicts relative expression (RNAseq v2) of indicated protein methyl transferases (PRMTs) in human melanoma specimens (TCGA). FIG. 4B depicts Pearson's correlation-based analysis of PRMTs co-expression in human melanoma specimen (TCGA). FIG. 4C depicts survival of melanoma patients adjusted to the relative expression of different PRMTs (TCGA, n=428) “z score” represents normalized expression calculated by cbioportal. FIG. 4D depicts differentially-expressed genes (DEGs) in melanoma patient specimens with low PRMT5 expression; analysis was performed with GSEA using the hallmark gene sets. Asterisks appear above the PRMT5 column. NES (normalized enrichment score) and FDR-q (false discovery rate q value) are presented. FIG. 4E depicts DEGs from melanoma patient specimens harboring low or high levels of MTAP expression (TCGA) analyzed with GSEA using the hallmark gene sets.

FIGS. 5A-L depict attenuation of melanoma growth following PRMT5 inhibition. FIG. 5A depicts western blot analysis showing PRMT5 expression (upper) and activity (middle) in protein extracts prepared from B16 murine melanoma cells transduced with scrambled or PRMT5-specific hairpin shRNAs (shPRMT5-1 or shPRMT5-2) and probed with indicated antibodies. Beta-actin served as loading control (lower). Hereafter, “S.exp” and “L.exp” represent short and long exposure, respectively. “SDME-RG” indicates anti-symmetric dimethyl arginine antibody (Cell Signaling). FIG. 5B depicts growth in culture of B16 cells stably expressing shPRMT5 or Scr (scrambled) control. FIG. 5C depicts volume of control and Prmt5 KD B16 tumors (Scr; n=8, shPRMT5-1; n=8, shPRMT5-2; n=7) grafted (s.c., 0.2 million cells) into immunocompetent C57BL/6 mice and measured at indicated time points. FIG. 5D depicts volume of control and Prmt5 KD B16 tumors (Scr; n=5, shPRMT5; n=6) grafted (s.c., 0.2 million cells) into immunocompromised NSG mice and measured at indicated time points. FIG. 5E depicts western blot analysis of PRMT5 expression (upper) and activity (middle) in extracts of tumor cells cultured from indicated tumor pools (Scr-pool, cells from 5 Scrambled-KD tumors; shPRMT5 pools 1 and 2, cells from 3 shPRMT5-KD tumors each). Beta-actin served as loading control (lower). FIG. 5F depicts control or shPRMT5-KD tumor cells isolated and pooled from tumors grown in NSG mice were re-grafted into syngeneic immunocompetent mice (Scr; n=5, shPRMT5; n=6) and assessed at indicated time points. FIG. 5G depicts western blot analysis of PRMT5 expression (upper) and activity (lower) in tumors generated as in FIG. 5F, using indicated antibodies. GAPDH served as loading control (middle). FIG. 511 depicts western blot analysis of PRMT5 expression and activity in YUMMER1.7 cells expressing indicated expression vectors. GAPDH served as a loading control. FIG. 5I depicts growth of YUMMER1.7 cells in culture following transfection with control (EV+EV) or PRMT5+WDR77 constructs. (Protein analysis is shown in FIG. 511.) FIGS. 5J and 5K depicts volume of control and PRMT5+WDR77-overexpressing YUMMER1.7 cell tumors (Scr; n=8, PRMT5+WDR77; n=8) grafted (s.c., 0.4 million cells) into C57BL6 (J) or NSG (K) mice and measured at indicated time points. FIG. 5L depicts western blot analysis of PRMT5 expression and activity in tumors generated as in FIG. 5K, using indicated antibodies. Beta-actin served as loading control. Data are presented as means ±s.d.*p<0.05; ** p<0.01; ***p<0.001; **** p<0.0001; “ns” not significant.

FIGS. 6A-M depict PRMT5 control of tumor growth and immune cell infiltration. C57BL/6 mice were inoculated with B16F10 cells transduced with scrambled (Scr) or Prmt5-specific shRNAs (shPRMT5-1, shPRMT5-2). Four lysates from 4 different tumors of each group were analyzed. FIG. 6A shows tumor weight at time of collection (17 days). FIG. 6B depicts western blot analysis of PRMT5 expression (upper panel) and activity (middle panel) with indicated antibodies. GAPDH(lower panel) served as loading control. FIG. 6C depicts weight of B16F10 tumors transduced with scrambled (Scr) or Prmt5-specific (shPRMT5) shRNAs in NSG mice, as assessed at experiment's end (day 19). FIG. 6D depicts weight of B16F10 tumors described in FIG. 6C re-grafted into C57BL/6 mice at the end of experiment (day 17). FIG. 6E depicts YUMM1.7 cells transduced with empty vector (EV) or doxycyclin (Dox)-inducible shPRMT5 were grafted into C57BL/6 mice and 14 days later animals were administered Dox (black arrow). Tumor growth was assessed at indicated time points. FIG. 6F depicts western blot analysis ofPRMT5 protein and activity (based on methyl-Arg) in YUMM1.7 cells grafted to mice in FIG. 6F. Dox (1 μg/ml) was added for 48 hr to induce PRMT5-KD. GAPDH served as loading control. FIG. 6G depicts growth in culture of YUMM1.7 cells used to generate tumors in FIG. 6F. FIG. 6H depicts western blot analysis of PRMT5, WDR77 and methyl-Arg in indicated YUMMER1.7 tumors. GAPDH served as loading control. One lysate from a tumor expressing EV+EV and 5 lysates from tumors expressing PRMT5+WDR77were analyzed. FIG. 6I depicts weight of B16F10 tumors transduced with scrambled or shPRMT5 grown inC57BL/6 mice. FIG. 6J depicts PRMT5 protein levels and activity (based on methyl-Arg) in B16F10 tumors in FIG. 6I. Four lysates from Scr-KD and 3 lysates from PRMT5-KD tumors were analyzed. GAPDH served as loading control. FIG. 6K depicts representative plots showing the strategy used for gating immune cell populations from cells collected from tumor tissues. P1, P2, P3 and P4 indicate hierarchy of gating. Boxes with different colors indicate a batch of staining with respective antibody cocktail. FIGS. 6L and 6M depict C57BL/6 mice (n=8)were treated with control IgG, anti-NK1.1 (FIG. 6L) or anti-CD8+ (FIG. 6M) antibodies injected 5 times (200 μg/mouse per injection) every three days starting one day prior to tumor cell inoculation. Mouse peripheral blood was collected at day 8 and assessed by flow cytometry for efficient depletion of NK1.1 (FIG. 6L) or CD8+ (FIG. 6M) cells.

FIGS. 7A-H depict invasion by tumor infiltrating leukocytes (TILs) decreased by PRMT5 expression. FIGS. 7A and 7B depict immune phenotyping performed using flow cytometry using the indicated cell surface markers on control and shPRMT5-transduced B16 tumors, collected at day 17. Ratio of abundance was calculated by dividing number of activated CD8 T cells (CD44hiCD8+) by that of regulatory T cells (CD4+FOXP3+). FIG. 7C depicts infiltration of CD4+ and CD8+ immune cells into B16 tumors 12 days after grafting into C57BL/6 mice, as evaluated by immunohistochemistry (left). Quantification of infiltrated immune cells in Scr-KD (n=5) and shPRMT5-KD (n=3) tumors was performed using Image J. Data are presented as means ±sem. FIG. 7D depicts immune phenotyping performed using flow cytometry and indicated cell surface markers in YUMMER1.7 tumors collected at day 12. FIGS. 7E-H depict B16 cells stably expressing control (Scr) or PRMT5 shRNA (shPRMT5) were grafted into C57BL/6 mice (n=8) administered control (IgG) or neutralizing antibodies against either NK1.1 (200 μg/mouse FIGS. 7E and 7F) or CD8+(200 μg/mouse FIGS. 7G and 711) every three days starting one day prior to tumor inoculation. Shown are tumor volumes (FIGS. 7E and 7G) and percent survival (FIGS. 7F and 711). Data are presented as means ±s.d., unless specified. *, **, *** and **** represent p<0.05, p<0.01, p<0.001 and p<0.0001, respectively.

FIGS. 8A-H depict PRMT5 methylation of the cGAS complex component IFI16/IFI204. FIG. 8A depicts melanoma patient specimens expressing comparable PRMT5 levels were grouped based on low or high levels of PRMT5 adaptor proteins (namely, SHARPIN, WDR77, RIOK1, COPRS, CLNS1A, and MEN1). Differentially-expressed genes (DEGs) were analyzed using GSEA. FIG. 8B depicts a heat map depicting normalized enrichment score (NES) and q value of false discovery rate (FDR-q) for PRMT5 adaptor proteins in immune-associated hallmark gene sets. FIG. 8C depicts immunoprecipitation (IP) followed by immunoblotting (IB) of WM115 cell lysates (1.2 mg) with indicated antibodies. FIG. 8D depicts B16 cells were treated with vehicle (DMSO) or PRMT5 inhibitor (PRMT5i; EPZ015666, 10 μM) for 48. IP followed by IB of B16 cells lysates (1.5 mg) was performed with the indicated antibodies. SYM10 indicates anti-symmetric dimethyl arginine antibody (Millipore). FIGS. 8E and 8F depict A375 (FIG. 8E) or B16 (FIG. 8F) cells treated with vehicle or a PRMT5 inhibitor (EPZ015666) as above before lysates [A375 (1.0 mg), B16 (2.5 mg)] were prepared and subjected to IP followed by immunoblotting with indicated antibodies. FIG. 8G depicts B16 cells stably expressing indicated constructs were treated 24 h with DMSO or PRMT5i before lysates were IP'ed with V5 antibody and immunoblotted with indicated antibodies. WT: IFI204 WT; Mt1, Mt2 and Mt1/2: IFI204 mutants R12A, R538A or RR12/538AA, respectively; EV: empty vector. FIG. 811 depicts in vitro methylation assay of WT or mutant IFI204 proteins (200 ng) purified from HEK293T cell lysates, with or without recombinant active PRMT5 plus WDR77 (500 ng) proteins. Proteins were visualized using PonceauS and InstantBlue staining (lower panels) and subjected to autoradiography (upper panel). Histone 4 served as a positive control.

FIGS. 9A-B depict expression of PRMT5 adaptors in low PRMT5 melanoma specimens. FIG. 9A depicts low PRMT5 specimen (red box on left) were selected (100/368 as shown in right panel) for GSEA analysis. FIG. 9B depicts relative [Low (L) vs. High (H) expression of indicated adaptors in the low PRMT5 tumor cohort selected as in FIG. 9A. Additional GSEA analysis is shown in FIG. 8B.

FIGS. 10A-F depict methylation of SHARPIN-interacting proteins IFI16 and IFI204. FIGS. 10A and lOB depict IFI16 (FIG. 10A) or IFI204 (Fig. lOB) interaction with Flag-SHARPIN in HEK293T cells transfected with indicated constructs followed by Flag IP of cell lysates and immunoblot with indicated antibodies. Red arrow heads indicate position of respective interacting proteins. FIG. 10C depicts immunoblot analysis of indicated proteins in A375 cells following IFI16 or IgG IP from PRMT5i (EPZ015666, 10 μN)-treated cells. Control input depicts Arg methylation following PRMT5i treatment. FIG. 10D depicts IFI16 methylation levels in WM115 cells treated with EPZ015666 (10 μM) relative to non-treated cells. Arg methylation was quantified relative to amounts of IP'd IFI16 protein. Control input depicts Arg methylation following inhibitor treatment. FIG. 10E depicts schematic of IFI16 and IFI204 domain structure. Arrowheads indicate putative PRMT5 Arg methylation residues. FIG. 10F depicts HEK293T cells were transfected with V5-tagged-IFI204 and 24h later treated with vehicle (DMSO, IFI204) or PRMT5i (EPZ015666 10 IFI204*). Cell lysates prepared 24h later (left panel) were used for IP of V5-tagged-IFI204. Immunopurified IFI204 or IFI204* (500 ng) was analyzed using InstantBlue staining (left panel) and to in vitro methylation using recombinant PRMT5 /WDR77 (550 ng) monitored by autoradiography (right panel). H4 (1 μg) was used as a positive control.

FIGS. 11A-K depict PRMT5 methylation of IFI204 determined degree of cGAS/STING pathway activation. FIGS. 11A, 11B, and 11C depict B16 cells were transduced with either scramble (Scr) or Prmt5-specific shRNAs (shPRMT5-1, shPRMT5-2) (FIG. 11A), treated for 24 hr with PRMT5i (FIG. 11B), or subjected to ectopic expression of control (pLX304 and pLenti) or PRMT5+WDR77 (pLX304-WDR77/pLenti-PRMT5) (FIG. 11C). Following respective treatments, cells were stimulated with dsDNA (transfected V70mer; 500 ng/ml). Six hours later cell lysates were prepared and assayed using qPCR for expression of indicated transcripts. FIGS. 11D, 11E, and 11F depict analysis of cGAS/STING complex components by western blot analysis (FIG. 11D), semi-native-PAGE (FIG. 11E), or BlueNative-PAGE (FIG. 11F) of proteins prepared from B16 cells subjected to PRMT5 KD using corresponding shRNA (as in FIG. 11A) followed by stimulation with dsDNA (V70mer; 1.5 μg/ml) for indicated times. Lower panels show Ponceau S staining (lower panels in FIGS. 11E, 11F) “d” and “m” (FIG. 11E) represent “dimer” and “monomer” forms of STING. FIGS. 11G and 11H depict analysis of cGAS/STING complex components with indicated antibodies using western blot analysis of lysates prepared from B16 cells either treated with PRMT5i (as in FIG. 11B) or stably expressing PRMT5+WDR77 (as in FIG. 11C) following stimulation with dsDNA (transfected V70mer; 1.5 μg/ml) for indicated times. FIG. 11I depicts B16 cells stably expressing pLX304 (EV), IFI204WT (WT), the IFI204R12A mutant (Mt1) or the IFI204R538A mutant (Mt2) were transfected with V70mer (500 ng/ml) for 6 hr and then assessed for expression of indicated transcripts by qPCR. FIG. 11I depicts B16 cells stably expressing IFI204 plasmids (as in FIG. 11I) were transfected with V70mer (1.5 μg/ml) for indicated times followed by analysis of cell lysates by semi-native-PAGE blotting with indicated antibodies and Ponceau S staining. STING dimer (d) and monomer (m) forms are noted. FIG. 11K depicts B16 cells transduced with Scr or Prmt5-specific shRNAs were transfected with scrambled control (siCont) or Sting-specific (siSting) siRNAs for 48 hr. Cells were then stimulated 6 h with dsDNA (transfected V70mer; 500 ng/ml) before lysates were prepared for qPCR analysis of indicated transcripts. Western blot inset depicts level of STING expression. Data are presented as means ±s.d. *, **, *** and **** represent p<0.05, p<0.01, p<0.001 and p<0.0001, respectively.

FIGS. 12A-K depict PRMT5 -SHARPIN attenuation and IFI204 augmentation of STING activity. FIG. 12A depicts YUMMER1.7 cells were harvested 6 h following their transfection with dsDNA (V70mer; 500 ng/ml), and relative levels of indicated transcripts assessed by qPCR. FIGS. 12B and 12C depict B16F10 cells expressing empty vector (EV) or SHARPIN were transfected with dsDNA (V70mer; 1.5 μg/ml) for indicated times or dsDNA (V70mer; 500 ng/ml) for 6hr (FIG. 12C) and cell lysates were immunoblotted with indicated antibodies (FIG. 12B) or subjected to qPCR analysis of indicated transcripts (FIG. 12C). FIG. 12D depicts YUMMER1.7 cells expressing EV or PRMT5/WDR77 were transfected with dsDNA (V70mer; 1.5 μg/ml) for indicated times and then cell lysates analyzed by immunoblotting with indicated antibodies. FIGS. 12E and 12F depict B16F10 cells stably expressing empty vector (EV) or WT IFI204 (#1 and #2 represent independent reactions) were transfected with dsDNA [V70mer; 1.5 μg/m1 (FIG. 12E) or 500 ng/ml (FIG. 12F)]. Cell lysates prepared 6 h later were subjected to immunoblotting with indicated antibodies (FIG. 12E) or qPCR analysis of indicated transcripts (FIG. 12F). FIGS. 12G and 1211 depict B16F10 cells stably expressing EV or indicated IFI204 plasmids were transfected with dsDNA (V70mer; 1.5 μg/ml) for indicated times. Cell lysates were then analyzed by BlueNative-(upper blots) or SDS-(lower blots)-PAGE with indicated antibodies. B16F10 cells transduced with Scr or PRMT5 shRNAs were transfected with scrambled control (siCont) or Sting (siSting) siRNAs for 48 hr. Cells were then transfected with dsDNA (V70mer; 500 ng/ml), and 6 h later harvested for qPCR analysis of Prmt5 and Sting transcripts. FIG. 12I depicts expression of cGAS and STING/TMEM173 was assessed in B16F10 cells stably expressing indicated plasmids or shRNA. FIG. 12J depicts B16F10 cells stably transduced with Scr or shPRMT5 lentivirus were transfected with LMW or HMW poly(I:C) (250 ng/ml) or V70mer (500 ng/ml) for 6 hr. Expression of indicated transcripts was assessed by qPCR. FIG. 12K depicts B16F10 cells stably expressing EV or indicated IFI204 plasmids were transfected with poly(I:C) or V70mer and expression of indicated transcripts was assessed.

FIGS. 13A-H depict inverse correlation of NLRCS expression with PRMT5. FIG. 13A depicts genes differentially expressed relative to PRMT5 levels (2-fold difference with p<0.05, n=155) were identified in analysis of the TCGA dataset (upper left) and compared with PRMT5 -co-regulated genes (r>5 or r<-5 in Pearson's correlation, n=135) identified in analysis of the CCLE dataset (upper right). Among the nine genes that were found correlate with low PRMT5 expression in both CCLE and TCGA analyses was NLRCS (lower table). Ingenuity Pathway Analysis (IPA) identified NLRCS as associated with antigen presentation pathways (lower diagram), which were also associated with PRMT5 expression. FIG. 13B depicts Pearson's correlation analysis of PRMT5 and antigen presentation genes in the TCGA dataset. Genes above light blue line: p<0.05. FIG. 13C depict NLRCS expression, as analyzed in the GSE80182 dataset from A549 cells following depletion of MEP50 (WDR77) or PRMT5 . FIG. 13D depict Nlrc5 transcript levels in YUMMER1.7 cells stably expressing EV or PRMT5 +WDR77. FIG. 13E depicts B16F10 cells were stimulated 24 hr with interferon gamma (at indicated concentrations) and PSMB9 expression was monitored. FIGS. 13F-G depict B16F10 cells stably expressing NLRCS were stimulated 24 hr with interferon gamma and then cell lysates were analyzed by immunoblotting with indicated antibodies (FIG. 13F). Surface MHCI (H-2Kb) expression, was assessed by flow cytometry (FIG. 13G). FIG. 13H depicts surface expression of interferon gamma receptor beta (IFNGR2), was assessed using flow cytometry of B16F10 cells stably transduced with scrambled (Scr) or shPRMT5.

FIGS. 14A-H depict negative regulation of NLRC5 by PRMT5 to modulate MHCI antigen presentation. FIG. 14A depicts correlation of PRMT5 expression with that of genes implicated in antigen presentation in melanoma lines (CCLE, cancer cell line encyclopedia datasets, n=58) was evaluated using Pearson's correlation coefficient (plotted on X-axis) and −log (p value) (plotted on the Y-axis). Blue horizontal line indicates cutoff level for p<0.05. FIG. 14B depicts Pearson's correlation of PRMT5 and NLRC5 mRNA expression in melanoma cell lines (CCLE, n=58). FIG. 14C depicts Pearson's correlation of PRMT5 and NLRC5 mRNA expression in melanoma patient specimens (TCGA, n=368). FIGS. 14D-F depict qPCR analysis of genes implicated in antigen presentation was performed in B16 cells either transduced with Scr or Prmt5-specific shRNAs (shPRMT5-1, shPRMT5-2) (FIG. 14D), treated with PRMT5i (MTA, 100 μM for 24 hr) (FIG. 14E), or stably expressing EV or PRMT5+WDR77 (FIG. 14F). FIG. 14G depicts immunoblotting of lysates of B16 cells transduced with scrambled (Scr) or shPRMT5 and treated 24 hr with indicated concentrations (ng /ml) of interferon gamma (IFN gamma) using antibodies to indicated proteins. FIG. 14H depicts cell surface MHCI expression (H-2Kb) in B16 cells subjected to indicated treatments, as assessed by flow cytometry (left). Quantification of mean fluorescence intensity (MFI) (right). Data are presented as means ±s.d. *, **, *** and **** represent p<0.05, p<0.01, p<0.001 and p<0.0001, respectively.

FIGS. 15A-E depict inhibition of melanoma growth by co-expression of mutant IFI16/IFI204 and NLRC5. FIG. 15A depicts B16 cells were transduced with EV or expression vectors harboring IFI204Mt1 and/or NLRC5 and then analyzed by Western blotting for indicated proteins (FIG. 15A). FIG. 15B depicts tumor growth was assessed in mice (n=8) grafted with B16 cells established in (FIG. 15A). FIG. 15C depicts growth of B16 cells in culture (established as shown in FIG. 15A) was monitored using ATPlite assay. Data are presented as means ±s.d. Statistical significance of changes in tumor growth and cell growth were assessed using two-way ANOVA with Tukey's correction and one-way ANOVA with Dunnett's test. FIGS. 15D-E depicts in left panels classification of specimens based on low or high levels of IFI16 (FIG. 15D) or NLRC5 (FIG. 15E) expression (based on TCGA, metastatic population of melanoma, n=368). Right panels show overall survival of melanoma patients based on relative expression of IFI16 (FIG. 15D) or NLRC5 (FIG. 15E).

FIGS. 16A-D depict expression of methylation mutant IFI16 and NLRC5 in B16F10 tumors increasing T cell infiltration correlated with enriched immune-associated gene sets. FIG. 16A depicts B16F10 melanoma tumors stably transduced with empty vectors (EV+EV) or IFI204Mt1+NLRC5 plasmids were inoculated in C57BL/6 mice for 8 days before tumors were harvested and subjected to immunohistochemistry for CD8+ and CD4+ immune cell infiltration (upper panel). The “Ratio of area” was calculated by dividing the area of CD4+ or CD8+ signals by that of DAPI+ signals using Image J. Data are represented as mean ±sem (lower panel). FIG. 16B depicts expression of indicated PRMTs' transcripts was assessed in B16F10 melanoma cells stably transduced with empty vectors (EV+EV) or IFI204Mt1+NLRC5 plasmids by qPCR. FIGS. 16C-D depict differentially expressed genes in tumors harboring low or high expression of IFI16 (FIG. 16C) or NLRC5 (FIG. 16D), as analyzed using GSEA.

FIGS. 17A-G depict synergistic effect of PRMT5 inhibition with anti-PD-1 immune check-point therapy. FIG. 17A depicts a schematic for PRMT5 control of IFN/chemokine expression and antigen presentation pathways. FIGS. 17B and 17C depict expression of transcripts encoding indicated IFN/chemokines (FIG. 17B) and immune checkpoint components (FIG. 17C) based on qPCR of tumors transduced with control (Scr-KD) or PRMT5 KD (shPRMT5), 17 days after tumor cell inoculation. FIG. 17D depicts B16 cells transduced with scrambled (Scr) or shPRMT5 were grafted into C57BL/6 mice (n=8) subsequently treated with control IgG or anti-PD-1 antibody (200 μg/mouse at days, 8, 11, 14, 17 and 20). Tumor volume (upper) and percent survival (lower) were assessed at indicated time points. FIG. 17E depicts B16 cells were grafted into syngeneic C57BL/6 mice (n=6-8) subsequently treated with PRMT5i (GSK3326595, 40 mg/kg from day 10) and/or anti-PD-1 antibody (200 μg/mouse at days 11, 14 and 17). Tumor volume (upper) and percent survival (lower) were assessed at indicated time points. FIG. 17F depicts YUMM1.7 cells were grafted into syngeneic C57BL/6 mice (n=7-8) subsequently treated with PRMT5i (from day 7) and/or anti-PD-1 antibody (at days 8, 11, 14 and 17). Tumor volume (upper) and percent survival (lower) were monitored at indicated time points. FIG. 17G depicts YUMM1.7 cells were grafted into syngeneic C57BL/6 mice (n=7) subsequently administered anti-CD8+ antibody (200 μg/mouse) every three days, starting one day prior to tumor inoculation. As indicated, mice were also administered PRMT5i (from day 8) and/or anti-PD-1 antibody at days 9, 12, 15 and 18. Tumor volume (upper) and percent survival (lower) were monitored at indicated time points. For statistical analyses, tumor response was calculated based on tumor volume and percent survival, using Fisher's exact test and a log-rank test, respectively. *, **, *** and **** represent p<0.05, p<0.01, p<0.001 and p<0.0001, respectively.

FIGS. 18A-J depict genetic or pharmacological PRMT5 inhibition augment effect of therapeutic effect of anti-PD-1 blockade. FIG. 18A depicts B16F10 cells transduced with scrambled (Scr) or shPRMT5 were grafted into C57BL/6 mice (n=8) subsequently treated with control IgG or anti-PD-1 antibody (200 μg/mouse at days, 8, 11, 14, 17 and 20). Tumor volume were assessed at indicated time points. Red horizontal line indicates the volume (2,000 mm³). FIG. 18B depicts B16F10 cells were grafted into syngeneic C57BL/6 mice, which were then treated with indicated doses of PRMT5i (GSK3326595 for 1 week starting day 10). Tumors were collected and monitored for the inhibition of PRMT5 activity using Arg-methyl-specific antibody. FIG. 18C depicts B16F10 cells were grafted into syngeneic mice (n=6-7), which were then treated with PRMT5i (GSK3326595, 40 mg/kg, QD from day 6) and/or anti-PD1 antibody (administered on days 8, 11, 14, and 17). Tumor growth was assessed by tumor volume. Statistical significance was analyzed with Fisher's exact test. FIG. 18D depicts percent survival was calculated using Kaplan-Meier plot and significance was determined by log-rank test. FIG. 18E depicts B16F10 cells were grafted to C57BL/6 mice and treated with vehicle (n=4) or PRMT5i (GSK3326595, 40 mg/kg, QD; n=4) starting 8 days following tumor inoculation. Immune phenotyping performed using flow cytometry with the indicated cell surface markers and intracellular cytokines on vehicle or PRMT5i-treated B16 tumors collected at day 15. FIGS. 18F-18G depicts weight and PRMT5 activity (Arg-methyl staining) were assessed in tumors collected in FIG. 18E. FIG. 1811 depicts C57BL/6 mice (n=7) were treated with control IgG or anti-CD8+ antibody (200 μg/mouse every three days starting one day prior to treatment until the end of the experiment). Mouse peripheral blood was collected at day 8 and assessed for efficient CD8+ cell depletion by flow cytometry. FIGS. 18I-18J depicts B16 cells were grafted into syngeneic C57BL/6 mice (n =5-7) that were subsequently treated with PRMT5i (GSK3326595, 40 mg/kg from day 7) and/or anti-CTLA-4 antibody (100 μg/mouse at days 8, 11, 14, 17 and 21). Tumor volume (FIG. 18I) and percent survival (FIG. 18J) were assessed at indicated time points.

FIGS. 19A-D depict CRISPR gene-editing of Prmt5. FIG. 19A shows B16F10 cells expressing Cas9 transfected with indicated gRNAs specific to Prmt5 gene. gRNA-mediated creation of indels was quantified by determining the percent cleavage of genomic DNA from pool of B16 cells collected 2 days after gRNA transfection. FIG. 19 B depicts PRMT5 expression and activity assessed in B16F10 cells collected 4 days after gRNA transfection by immunoblotting with indicated antibodies. FIG. 19C shows B16F10 cells transfected with gRNA #779 were subjected to serial dilution to generate single cell clones. PRMT5 expression and activity were assessed using indicated antibodies. FIG. 19D shows presence of gRNA-induced indels at the Prmt5 target locus was assessed by genomic DNA cleavage in B16F10 subclones #6 and #12.

### DETAILED DESCRIPTION

Certain specific details of this description are set forth in order to provide a thorough understanding of various embodiments. However, one skilled in the art will understand that the present disclosure may be practiced without these details. In other instances, well-known structures and/or methods have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All references cited herein are incorporated by reference in their entirety as though fully set forth. Singleton et al., Dictionary of Microbiology and Molecular Biology 3rd ed., J. Wiley & Sons (New York, NY 2001); March, Advanced Organic Chemistry Reactions, Mechanisms and Structure 5th ed., J. Wiley & Sons (New York, NY 2001); and Sambrook and Russel, Molecular Cloning: A Laboratory Manual 3rd ed., Cold Spring Harbor Laboratory Press (Cold Spring Harbor, NY 2001), provide one skilled in the art with a general guide to many of the terms used in the present application.

### Definitions

When indicating the number of substituents, the term “one or more” refers to the range from one substituent to the highest possible number of substitution, e.g. replacement of one hydrogen up to replacement of all hydrogens by substituents.

The term “optional” or “optionally” denotes that a subsequently described event or circumstance can but need not occur, and that the description includes instances where the event or circumstance occurs and instances in which it does not.

The term “nucleic acid” as used herein generally refers to one or more nucleobases, nucleosides, or nucleotides, and the term includes polynucleobases, polynucleosides, and polynucleotides.

The term “polynucleotide”, as used herein generally refers to a molecule comprising two or more linked nucleic acid subunits, e.g., nucleotides, and can be used interchangeably with “oligonucleotide”. For example, a polynucleotide may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (P03) groups. A nucleotide can include a nucleobase, a five-carbon sugar (either ribose or deoxyribose), and one or more phosphate groups. Ribonucleotides include nucleotides in which the sugar is ribose. Deoxyribonucleotides include nucleotides in which the sugar is deoxyribose. A nucleotide can be a nucleoside monophosphate, nucleoside diphosphate, nucleoside triphosphate or a nucleoside polyphosphate. For example, a nucleotide can be a deoxyribonucleoside polyphosphate, such as a deoxyribonucleoside triphosphate (dNTP), Exemplary dNTPs include deoxyadenosine triphosphate (dATP), deoxycytidine triphosphate (dCTP), deoxyguanosine triphosphate (dGTP), uridine triphosphate (dUTP) and deoxythymidine triphosphate (dTTP). dNTPs can also include detectable tags, such as luminescent tags or markers (e.g., fluorophores). For example, a nucleotide can be a purine (e.g., A or G, or variant thereof) or a pyrimidine (e.g., C, T or U, or variant thereof). In some examples, a polynucleotide is deoxyribonucleic acid (DNA), ribonucleic acid (RNA), or derivatives or variants thereof. Exemplary polynucleotides include, but are not limited to, short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), and heteronuclear RNA (hnRNA), and encompasses both the nucleotide sequence and any structural embodiments thereof, such as single-stranded, double-stranded, triple-stranded, helical, hairpin, stem loop, bulge, etc. In some cases, a polynucleotide is circular. A polynucleotide can have various lengths. For example, a polynucleotide can have a length of at least about 7 bases, 8 bases, 9 bases, 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. For example, polynucleotide sequences may comprise isolated and purified DNA/RNA molecules, synthetic DNA/RNA molecules, and/or synthetic DNA/RNA analogs.

Polynucleotides may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino- 3- N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine -modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NETS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev DA, Lavergne T, Welte W, Diederichs K, Dwyer TJ, Ordoukhanian P, Romesberg FE, Marx A. Nat. Chem. Biol. 2012 July;8(7):612-4, which is herein incorporated by reference for all purposes.

As used herein, the terms “polypeptide”, “protein” and “peptide” are used interchangeably and refer to a polymer of amino acid residues linked via peptide bonds and which may be composed of two or more polypeptide chains. The terms “polypeptide”, “protein” and “peptide” refer to a polymer of at least two amino acid monomers joined together through amide bonds. An amino acid may be the L-optical isomer or the D-optical isomer. More specifically, the terms “polypeptide”, “protein” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant.

As used herein, the term “biological sample” means any biological material from which polynucleotides, polypeptides, biomarkers, and/or metabolites can be prepared and examined. Non-limiting examples encompasses whole blood, plasma, saliva, cheek swab, fecal specimen, urine specimen, cell mass, or any other bodily fluid or tissue.

The terms “administer,” “administering”, “administration,” and the like, as used herein, refer to the methods that may be used to enable delivery of compounds or compositions to the desired site of biological action. These methods include, but are not limited to oral routes (p.o.), intraduodenal routes (i.d.), parenteral injection (including intravenous (i.v.), subcutaneous (s.c.), intraperitoneal (i.p.), intramuscular (i.m.), intravascular or infusion (inf.)), topical (top.) and rectal (p.r.) administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated; for example a reduction and/or alleviation of one or more signs, symptoms, or causes of a disease, or any other desired alteration of a biological system. For example, an “effective amount” for therapeutic uses can be an amount of an agent that provides a clinically significant decrease in one or more disease symptoms. An appropriate “effective” amount may be determined using techniques, such as a dose escalation study, in individual cases.

The terms “enhance” or “enhancing,” as used herein, means to increase or prolong either in amount, potency or duration a desired effect. For example, in regard to enhancing expression of a gene, the term “enhancing” can refer to the ability to increase the level of mRNA or protein encoded by the gene.

The terms “inhibitor” or “inhibitory agent” as used herein encompass compositions, agents, and compounds that inhibit expression or activity of a gene or protein. “Inhibit,” “inhibiting,” and “inhibition” and like terms include decreasing an activity, response, condition, disease, or other biological parameter. This can include but is not limited to the complete ablation of the expression, activity, response, condition, or disease. This may include, for example, a 10% reduction in the expression, activity, response, condition, or disease as compared to the native or control level. Thus, the reduction can be a 10, 20, 30, 40, 50, 60, 70, 80, 90, 100%, or any amount of reduction in between as compared to native or control levels.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a non-human animal is a mouse.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “preventing” or “prevention” of a disease state denotes causing the clinical symptoms of the disease state not to develop in a subject that can be exposed to or predisposed to the disease state, but does not yet experience or display symptoms of the disease state.

The terms “pharmaceutical composition” and “pharmaceutical formulation” (or “formulation”) are used interchangeably and denote a mixture or solution comprising a therapeutically effective amount of an active pharmaceutical ingredient together with one or more pharmaceutically acceptable excipients to be administered to a subject, e.g., a human in need thereof

The term “pharmaceutical combination” as used herein, means a product that results from mixing or combining more than one active ingredient and includes both fixed and non-fixed combinations of the active ingredients. The term “fixed combination” means that the active ingredients, e.g., a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., administration of three or more active ingredients.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, e.g., the material may be administered to an individual without causing undesirable biological effects or interacting in a deleterious manner with any of the components of the composition in which it is contained.

The terms “pharmaceutically acceptable excipient”, “pharmaceutically acceptable carrier”, “pharmaceutically acceptable vehicle” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products

The term “pharmaceutically acceptable salts” denotes salts which are not biologically or otherwise undesirable. Pharmaceutically acceptable salts include both acid and base addition salts. A “pharmaceutically acceptable salt” can refer to a formulation of a compound or agent that does not cause significant irritation to an organism to which it is administered and/or does not abrogate the biological activity and properties of the compound or agent.

Methods for detection and/or measurement of polypeptides in biological material are well known in the art and include, but are not limited to, Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Methods for detection and/or measurement of RNA in biological material are well known in the art and include, but are not limited to, Northern-blotting, RNA protection assay, RT PCR. Suitable methods are described in Molecular Cloning: A Laboratory Manual (Fourth Edition) By Michael R. Green, Joseph Sambrook, Peter MacCallum 2012, 2,028 pp, ISBN 978-1-936113-42-2.

A ribonucleoprotein (RNP) refers to a nucleoprotein that contains RNA. A RNP can be a complex of a ribonucleic acid and an RNA-binding protein. Such a combination can also be referred to as a protein-RNA complex. These complexes can function in a number of biological functions that include, but are not limited to, DNA replication, gene expression, metabolism of RNA, and pre-mRNA splicing. Examples of RNPs include the ribosome, the enzyme telomerase, vault ribonucleoproteins, RNase P, heterogeneous nuclear RNPs (hnRNPs) and small nuclear RNPs (snRNPs).

As used herein, the term “biomarker” or “marker” are used interchangeably to refer to any biochemical marker, serological marker, genetic marker, or other clinical or echographic characteristic that can be used to classify a sample from a patient as being associated with a tumor condition, including pancreatic cancer and melanoma. The recitation of specific examples of markers associated with tumor conditions is not intended to exclude other markers as known in the art and suitable for use in the present disclosure.

As used herein, the term “antibody” includes but is not limited to a population of immunoglobulin molecules, which can be polyclonal or monoclonal and of any class and isotype, or a fragment of an immunoglobulin molecule. There are five major classes of immunoglobulins: IgA, IgD, IgE, IgG, and IgM, and several of these may be further divided into subclasses (isotypes), e.g., IgG1, IgG2, IgG3, IgG4, IgA1 (human), IgA2 (human), IgAa (canine), IgAb (canine), IgAc (canine), and IgAd (canine). Such fragment generally comprises the portion of the antibody molecule that specifically binds an antigen. For example, a fragment of an immunoglobulin molecule known in the art as Fab, Fab′ or F(ab′)2 is included within the meaning of the term antibody.

As used herein, the term “neutralizing antibody” includes antibodies which are capable of specifically binding to an epitope on a protein and neutralizing the protein. Neutralizing antibodies also include antibodies which are capable of binding to an epitope on a protein and rendering the protein inactive. Neutralizing antibodies also include antibodies which are capable of inhibiting binding of a protein to its receptor. For example, the neutralizing antibodies provided herein may be capable of binding to and neutralizing a PRMT5 protein. In some embodiments, the neutralizing antibodies include recombinant and chimeric antibodies. In some embodiments, the neutralizing antibodies include human antibodies. In some embodiments, the neutralizing antibodies include a human variable region. In some embodiments, the neutralizing antibodies include a human light chain constant region. In some embodiments, the neutralizing antibodies include a human heavy chain constant region.

As used herein, the term “endogenous antibodies” refers to antibodies made by or originating from a subject, which can be isolated from the patient's blood or tissue. Typically, endogenous antibodies are generated in response to a foreign antigen, for example in response to a bacterial antigen, as part of the body's natural defense against infection. In certain cases, however, the patient may generate endogenous antibodies against the body's own proteins, such endogenous antibodies being referred to herein as “autoantibodies”.

The term “label,” as used herein, refers to a detectable compound, composition, or solid support, which can be conjugated directly or indirectly (e.g., via covalent or non-covalent means, alone or encapsulated) to a monoclonal antibody or a protein. The label may be detectable by itself (e.g., radioisotope labels, chemiluminescent dye, electrochemical labels, metal chelates, latex particles, or fluorescent labels) or, in the case of an enzymatic label, may catalyze chemical alteration of a substrate compound or composition which is detectable (e.g., enzymes such as horseradish peroxidase, alkaline phosphatase, and the like). The label employed in the current discolosure could be, but is not limited to alkaline phosphatase; glucose-6-phosphate dehydrogenase (“G6PDH”); horseradish peroxidase (HRP); chemiluminescers such as isoluminol, fluorescers such as fluorescein and rhodamine compounds; ribozymes; and dyes. The label may also be a specific binding molecule which itself may be detectable (e.g., biotin, avidin, streptavidin, digioxigenin, maltose, oligohistidine, e.g., hex-histidine, 2, 4-dinitrobenzene, phenylarsenate, ssDNA, dsDNA, and the like). The utilization of a label produces a signal that may be detected by means such as detection of electromagnetic radiation or direct visualization, and that can optionally be measured.

A monoclonal antibody can be linked to a label using methods well known to those skilled in the art, e.g., Immunochemical Protocols; Methods in Molecular Biology, Vol. 295, edited by R. Bums (2005). For example, a detectable monoclonal antibody conjugate may be used in any known diagnostic test format like ELISA or a competitive assay format to generate a signal that is related to the presence or amount of an IBD-associated antibody in a test sample.

“Substantial binding” or “substantially binding” refer to an amount of specific binding or recognizing between molecules in an assay mixture under particular assay conditions. In its broadest aspect, substantial binding relates to the difference between a first molecule's incapability of binding or recognizing a second molecule, and the first molecules capability of binding or recognizing a third molecule, such that the difference is sufficient to allow a meaningful assay to be conducted to distinguish specific binding under a particular set of assay conditions, which includes the relative concentrations of the molecules, and the time and temperature of an incubation. In another aspect, one molecule is substantially incapable of binding or recognizing another molecule in a cross-reactivity sense where the first molecule exhibits a reactivity for a second molecule that is less than 25%, e.g. less than 10%, e.g., less than 5% of the reactivity exhibited toward a third molecule under a particular set of assay conditions, which includes the relative concentration and incubation of the molecules. Specific binding can be tested using a number of widely known methods, e.g, an immunohistochemical assay, an enzyme-linked immunosorbent assay (ELISA), a radioimmunoassay (RIA), or a western blot assay.

As used herein, the term “substantially the same amino acid sequence” includes an amino acid sequence that is similar, but not identical to, the naturally-occurring amino acid sequence. For example, an amino acid sequence, e.g., polypeptide, that has substantially the same amino acid sequence as a IFI204 protein can have one or more modifications such as amino acid additions, deletions, or substitutions relative to the amino acid sequence of the naturally-occurring flagellin protein, provided that the modified polypeptide retains substantially at least one biological activity of flagellin such as immunoreactivity. The “percentage similarity” between two sequences is a function of the number of positions that contain matching residues or conservative residues shared by the two sequences divided by the number of compared positions times 100. In this regard, conservative residues in a sequence is a residue that is physically or functionally similar to the corresponding reference residue, e.g., that has a similar size, shape, electric charge, chemical properties, including the ability to form covalent or hydrogen bonds, or the like.

The term “heterologous” refers to any two or more nucleic acid or polypeptide sequences that are not normally found in the same relationship to each other in nature. For instance, a heterologous nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

As used herein, the term “fragment” includes a peptide, polypeptide or protein segment of amino acids of the full-length protein, provided that the fragment retains reactivity with at least one antibody in sera of disease patients.

An “epitope” is the antigenic determinant on a polypeptide that is recognized for binding by a paratope on antibodies specific to the polypeptide, for example, an PRMT5 antibody or a PRMT5-associated antibody.

Provided herein are compositions, methods, and compounds for diagnosis, treatment, determining, monitoring, and selecting treatment of cancer, and preferably of melanoma. In particular aspects, provided herein are compositions and methods for regulation of control of antitumor immunity. Compositions and methods provided herein, for example, pharmaceutical compositions and treatments may be capable of regulating the modification, expression, and/or activity of protein factors and complexes involved in tumor intrinsic immune response, including the cCAS/STIING complex. In some aspects, the pharmaceutical compositions and treatment provided herein further encompass use of immune regulators including checkpoint inhibitors. For example, a PRMT5 inhibitor may be co-administered with an immune checkpoint therapy. The compositions and methods described herein provides approaches for regulating and enhancing antigen processing and presentation, and antitumor immunity.

### Compositions Enhancing Antitumor Immune Response

Provided herein are compositions for use in treatment of cancer. Such compositions may include isolated or purified proteins, polypeptides or any fragment thereof, polynucleotides or any fragment thereof, antibodies, vectors, protein complexes, protein-polynucleotide complexes, and/or small molecules. The compositions provided herein may treat, alleviate the symptoms of, delay, or reduce the likelihood of tumors or cancers. Non-limiting examples of tumors that may be treated with methods and compositions disclosed herein include acute lymphoblastic leukemia (adult), acute lymphoblastic leukemia (childhood), acute myeloid leukemia (adult), acute myeloid leukemia (childhood), adrenocortical carcinoma , adrenocortical carcinoma (childhood), AIDS-related cancers, AIDS-related lymphoma, anal cancer, astrocytoma (childhood cerebellar), astrocytoma (childhood cerebral), basal cell carcinoma, bile duct cancer (extrahepatic), bladder cancer, bladder cancer (childhood), bone cancer (osteosarcoma/malignant fibrous histiocytoma), brain stem glioma (childhood), brain tumor (adult), brain tumor-brain stem glioma (childhood), brain tumor-cerebellar astrocytoma (childhood), brain tumor-cerebral astrocytoma/malignant glioma (childhood), brain tumor -ependymoma (childhood), brain tumor-medulloblastoma (childhood), brain tumor -supratentorial primitive neuroectodermal tumors (childhood), brain tumor-visual pathway and hypothalamic glioma (childhood), breast cancer (female, male, childhood), bronchial adenomas/carcinoids (childhood), Burkitt's lymphoma, carcinoid tumor (childhood), carcinoid tumor (gastrointestinal), carcinoma of unknown primary site (adult and childhood), central nervous system lymphoma (primary), cerebellar astrocytoma (childhood), cerebral astrocytoma/malignant glioma (childhood), cervical cancer, chronic lymphocytic leukemia, chronic myelogenous leukemia, chronic myeloproliferative disorders, colon cancer, colorectal cancer (childhood), cutaneous t-cell lymphoma, endometrial cancer, ependymoma (childhood), esophageal cancer, esophageal cancer (childhood), Ewing's family of tumors, extracranial germ cell tumor (childhood), extragonadal germ cell tumor, extrahepatic bile duct cancer, eye cancer (intraocular melanoma and retinoblastoma), gallbladder cancer, gastric (stomach) cancer, gastric (stomach) cancer (childhood), gastrointestinal carcinoid tumor, gastrointestinal stromal tumor (gist), germ cell tumor (extracranial (childhood), extragonadal, ovarian), gestational trophoblastic tumor, glioma (adult), glioma (childhood: brain stem, cerebral astrocytoma, visual pathway and hypothalamic), hairy cell leukemia, head and neck cancer, hepatocellular (liver) cancer (adult primary and childhood primary), Hodgkin's lymphoma (adult and childhood), Hodgkin's lymphoma during pregnancy, hypopharyngeal cancer, hypothalamic and visual pathway glioma (childhood), intraocular melanoma, islet cell carcinoma (endocrine pancreas), Kaposi's sarcoma, kidney (renal cell) cancer, kidney cancer (childhood), laryngeal cancer, laryngeal cancer (childhood), leukemia -acute lymphoblastic (adult and childhood), leukemia, acute myeloid (adult and childhood), leukemia-chronic lymphocytic, leukemia-chronic myelogenous, leukemia -hairy cell, lip and oral cavity cancer, liver cancer (adult primary and childhood primary), lung cancer -non-small cell, lung cancer-small cell, lymphoma-AIDS-related, lymphoma -Burkitt's, lymphoma -cutaneous t-cell, lymphoma -Hodgkin's (adult, childhood and during pregnancy), lymphoma -non-Hodgkin's (adult, childhood and during pregnancy), lymphoma -primary central nervous system, macroglobulinemia -Waldenstrom's, malignant fibrous histiocytoma of bone/osteosarcoma, medulloblastoma (childhood), melanoma, melanoma-intraocular (eye), Merkel cell carcinoma, mesothelioma (adult) malignant, mesothelioma (childhood), metastatic squamous neck cancer with occult primary, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia, chronic, myeloid leukemia (adult and childhood) acute, myeloma-multiple, myeloproliferative disorders-chronic, nasal cavity and paranasal sinus cancer, nasopharyngeal cancer, nasopharyngeal cancer (childhood), neuroblastoma, non-small cell lung cancer, oral cancer (childhood), oral cavity and lip cancer, oropharyngeal cancer, osteosarcoma/malignant fibrous histiocytoma of bone, ovarian cancer (childhood), ovarian epithelial cancer, ovarian germ cell tumor, ovarian low malignant potential tumor, pancreatic cancer, pancreatic cancer (childhood), pancreatic cancer -islet cell, paranasal sinus and nasal cavity cancer, parathyroid cancer, penile cancer, pheochromocytoma, pineoblastoma and supratentorial primitive neuroectodermal tumors (childhood), pituitary tumor, plasma cell neoplasm/multiple myeloma, pleuropulmonary blastoma, pregnancy and breast cancer, primary central nervous system lymphoma, prostate cancer, rectal cancer, renal cell (kidney) cancer, renal cell (kidney) cancer (childhood), renal pelvis and ureter-transitional cell cancer, retinoblastoma, rhabdomyosarcoma (childhood), salivary gland cancer, salivary gland cancer (childhood), sarcoma-Ewing's family of tumors, sarcoma-Kaposi's, sarcoma-soft tissue (adult and childhood), sarcoma-uterine, Sézary syndrome, skin cancer (non-melanoma), skin cancer (childhood), skin cancer (melanoma), skin carcinoma-Merkel cell, small cell lung cancer, small intestine cancer, soft tissue sarcoma (adult and childhood), squamous cell carcinoma, squamous neck cancer with occult primary-metastatic, stomach (gastric) cancer, stomach (gastric) cancer (childhood), supratentorial primitive neuroectodermal tumors (childhood), testicular cancer, thymoma (childhood), thymoma and thymic carcinoma, thyroid cancer, thyroid cancer (childhood), transitional cell cancer of the renal pelvis and ureter, trophoblastic tumor, gestational, ureter and renal pelvis -transitional cell cancer, urethral cancer, uterine cancer -endometrial, uterine sarcoma, vaginal cancer, visual pathway and hypothalamic glioma (childhood), vulvar cancer, Waldenstrom's macroglobulinemia, Wilms' tumor. In certain embodiments, methods and compositions provided herein may be used to treat pancreatic cancer. In specific embodiments, the tumor may be a solid tumor. In specific embodiments, the tumor may be melanoma.

In an aspect, the compositions provided herein comprise effector proteins or polynucleotides encoding effector proteins involved in anti-tumor immune response. Non-limiting examples of effector proteins include MYH9, MYH10, FASN, GSTP, VIM, CLTC, HSPA8, PKM, P4HB, TUBB, SLC25A13, FLNA, PFKFB2, HSPD1, HSPA5, XRCC5, XRCC6,. RNF31, MYL12B, MYL12A, HSPA9, GAPDH, ATP5B, HNRNPU, PFKFB3, RBM10, GSN, PRPF31, DYNC1H1, IFI16, IFI204, PARP1, PMEL, PNKP, SLC25A4, PDIA6, and RBCK1, APEX1, CHD8, GDAP1, GPHN, IP04, MAP3K9, NLRC5, OXA1L, and RHOF. An effector protein may be the full length protein or any fragment thereof. An effector protein may include the wild type polypeptide sequence or may include one or more insertions, deletions, substitutions, duplications, or any other mutations compared to the wild type polypeptide sequence. In some embodiments, the mutation is in a catalytic domain of an effector protein. In some embodiments, the mutation impacts post translational modification of the effector protein.

In some embodiments, compositions described herein may include a cytokine. In some embodiments, the composition includes an IFI16 protein or a polynucleotide encoding the IFI16 protein. Gamma-interferon-inducible protein Ifi-16 (IFI16) also known as interferon-inducible myeloid differentiation transcriptional activator is a protein that in humans is encoded by the IFI16 gene involved in p53 modulation, Ras/Raf signaling pathway, and cell growth regulation, and programmed cell death. The IFI16 protein may be the wild type protein, or may include one or more mutations. In some embodiments, the mutation is at an amino acid residue that is subject to post translational modification, for example, phosphorylation, glycosylation, methylation, N-acetylation, S-nitrosylation, ubiquitination, or lipidation. Post translational modification may affect the activity, stability, and function of a protein or a protein complex. In some embodiments, composition described herein may include an IFI16 protein with mutations affecting post translational modification of the protein. In some embodiments, the mutation is at an Arg - Gly motif (RG motif). In some embodiments, the mutation is at an Arg (R) residue. The mutation may reduce methylation of the IFI16 protein by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% compared to wild type IFI16 protein. In some embodiments, the mutations include R-to-X mutations at amino acid residues corresponding to position 12 and/or positon 538 of SEQ ID NO: 3, where X is any amino acid residue. In some embodiments, X is a Cys (C) residue. In some embodiments, X is an Ala (A) residue.

In some embodiments, the composition includes an IFI204 protein or a polynucleotide encoding the IFI204 protein. IFI204 has been reported to be essential for IFN-production in macrophages. As described herein, the IFI204 protein may be the wild type protein, or may include one or more mutations. In some embodiments, the mutation is at an amino acid residue that is subject to post translational modification, for example, phosphorylation, glycosylation, methylation, N-acetylation, S-nitrosylation, ubiquitination, or lipidation. Post translational modification may affect the activity, stability, and function of a protein or a protein complex. In some embodiments, composition described herein may include an IFI204 protein with mutations affecting post translational modification of the protein. In some embodiments, the mutation is at an Arg-Gly motif (RG motif). In some embodiments, the mutation is at an Arg (R) residue. The mutation may reduce methylation of the IFI204 protein by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% compared to wild type IFI204 protein. In some embodiments, the mutations include R-to-X mutations at amino acid residues corresponding to position 12 and/or positon 538 of SEQ ID NO: 3, where X is any amino acid residue. In some embodiments, X is a Cys (C) residue. In some embodiments, X is an Ala (A) residue.

In some embodiments, the compositions provided herein are capable of regulating antigen presentation in a subject. For example, the compositions provided herein may be capable of regulating MHCI antigen presentation in a subject. In some embodiments, the compositions provided herein may be capable of regulating protein expression or activity involved in interaction with SHARPIN. In some embodiments, the composition provided herein includes an NLRCS protein or a polynucleotide encoding the NLRCS protein. In some embodiments, the composition provided herein is capable of inducing innate immune response in a cell. For example, the composition provided herein may be able to induce anti-microbial immunity in a cell. In some embodiments, the composition provided herein induces the cyclic guanosine-monophosphate adenosine-monophosphate synthase (cGAS) and stimulator of interferon genes (STING) response. In some embodiments, the composition provided herein induces inflammatory response and inflammasome assembly.

In an aspect, the compositions provided herein are capable of enhancing intrinsic immune response against tumor cells. The compositions provided herein may regulate the expression, activity, and/or function of effector proteins involved in antitumor immune response. For example, the compositions may increase the expression or activity of effector proteins or complexes involved in antitumor immune response. For example, the composition may decrease the expression or activity of effector proteins or complexes involved in antitumor immune response. In some embodiments, the composition increases expression or activity of a regulating of an effector protein involved in antitumor immune response. In some embodiments, the composition decreases the expression or activity of a regulator of an effector protein involved in antitumor immune response. Non-limiting examples of effector proteins include MYH9, MYH10, FASN, GSTP, VIM, CLTC, HSPA8, PKM, P4HB, TUBB, SLC25A13, FLNA, PFKFB2, HSPD1, HSPA5, XRCC5, XRCC6,. RNF31, MYL12B, MYL12A, HSPA9, GAPDH, ATP5B, HNRNPU, PFKFB3, RBM10, GSN, PRPF31, DYNC1H1, IFI16, IFI204, PARP1, PMEL, PNKP, SLC25A4, PDIA6, and RBCK1, APEX1, CHD8, GDAP1, GPHN, IP04, MAP3K9, NLRC5, OXA1L, and RHOF.

In some embodiments, the compositions provided herein regulate the expression or activity of a PRMT5 protein or a polynucleotide encoding the PRMT5 protein. For example, expression and/or activity of the PRMT5 protein or the polynucleotide encoding the PRMT5 protein may be increased or decreased. The increase or decrease may be on the transcript level, the protein level, or the post translation modification level. The increase or decrease may be on the gene level by genome editing. In some embodiments, the expression of the PRMT5 protein in a cell is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell. In some embodiments, the activity of the PRMT5 protein in a cell is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell.

In some embodiments, the compositions provided herein regulate the expression or activity of a MTAB protein or a polynucleotide encoding the MTAB protein. For example, expression and/or activity of the MTAB protein or the polynucleotide encoding the MTAB protein may be increased or decreased. The increase or decrease may be on the transcript level, the protein level, or the post translation modification level. The increase or decrease may be on the gene level by genome editing. In some embodiments, the expression of the MTAB protein in a cell is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell. In some embodiments, the activity of the MTAB protein in a cell is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell.

In some embodiments, the compositions provided herein regulate the expression or activity of a SHARPIN protein or a polynucleotide encoding the SHARPIN protein. For example, expression and/or activity of the SHARPIN protein or the polynucleotide encoding the SHARPIN protein may be increased or decreased. The increase or decrease may be on the transcript level, the protein level, or the post translation modification level. The increase or decrease may be on the gene level by genome editing. In some embodiments, the expression of the SHARPIN protein in a cell is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell. In some embodiments, the activity of the SHARPIN protein in a cell is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell.

In an aspect, provided herein are compositions that regulate expression and/or activity of effector proteins, for example, effector proteins involved in innate immune response, anti-microbial response, inflammatory pathways, and/or apoptosis pathways. Non-limiting examples of effector proteins include MYH9, MYH10, FASN, GSTP, VIM, CLTC, HSPA8, PKM, P4HB, TUBB, SLC25A13, FLNA, PFKFB2, HSPD1, HSPA5, XRCC5, XRCC6, RNF31, MYL12B, MYL12A, HSPA9, GAPDH, ATP5B, HNRNPU, PFKFB3, RBM10, GSN, PRPF31, DYNC1H1, IFI16, IFI204, PARP1, PMEL, PNKP, SLC25A4, PDIA6, and RBCK1, APEX1, CHD8, GDAP1, GPHN, IPO4, MAP3K9, NLRC5, OXA1L, and RHOF. In preferred embodiments, the effector protein may be a NLRC5 protein, an IFI204 protein, or an IFI16 protein.

Compositions provided herein may include regulating agents that regulate expression or activity of effector proteins. The regulating agents may include polypeptides, fusion proteins, polynucleotides, or any combination thereof. The regulating agents may include protein complexes, protein-nucleic acid complexes, ribonucleoprteins (RNPs), or any combination thereof. In some embodiments, the regulating agent increases expression or activity of the effector protein. The regulating agent may include a polypeptide comprising a functional domain. In some embodiments, the functional domain may be an expression activation domain, or a polynucleotide encoding an expression activation domain. non-limiting examples of expression activation domains include VP16, VP64, p65, VP128, p300 catalytic domain, TET1 catalytic domain, TDG, Ldbl self-associated domain, SAM activator (VP64, p65, HSF1), VPR (VP64, p65, Rta). In some aspects, the repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, MeCP2, or any combination thereof. In some embodiments, the functional domain may be a repression domain, a methylation domain, a de-methylation domain, a methyltransferase domain, a deaminase domain, a histone acetyltransferase domain, a histone deacetylase domain, or any fragment thereof.

The regulating agent may include a DNA targeting polypeptide. In some embodiments, the DNA targeting polypeptide includes a zinc finger domain. In some embodiments, the DNA targeting polypeptide includes a transcription activator like effector (TALE) repeat domain. In some embodiments, the regulating agent comprises a nucleic acid-guided protein complexed with a guide nucleic acid that recognize specific polynucleotide sequences in a cell. In some embodiments, the nucleic acid is a guide RNA. In some embodiments, the inhibitory agent comprises a RNA-guided CRISPR/Cas protein. In some embodiments, the CRISPR/Cas protein is type II CRISPR/Cas protein, a type V CRISPR/Cas protein, a type VII CRISPR/Cas protein, Cas9, CasX, CasY, Cpfl, C2c1, C2c2, or C2c3, or other CRISPR/Cas proteins. In some embodiments, the CRISPR/Cas protein has reduced nuclease activity. In some embodiments, the CRISPR/Cas protein is a nickase. In some embodiments, the CRISPR/Cas protein has no nuclease activity. In some embodiments, the CRISPR/Cas protein is a Cas9 protein. in some embodiments, the Cas9 protein comprises one or more mutations that reduces nuclease activity. In preferred embodiments, the one or more mutations are D10A and/or H840A of the wild type. In some embodiments, the polynucleotide comprises a RNA sequence that is reverse complementary to a polynucleotide that encodes a NLRC5 protein, an IFI16 protein, or an IFI204 protein in a cell.

In some embodiments, the compositions provided herein regulate the expression or activity of an NLRC5 protein or a polynucleotide encoding the NLRC5 protein. For example, expression and/or activity of the NLRC5 protein or the polynucleotide encoding the NLRC5 protein may be increased or decreased. The increase or decrease may be on the transcript level, the protein level, or the post translation modification level. The increase or decrease may be on the gene level by genome editing. In some embodiments, the expression of the NLRC5 protein in a cell is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, or at least about 350% compared to a control cell.

It should be appreciated NLRCS protein and homologs useful in the present application would be apparent to the skilled artisan and are within the scope of this disclosure. An exemplary NLRCS protein is provided below:

> Q86WI3-1 Protein NLRC5.
(SEQ ID NO: 1)
MDPVGLQLGNKNLWSCLVRLLTKDPEWLNAKMKFFLPNTDLDSRNETLDPE
QRVILQLNKLHVQGSDTWQSFIHCVCMQLEVPLDLEVLLLSTFGYDDGFTS
QLGAEGKSQPESQLHHGLKRPHQSCGSSPRRKQCKKQQLELAKKYLQLLRT
SAQQRYRSQIPGSGQPHAFHQVYVPPILRRATASLDTPEGAIMGDVKVEDG
ADVSISDLFNTRVNKGPRVTVLLGKAGMGKTTLAHRLCQKWAEGHLNCFQA
LFLFEFRQLNLITRFLTPSELLFDLYLSPESDHDTVFQYLEKNADQVLLIF
DGLDEALQPMGPDGPGPVLTLFSHLCNGTLLPGCRVMATSRPGKLPACLPA
EAAMVHMLGFDGPRVEEYVNHFFSAQPSREGALVELQTNGRLRSLCAVPAL
CQVACLCLHHLLPDHAPGQSVALLPNMTQLYMQMVLALSPPGHLPTSSLLD
LGEVALRGLETGKVIFYAKDIAPPLIAFGATHSLLTSFCVCTGPGHQQTGY
AFTHLSLQEFLAALHLMASPKVNKDTLTQYVTLHSRWVQRTKARLGLSDHL
PTFLAGLASCTCRPFLSHLAQGNEDCVGAKQAAVVQVLKKLATRKLTGPKV
VELCHCVDETQEPELASLTAQSLPYQLPFHNFPLTCTDLATLTNILEHREA
PIHLDFDGCPLEPHCPEALVGCGQIENLSFKSRKCGDAFAEALSRSLPTMG
RLQMLGLAGSKITARGISHLVKALPLCPQLKEVSFRDNQLSDQVVLNIVEV
LPHLPRLRKLDLSSNSICVSTLLCLARVAVTCPTVRMLQAREADLIFLLSP
PTETTAELQRAPDLQESDGQRKGAQSRSLTLRLQKCQLQVHDAEALIALLQ
EGPHLEEVDLSGNQLEDEGCRLMAEAASQLHIARKLDLSNNGLSVAGVHCV
LRAVSACWTLAELHISLQHKTVIFMFAQEPEEQKGPQERAAFLDSLMLQMP
SELPLSSRRMRLTHCGLQEKHLEQLCKALGGSCHLGHLHLDFSGNALGDEG
AARLAQLLPGLGALQSLNLSENGLSLDAVLGLVRCFSTLQWLFRLDISFES
QHILLRGDKTSRDMWATGSLPDFPAAAKFLGFRQRCIPRSLCLSECPLEPP
SLTRLCATLKDCPGPLELQLSCEFLSDQSLETLLDCLPQLPQLSLLQLSQT
GLSPKSPFLLANTLSLCPRVKKVDLRSLHHATLHFRSNEEEEGVCCGRFTG
CSLSQEHVESLCWLLSKCKDLSQVDLSANLLGDSGLRCLLECLPQVPISGL
LDLSHNSISQESALYLLETLPSCPRVREASVNLGSEQSFRIHFSREDQAGK
TLRLSECSFRPEHVSRLATGLSKSLQLTELTLTQCCLGQKQLAILLSLVGR
PAGLFSLRVQEPWADRARVLSLLEVCAQASGSVTEISISETQQQLCVQLEF
PRQEENPEAVALRLAHCDLGAHHSLLVGQLMETCARLQQLSLSQVNLCEDD
DASSLLLQSLLLSLSELKTFRLTSSCVSTEGLAHLASGLGHCHHLEELDLS
NNQFDEEGTKALMRALEGKWMLKRLDLSHLLLNSSTLALLTHRLSQMTCLQ
SLRLNRNSIGDVGCCHLSEALRAATSLEELDLSHNQIGDAGVQHLATILPG
LPELRKIDLSGNSISSAGGVQLAESLVLCRRLEELMLGCNALGDPTALGLA
QELPQHLRVLHLPFSHLGPGGALSLAQALDGSPHLEEISLAENNLAGGVLR
FCMELPLLRQIDLVSCKIDNQTAKLLTSSFTSCPALEVILLSWNLLGDEAA
AELAQVLPQMGRLKRVDLEKNQITALGAWLLAEGLAQGSSIQVIRLWNNPI
PCDMAQHLKSQEPRLDFAFFDNQPQAPWGT.

The NLRC5 protein and use in cancer therapy as disclosed in US201703212851 and its entire content is hereby incorporated by reference.

In some embodiments, the compositions provided herein regulate the expression or activity of an IFI16 protein or a polynucleotide encoding the IFI16 protein. For example, expression and/or activity of the IFI16 protein or the polynucleotide encoding the IFI16 protein may be increased or decreased. The increase or decrease may be on the transcript level, the protein level, or the post translation modification level. The increase or decrease may be on the gene level by genome editing. In some embodiments, the expression of the IFI16 protein in a cell is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, or at least about 350% compared to a control cell.

In some embodiments, the activity of the IFI16 protein in a cell is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, or at least about 350% compared to a control cell.

In some embodiments, the expression of the IFI16 protein is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell.

In some embodiments, the activity of the IFI16 protein is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell.

The IFI16 gene encodes a transcription and cell cycle regulation protein. It should be appreciated IFI16 protein and homologs useful in the present application would be apparent to the skilled artisan and are within the scope of this disclosure. An exemplary IFI16 polypeptide sequence is provided below:

> Gamma-interferon-inducible protein 16
(SEQ ID NO: 2)
MGKKYKNIVLLKGLEVINDYHFRMVKSLLSNDLKLNLKMREEYDKIQIADL
MEEKFRGDAGLGKLIKIFEDIPTLEDLAETLKKEKLKVKGPALSRKRKKEV
DATSPAPSTSSTVKTEGAEATPGAQKRKKSTKEKAGPKGSKVSEEQTQPPS
PAGAGMSTAMGRSPSPKTSLSAPPNSSSTENPKTVAKCQVTPRRNVLQKRP
VIVKVLSTTKPFEYETPEMEKKIMFHATVATQTQFFHVKVLNTSLKEKFNG
TKKIIIISDYLEYDSLLEVNEESTVSEAGPNQFEVPNKIINRAKETLKIDI
LHKQASGNIVYGVFMLHKKTVNQKTTIYEIQDDRGKMDVVGTGQCHNIPCE
EGDKLQLFCFRLRKKNQMSKLISEMHSFIQIKKKTNPRNNDPKSMKLPQEQ
RQLPYPSEASTTFPESHLRTPQMPPTTPSSSFFTKKSEDTISKMNDFMRMQ
ILKEGSHFPGPFMTSIGPAESHPHTPQMPPSTPSSSFLTTKSEDTISKMND
FMRMQILKEGSHFPGPFMTSIGPAESHPHTPQMPPSTPSSSFLTTLKPRLK
TEPEEVSIEDSAQSDLKEVMVLNATESFVYEPKEQKKMFHATVATENEVFR
VKVFNIDLKEKFTPKKIIAIANYVCRNGFLEVYPFTLVADVNADRNMEIPK
GLIRSASVTPKINQLCSQTKGSFVNGVFEVHKKNVRGEFTYYEIQDNTGKM
EVVVHGRLTTINCEEGDKLKLTCFELAPKSGNTGELRSVIHSHIKVIKTRK
NKKDILNPDSSMETSPDFFF

In some embodiments, the compositions provided herein regulate the expression or activity of an IFI204 protein or a polynucleotide encoding the IFI204 protein. For example, expression and/or activity of the IFI204 protein or the polynucleotide encoding the IFI204 protein may be increased or decreased. The increase or decrease may be on the transcript level, the protein level, or the post translation modification level. The increase or decrease may be on the gene level by genome editing. In some embodiments, the expression of the IFI204 protein in a cell is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, or at least about 350% compared to a control cell.

In some embodiments, the activity of the IFI204 protein in a cell is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, or at least about 350% compared to a control cell.

In some embodiments, the expression of the IFI204 protein is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell.

In some embodiments, the activity of the IFI204 protein is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell.

The IFI204 gene encodes a transcription and cell cycle regulation protein. It should be appreciated IFI204 protein and homologs useful in the present application would be apparent to the skilled artisan and are within the scope of this disclosure. Exemplary IFI204 polypeptide sequences are provided below:

> Interferon-activable protein 204 (Mus musculus)
(SEQ ID NO: 3)
MVNEYKRIVLLRGLECINKHYFSLFKSLLARDLNLERDNQEQYTTIQIANM
MEEKFPADSGLGKLIAFCEEVPALRKRAEILKKERSEVTGETSLEKNGQEA
GPATPTSTTSHMLASERGETSATQEETSTAQAGTSTAQARTSTAQAGTSTA
QKRKIMREEETGVKKSKAAKEPDQPPCCEEPTARCQSPILHSSSSASSNIP
SAKNQKSQPQNQNIPRGAVLHSEPLTVMVLTATDPFEYESPEHEVKNMLHA
TVATVSQYFHVKVFNINLKEKFTKKNFIIISNYFESKGILEINETSSVLEA
APDQMIEVPNSIIRNANASPKICDIQKGTSGAVFYGVFTLHKKTVNRKNTI
YEIKDGSGSIEVVGSGKWHNINCKEGDKLHLFCFHLKTIDRQPKLVCGEHS
FIKISKRGNVPKEPAKEEDHHHGPKQVMVLKVTEPFTYDLKEDKRMFHATV
ATETEFFRVKVFDTALKSKFIPRNIIAISDYFGCNGFLEIYRASCVSDVNV
NPTMVISNTLRQRANATPKISYLFSQARGTFVSGEYLVNKKTERNKFIYYG
IGDDTGKMEVVVYGRLTNVRCEPGSKLRLVCFELTSTEDGWQLRSVRHSYM
QVINARK
> Interferon-activable protein 204 (Mus musculus)
(SEQ ID NO: 4)
MVNEYKRIVLLAGLECINKHYFSLFKSLLARDLNLERDNQEQYTTIQIANM
MEEKFPADSGLGKLIAFCEEVPALRKRAEILKKERSEVTGETSLEKNGQEA
GPATPTSTTSHMLASERGETSATQEETSTAQAGTSTAQARTSTAQAGTSTA
QKRKIMREEETGVKKSKAAKEPDQPPCCEEPTARCQSPILHSSSSASSNIP
SAKNQKSQPQNQNIPRGAVLHSEPLTVMVLTATDPFEYESPEHEVKNMLHA
TVATVSQYFHVKVFNINLKEKFTKKNFIIISNYFESKGILEINETSSVLEA
APDQMIEVPNSIIRNANASPKICDIQKGTSGAVFYGVFTLHKKTVNRKNTI
YEIKDGSGSIEVVGSGKWHNINCKEGDKLHLFCFHLKTIDRQPKLVCGEHS
FIKISKRGNVPKEPAKEEDHHHGPKQVMVLKVTEPFTYDLKEDKRMFHATV
ATETEFFRVKVFDTALKSKFIPRNIIAISDYFGCNGFLEIYRASCVSDVNV
NPTMVISNTLRQRANATPKISYLFSQARGTFVSGEYLVNKKTERNKFIYYG
IGDDTGKMEVVVYGRLTNVRCEPGSKLRLVCFELTSTEDGWQLRSVRHSYM
QVINARK
> Interferon-activable protein 204 (Mus musculus)
(SEQ ID NO: 5)
MVNEYKRIVLLRGLECINKHYFSLFKSLLARDLNLERDNQEQYTTIQIANM
MEEKFPADSGLGKLIAFCEEVPALRKRAEILKKERSEVTGETSLEKNGQEA
GPATPTSTTSHMLASERGETSATQEETSTAQAGTSTAQARTSTAQAGTSTA
QKRKIMREEETGVKKSKAAKEPDQPPCCEEPTARCQSPILHSSSSASSNIP
SAKNQKSQPQNQNIPRGAVLHSEPLTVMVLTATDPFEYESPEHEVKNMLHA
TVATVSQYFHVKVFNINLKEKFTKKNFIIISNYFESKGILEINETSSVLEA
APDQMIEVPNSIIRNANASPKICDIQKGTSGAVFYGVFTLHKKTVNRKNTI
YEIKDGSGSIEVVGSGKWHNINCKEGDKLHLFCFHLKTIDRQPKLVCGEHS
FIKISKRGNVPKEPAKEEDHHHGPKQVMVLKVTEPFTYDLKEDKRMFHATV
ATETEFFRVKVFDTALKSKFIPRNIIAISDYFGCNGFLEIYRASCVSDVNV
NPTMVISNTLRQRANATPKISYLFSQAAGTFVSGEYLVNKKTERNKFIYYG
IGDDTGKMEVVVYGRLTNVRCEPGSKLRLVCFELTSTEDGWQLRSVRHSYM
QVINARK
> Interferon-activable protein 204 (Mus musculus)
(SEQ ID NO: 6)
MVNEYKRIVLLAGLECINKHYFSLFKSLLARDLNLERDNQEQYTTIQIANM
MEEKFPADSGLGKLIAFCEEVPALRKRAEILKKERSEVTGETSLEKNGQEA
GPATPTSTTSHMLASERGETSATQEETSTAQAGTSTAQARTSTAQAGTSTA
QKRKIMREEETGVKKSKAAKEPDQPPCCEEPTARCQSPILHSSSSASSNIP
SAKNQKSQPQNQNIPRGAVLHSEPLTVMVLTATDPFEYESPEHEVKNMLHA
TVATVSQYFHVKVFNINLKEKFTKKNFIIISNYFESKGILEINETSSVLEA
APDQMIEVPNSIIRNANASPKICDIQKGTSGAVFYGVFTLHKKTVNRKNTI
YEIKDGSGSIEVVGSGKWHNINCKEGDKLHLFCFHLKTIDRQPKLVCGEHS
FIKISKRGNVPKEPAKEEDHHHGPKQVMVLKVTEPFTYDLKEDKRMFHATV
ATETEFFRVKVFDTALKSKFIPRNIIAISDYFGCNGFLEIYRASCVSDVNV
NPTMVISNTLRQRANATPKISYLFSQAAGTFVSGEYLVNKKTERNKFIYYG
IGDDTGKMEVVVYGRLTNVRCEPGSKLRLVCFELTSTEDGWQLRSVRHSYM
QVINARK

In an aspect, the compositions provided herein is capable of regulating post translational modification of one or more proteins involved in anti tumor immune responses. In some embodiments, the post translational modification regulates chemokine production and interferon production. For instance, the composition provided herein may regulate methylation of the cGAS/STING complex. In some embodiments, the composition provided herein regulates methylation of IFI16 in the cGAS/STING complex in a cell. In some embodiments, the composition provided herein increases methylation level of IFI16 in the cGAS/STING complex in the cell. In some embodiments, the composition provided herein decreases methylation level of IFI16 in the cGAS/STING complex in the cell. In some embodiments, the methylation level of IFI16 in the cell is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell.

In an aspect, the compositions provided herein is capable of regulating post translational modification of one or more proteins involved in anti tumor immune responses. In some embodiments, the post translational modification regulates chemokine production and interferon production. For instance, the composition provided herein may regulate methylation of the cGAS/STING complex. In some embodiments, the composition provided herein regulates methylation of IFI16 in the cGAS/STING complex in a cell. In some embodiments, the composition provided herein increases methylation level of IFI204 in the cGAS/STING complex in the cell. In some embodiments, the composition provided herein decreases methylation level of IFI204 in the cGAS/STING complex in the cell. In some embodiments, the methylation level of IFI204 in the cell is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% compared to a control cell.

### Checkpoint Inhibitors

Immune checkpoint therapy (ICT) using checkpoint inhibitor has been used for efficient treatment of certain tumors. With other tumor types, the efficiency of ICT may be diminished by ICT resistance, including adaptive resistance to therapy due to loss of tumor antigenicity and reduced immune cell infiltration and activation.

However, many patients do not respond to these treatments when administered alone. For example, at least one study indicated that 70-80% of subjects receiving anti-PD-1 therapy were non-responders. Additionally, adaptive immune resistance, whereby immune-checkpoint ligands such as PD-L1 are induced in tumors in response to an endogenous antitumor immune response leading to immune exhaustion, suggests that immune checkpoint inhibition as a monotherapy will only succeed in the setting of a pre-existing, chronically inflamed and exhausted antitumor immune response in the patient. Thus, expanded efficacy might be achieved when immune checkpoint inhibition is combined with another therapy that induces a de novo antitumor immune response.

Accordingly, provided herein are compositions and methods for enhanced efficacy of ICT treatment.

Non-limiting examples of checkpoint inhibitors include inhibitors that target CTLA4, PD1, PDL1, LAG3, B7.1, B7-H3, B7-H4, TIM3, VISTA, CD137, OX-40, CD40, CD27, CCR4, GITR, NKG2D, KIR. IL-12, or any combination thereof. The checkpoint inhibitors may be antibodies, fusion proteins, compounds, nucleic acids, or small molecules. Non-limiting examples of checkpoint inhibitors in development include ipilimumab, tremelimumab, galiximab, MDX-1106, BMS-936558, MEDI4736, MPDL3280A, MEDI6469, BMS-986016, BMS-663513, PF-05082566, IPH2101, KW-0761, CDX-1127, CP-870, CP-893, GSK2831781, MSB0010718C, MK3475, CT-011, AMP-224, MDX-1105, IMP321, and MGA271. Additionally, ICT therapies that target other immune checkpoint proteins that are either not disclosed or have not yet been discovered are also contemplated for the purposes of the disclosure.

Provided herein are combination therapy and uses for treatment and amelioration of symptoms of cancer. Any effector proteins, inhibitory agents, and other agents may be used in combination with checkpoint inhibitors for enhanced ICT treatment. In particular, the combination therapy provided herein may be used to overcome resistance ICT by improving infiltration of lymphoid cells and enhancing tumor cell recognition by the immune system. ICT inhibitors and effector proteins or inhibitory agents provided herein can be administered to a subject thereof at any suitable time point before, during, or after the ICI, or any combination thereof. In certain instances, the subject may be immune competent. In certain instances, the subject may be immune compromised. In certain aspects, provided herein are methods for treatment of tumor with at least one checkpoint inhibitor and at least one PRMT5 inhibitory agent, at least one effector protein, or any combination thereof. In preferred embodiments, the combinatorial treatment is more effective in treating cancer compared to ICT treatment alone. In certain embodiments, the combinatorial treatment results in reduction of tumor size. In preferred embodiments, the combinatorial treatment results in reduction of tumor size at least 10%, at least 15%, at least 30%, at least 50%, at least 75%, at least 90%, or at least 100% in size. In further preferred embodiments, the combinatorial treatment results in reduction of tumor size of at least 10%, at least 15%, at least 30%, at least 50%, at least 75%, at least 90%, or at least 100% more compared to ICT treatment.

In additional aspects, compositions provided herein may be used in combination with adoptive T cell therapy, chimeric antigen receptor T cell therapy, natural killer (NK) cell therapy, dendritic cell therapy, other immune cell therapy, radiotherapy, chemotherapy, gene therapy, or any combination thereof. The immune cells may be naturally occurring or modified. Methods and uses of cancer therapies are known to those skilled in the art.

### PR1VIT5 Inhibitory Agents

Provided herein are compositions and methods for inhibition or reduction of expression and/or activity of PRMT5 protein in a cell. Inhibitors or inhibitory agents may be used to inhibit gene expression or protein function and activity. The inhibition may be complete inhibition or partial inhibition. Inhibition can be a result of direct or indirect inhibition meaning the inhibitors acts directly on the target (protein or gene to be inhibited) or the inhibitor can act indirectly via a different protein or gene upstream of the target. An inhibitor may be a small molecule, a compound, a protein, a nucleic acid, a vector, or a nucleic acid-protein complex.

The expression and/or activity of PRMT5 may be determined by methods known to those skilled in the art. Gene or protein expression and activity may be accessed and described by any suitable metric. For example, the expression of PRMT5 may be described by the number of cells expressing PRMT5, the number of cells expressing PRMT5 at a certain level, the fraction of cells expressing PRMT5 at a certain level, the level of PRMT5 in certain cells, or any combination thereof. Methods of determining gene expression and protein levels are known to those skilled in the art.

Provided herein are methods and compositions for treatment and inhibition of progression of cancer comprising administering to a subject in need thereof a therapeutically effective amount of an inhibitory agent of PRMT5 protein and/or a polynucleotide encoding the PRMT5 protein. In some embodiments, the inhibitory agent comprises a protein comprising a nucleotide recognition domain, e.g. a DNA recognition domain. In some embodiments, the inhibitory agent comprises a protein comprising a nucleotide recognition domain, e.g. a DNA recognition domain, and an effector domain. In some embodiments, the effector domain is a transcriptional activator domain, transcriptional repressor domain, DNA methyl transferase domain, DNA demethylase domain, histone acetyltransferase domain, histone deacetylase domain, and combinations thereof. In some embodiments, the nucleotide recognition domain is derived from, or homologous to, a transcription activator like effector (TALE) DNA recognition domain. In some embodiments, the nucleotide recognition domain is derived from, or homologous to a zinc finger DNA recognition domain. In some embodiments, the nucleotide recognition domain is derived from, or homologous to a helix-turn-helix domain, a leucine zipper domain, a winged helix domain, a CRISPR/Cas protein DNA binding domain, a Wor3 domain, a HMG box, a OB fold domain, or any combination thereof In some embodiments, the nucleotide recognition domain recognizes and binds to a sequence in a polynucleotide that encodes a PRMT5 protein in a cell. In some embodiments, the polynucleotide is a DNA. In some embodiments, the nucleotide recognition domain recognizes and binds to a sequence in a polynucleotide that encodes a SHARPIN protein in a cell. In some embodiments, the nucleotide recognition domain recognizes and binds to a sequence in a polynucleotide that encodes a MTAP protein in a cell.

In some embodiments, the decrease in expression of PRMT5 comprises a decrease of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, or more in a treated with the method and composition described herein compared to expression of PRMT5 in a control cell. In some embodiments, the decrease in expression of PRMT5 comprises a decrease of about 20% to about 100%, about 50% to about 100%, about 20% to about 50%, at least about 20%, at least about 50%, compared to expression of PRMT5 in a control cell.

In some embodiments, the decrease in expression of an MTAP protein comprises a decrease of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, or more in a treated with the method and composition described herein compared to expression of the MTAP in a control cell. In some embodiments, the decrease in expression of the MTAP comprises a decrease of about 20% to about 100%, about 50% to about 100%, about 20% to about 50%, at least about 20%, at least about 50%, compared to expression of MTAP in a control cell.

In some embodiments, the decrease in expression of an SHARPIN protein comprises a decrease of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, or more in a treated with the method and composition described herein compared to expression of the SHARPIN in a control cell. In some embodiments, the decrease in expression of the SHARPIN comprises a decrease of about 20% to about 100%, about 50% to about 100%, about 20% to about 50%, at least about 20%, at least about 50%, compared to expression of SHARPIN in a control cell.

Provided herein are methods and compositions for treatment and inhibition of progression of cancer, comprising administering to a subject in need thereof a therapeutically effective amount of at least one, at least two, at least three, at least four, at least five, at least six, at least seven, at least eight, at least nine, or at least ten inhibitory agents. In some embodiments, one or more inhibitory agents comprise a protein comprising a nucleotide recognition domain, e.g. a DNA recognition domain. In some embodiments, one or more inhibitory agents comprise a protein comprising a nucleotide recognition domain, e.g. a DNA recognition domain, and an effector domain. In some embodiments, the effector domain is a transcriptional activator domain, DNA demethylase domain, histone deacetylase domain, and combinations thereof. In some embodiments, the nucleotide recognition domain is derived from, or homologous to, a transcription activator like effector (TALE) DNA recognition domain. In some embodiments, the nucleotide recognition domain is derived from, or homologous to a zinc finger DNA recognition domain. In some embodiments, the nucleotide recognition domain is derived from, or homologous to a helix-turn-helix domain, a leucine zipper domain, a winged helix domain, a Wor3 domain, a HMG box, an OB fold domain, or any combination thereof.

Therapeutic approaches based on siRNAs and microRNAs are available using methods well known to those skilled in the art. For example, a synthetic siRNA can be introduced into the target cells to elicit RNA interference (RNAi), thereby inhibiting the expression of a specific messenger RNA (mRNA) to produce a gene silencing effect. For example, single stranded RNAs acting as microRNA antagonists (also known as antagomirs or anti-miRs) can be introduced to inhibit the action of the endogenous miRNAs. In the replacement approach, synthetic miRNAs (also known as miRNA mimics) can be introduced to mimic the function of the endogenous miRNAs.

In some embodiments, the inhibitory agent comprises a protein. In some embodiments, the inhibitory agent comprises a protease that catalyzes cleavage of a PRMT5 protein. In some embodiments, the inhibitory agent comprises a nuclease that catalyzes cleavage of a polynucleotide. In some embodiments, the inhibitory agent comprises a nuclease that catalyzes cleavage of a polynucleotide that encodes a PRMT5 protein in a cell. In some embodiments, the inhibitory agent comprises a nuclease that catalyzes cleavage of a polynucleotide in a cell that does not involve insertion, deletion, substitution, frameshifting, or other genome editing events in the genome of the cell. Non-limiting examples of nucleases include zinc finger nuclease, fokl nuclease, TALEN nucleases, meganuclease, Cas proteins. In some embodiments, the nuclease is a Cas9 nuclease. In some embodiments, the nuclease is a C2c2 nuclease.

In some embodiments, the inhibitory agent comprises a nucleic acid-guided protein complexed with a guide nucleic acid that recognize specific polynucleotide sequences in a cell. In some embodiments, the nucleic acid is a guide RNA. In some embodiments, the inhibitory agent comprises a RNA-guided CRISPR/Cas protein. In some embodiments, the CRISPR/Cas protein is type II CRISPR/Cas protein, a type V CRISPR/Cas protein, a type VII CRISPR/Cas protein, Cas9, CasX, CasY, Cpf1, C2c1, C2c2, or C2c3, or other CRISPR/Cas proteins. In some embodiments, the polynucleotide comprises a RNA sequence that is reverse complementary to a polynucleotide that encodes a PRMT5 protein in a cell. In some embodiments, the guide RNA comprises a RNA sequence that is reverse complementary to a polynucleotide that encodes a PRMT5 protein in a cell. In a preferred embodiment, the CRISPR/Cas protein is C2c2.

In some embodiments, the inhibitory agent comprises a nucleic acid-guided protein complexed with a guide RNA that recognizes specific polynucleotide sequences in a cell. In some embodiments, the nucleic acid is a guide RNA. In some embodiments, the inhibitory agent comprises a RNA-guided CRISPR/Cas protein. In some embodiments, the CRISPR/Cas protein is type II CRISPR/Cas protein, a type V CRISPR/Cas protein, a type VII CRISPR/Cas protein, Cas9, CasX, CasY, Cpf1, C2c1, C2c2, or C2c3, or other CRISPR/Cas proteins. In some embodiments, the polynucleotide comprises a RNA sequence that is reverse complementary to a DNA that encodes a PRMT5 protein in a cell. In some embodiments, the guide RNA comprises a RNA sequence that is reverse complementary to a DNA that encodes a PRMT5 protein in a cell. In some embodiments, the CRISPR/Cas protein comprises a mutation in the nuclease domain. In some embodiments, the CRISPR/Cas protein comprises a mutation in the nuclease domain that reduces or abolishes the catalytic activity of the nuclease domain. In some embodiments, the CRISPR/Cas protein comprises a mutation in the nuclease domain that renders the nuclease domain a nickase domain. In some embodiments, the CRISPR/Cas protein is a Cas9 protein comprising mutations D10A and/or H840A compared to the wild type spCas9 protein. In some embodiments, the CRISPR/Cas protein lacks the HNH nuclease domain. In some embodiments, the CRISPR/Cas protein further comprises an effector domain. In certain embodiments, the effector domain is a transcriptional repressor domain, DNA methyl transferase domain, histone acetyltransferase domain, histone deacetylase domain, and combinations thereof

A guide nucleic acid (e.g., guide RNA) can bind to a Cas protein and target the Cas protein to a specific location within a target polynucleotide. A guide nucleic acid can comprise a nucleic acid-targeting segment and a Cas protein binding segment.

A guide nucleic acid can refer to a nucleic acid that can hybridize to another nucleic acid, for example, the target polynucleotide in the genome of a cell. A guide nucleic acid can be RNA, for example, a guide RNA. A guide nucleic acid can be DNA. A guide nucleic acid can comprise DNA and RNA. A guide nucleic acid can be single stranded. A guide nucleic acid can be double-stranded. A guide nucleic acid can comprise a nucleotide analog. A guide nucleic acid can comprise a modified nucleotide. The guide nucleic acid can be programmed or designed to bind to a sequence of nucleic acid site-specifically.

A guide nucleic acid can comprise one or more modifications to provide the nucleic acid with a new or enhanced feature. A guide nucleic acid can comprise a nucleic acid affinity tag. A guide nucleic acid can comprise synthetic nucleotide, synthetic nucleotide analog, nucleotide derivatives, and/or modified nucleotides.

The guide nucleic acid can comprise a nucleic acid-targeting region (e.g., a spacer region), for example, at or near the 5′ end or 3′ end, that is complementary to a protospacer sequence in a target polynucleotide. The spacer of a guide nucleic acid can interact with a protospacer in a sequence-specific manner via hybridization (base pairing). The protospacer sequence can be located 5′ or 3′ of protospacer adjacent motif (PAM) in the target polynucleotide. The nucleotide sequence of a spacer region can vary and determines the location within the target nucleic acid with which the guide nucleic acid can interact. The spacer region of a guide nucleic acid can be designed or modified to hybridize to any desired sequence within a target nucleic acid.

A guide nucleic acid can comprise two separate nucleic acid molecules, which can be referred to as a double guide nucleic acid. A guide nucleic acid can comprise a single nucleic acid molecule, which can be referred to as a single guide nucleic acid (e.g., sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like and tracrRNA-like molecules.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single- stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to target.

In some embodiments, the inhibitory agent comprises a RNA molecule. In some embodiments, the inhibitory agent comprises a non-coding RNA molecule. In some embodiments, the non-coding RNA molecule comprises a microRNA, an siRNA, an anti-sense RNA, or any combination thereof. In some embodiments, the polynucleotide comprises a RNA sequence that is reverse complementary to a DNA that encodes a PRMT5 protein in a cell. In some embodiments, the non-coding RNA comprises a siRNA that targets mRNA that encodes an PRMT5 protein. In some embodiments, the non-coding RNA comprises a siRNA that targets mRNA that encodes a SHARPIN protein or a MTAP protein. In some embodiments, the non-coding RNA comprises a microRNA that targets mRNA that encodes a PRMT5 protein. In some embodiments, the non-coding RNA comprises a microRNA that targets mRNA that encodes a SHARPIN protein, a MEK protein, or a MTAP protein.

In additional embodiments, the inhibitory agent may comprise other small or large molecules or compounds. PRMT5 inhibiting compounds as disclosed in PCT publications WO2015200677, WO2015200680, WO2017218802A1, WO2014128465, WO2014145214, WO2018065365, WO2018081451WO2018161922, WO2018167276, the entire contents of each are hereby incorporated by reference in their entirety.

### Therapeutic Approaches

In some embodiments, the compositions described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

A pharmaceutical composition can be a mixture of a composition or inhibitory agent described herein with one or more other chemical components (e.g. pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

The compositions described herein can be administered to the subject in a variety of ways, including intra-tumorally, parenterally, intramuscularly, colonically, rectally, intraperitoneally, intradermally, subcutaneously, intraperitoneally, or intravenously. In some embodiments, composition describe herein encompasses a small molecule. In some embodiments, the small molecule is an inhibitory agent or an inhibitor. Non-limiting examples of small molecules provided herein include gefitinib, sunitinib, dabrafenib, vemurafenib, trametinib, selumetinib, sorafnib, and Torin 1. The small molecule inhibitory agent or a pharmaceutically acceptable salt thereof may be administered by intratumoral intraperitoneal injection, intramuscular injection, subcutaneous injection, or intravenous injection of the subject. In some embodiments, the pharmaceutical compositions can be administered parenterally, intravenously, intramuscularly or orally. The oral agents comprising a small molecule inhibitory agent can be in any suitable form for oral administration, such as liquid, tablets, capsules, or the like. The oral formulations can be further coated or treated to prevent or reduce dissolution in stomach. The compositions of the present disclosure can be administered to a subject using any suitable methods known in the art. Suitable formulations for use in the present disclosure and methods of delivery are generally known in the art. For example, the small molecule inhibitory agent described herein can be formulated as pharmaceutical compositions with a pharmaceutically acceptable diluent, carrier or excipient. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

Pharmaceutical formulations described herein can be administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the pharmaceutical formulation is in the form of a tablet. In other embodiments, pharmaceutical formulations containing an composition or inhibitory agent described herein are in the form of a capsule. In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a composition or inhibitory agent described herein can be formulated for use as an aerosol, a mist or a powder. For buccal or sublingual administration, the compositions may take the form of tablets, lozenges, or gels formulated in a conventional manner. In some embodiments, a composition or inhibitory agent described herein can be prepared as transdermal dosage forms. In some embodiments, a composition or inhibitory agent described herein can be formulated into a pharmaceutical composition suitable for intramuscular, subcutaneous, or intravenous injection. In some embodiments, a composition or inhibitory agent described herein can be administered topically and can be formulated into a variety of topically administrable compositions, such as solutions, suspensions, lotions, gels, pastes, medicated sticks, balms, creams or ointments. In some embodiments, a composition or inhibitory agent described herein can be formulated in rectal compositions such as enemas, rectal gels, rectal foams, rectal aerosols, suppositories, jelly suppositories, or retention enemas.

### Biological Samples

A sample, e.g., a biological sample can be taken from a subject and examined to determine whether, for example, the subject produces an mRNA or a protein subject to regulation by the compositions provided herein and/or whether the subject produces a biomarker of tumor. A biological sample can comprise a plurality of biological samples. The plurality of biological samples can contain two or more biological samples; for examples, about 2-1000, 2-500, 2-250, 2-100, 2-75, 2-50, 2-25, 2-10, 10-1000, 10-500, 10-250, 10-100, 10-75, 10-50, 10-25, 25-1000, 25-500, 25-250, 25-100, 25-75, 25-50, 50-1000, 50-500, 50-250, 50-100, 50-75, 60-70, 100-1000, 100-500, 100-250, 250-1000, 250-500, 500-1000, 2, 3, 4, 5, 6, 7, 8, 9, 10, 11, 12, 13, 14, 15, 16, 17, 18, 19, 20, 21, 22, 23, 24, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 90, 95, 100, 110, 120, 130, 140, 150, 160, 170, 180, 190, 200, 210, 220, 230, 240, 250, 275, 300, 325, 350, 375, 400, 425, 450, 475, 500, 550, 600, 650, 700, 750, 800, 850, 900, 950, 1000, or more biological samples. The biological samples can be obtained from a plurality of subjects, giving a plurality of sets of a plurality of samples. The biological samples can be obtained from about 2 to about 1000 subjects, or more; for example, about 2-1000, 2-500, 2-250, 2-100, 2-50, 2-25, 2-20, 2-10, 10-1000, 10-500, 10-250, 10-100, 10-50, 10-25, 10-20, 15-20, 25-1000, 25-500, 25-250, 25-100, 25-50, 50-1000, 50-500, 50-250, 50-100, 100-1000, 100-500, 100-250, 250-1000, 250-500, 500-1000, or at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 68, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, 550, 600, 650, 700, 750, 800, 850, 900, 950, 1000 or more subjects.

The biological samples can be obtained from human subjects. The biological samples can be obtained from human subjects at different ages. The human subject can be prenatal (e.g., a fetus), a child (e.g., a neonate, an infant, a toddler, a preadolescent), an adolescent, a pubescent, or an adult (e.g., an early adult, a middle aged adult, a senior citizen). The human subject can be between about 0 months and about 120 years old, or older. The human subject can be between about 0 and about 12 months old; for example, about 1, 2, 3, 4, 5, 6, 7, 8, 9, 10, 11, or 12 months old. The human subject can be between about 0 and 12 years old; for example, between about 0 and 30 days old; between about 1 month and 12 months old; between about 1 year and 3 years old; between about 4 years and 5 years old; between about 4 years and 12 years old; about 1, 2, 3, 4, 5, 6, 7, 8, 9, 10, 11, or 12 years old. The human subject can be between about 13 years and 19 years old; for example, about 13, 14, 15, 16, 17, 18, or 19 years old. The human subject can be between about 20 and about 39 year old; for example, about 20, 21, 22, 23, 24, 25, 26, 27, 28, 29, 30, 31, 32, 33, 34, 35, 36, 37, 38, or 39 years old. The human subject can be between about 40 to about 59 years old; for example, about 40, 41, 42, 43, 44, 45, 46, 47, 48, 49, 50, 51, 52, 53, 54, 55, 56, 57, 58, or 59 years old. The human subject can be greater than 59 years old; for example, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 years old. The human subjects can include living subjects or deceased subjects. The human subjects can include male subjects and/or female subjects.

Biological samples can be obtained from any suitable source that allows determination of expression levels of genes, e.g., from cells, tissues, bodily fluids or secretions, or a gene expression product derived therefrom (e.g., nucleic acids, such as DNA or RNA; polypeptides, such as protein or protein fragments). The nature of the biological sample can depend upon the nature of the subject. If a biological sample is from a subject that is a unicellular organism or a multicellular organism with undifferentiated tissue, the biological sample can comprise cells, such as a sample of a cell culture, an excision of the organism, or the entire organism. If a biological sample is from a multicellular organism, the biological sample can be a tissue sample, a fluid sample, or a secretion.

The biological samples can be obtained from different tissues. The term tissue is meant to include ensembles of cells that are of a common developmental origin and have similar or identical function. The term tissue is also meant to encompass organs, which can be a functional grouping and organization of cells that can have different origins. The biological sample can be obtained from any tissue.

The biological samples can be obtained from different tissue samples from one or more humans or non-human animals. Suitable tissues can include connective tissues, muscle tissues, nervous tissues, epithelial tissues or a portion or combination thereof. Suitable tissues can also include all or a portion of a lung, a heart, a blood vessel (e.g., artery, vein, capillary), a salivary gland, a esophagus, a stomach, a liver, a gallbladder, a pancreas, a colon, a rectum, an anus, a hypothalamus, a pituitary gland, a pineal gland, a thyroid, a parathyroid, an adrenal gland, a kidney, a ureter, a bladder, a urethra, a lymph node, a tonsil, an adenoid, a thymus, a spleen, skin, muscle, a brain, a spinal cord, a nerve, an ovary, a fallopian tube, a uterus, vaginal tissue, a mammary gland, a testicle, a vas deferens, a seminal vesicle, a prostate, penile tissue, a pharynx, a larynx, a trachea, a bronchi, a diaphragm, bone marrow, a hair follicle, or a combination thereof. A biological sample from a human or non-human animal can also include a bodily fluid, secretion, or excretion; for example, a biological sample can be a sample of aqueous humour, vitreous humour, bile, blood, blood serum, breast milk, cerebrospinal fluid, endolymph, perilymph, female ejaculate, amniotic fluid, gastric juice, menses, mucus, peritoneal fluid, pleural fluid, saliva, sebum, semen, sweat, tears, vaginal secretion, vomit, urine, feces, or a combination thereof. The biological sample can be from healthy tissue, diseased tissue, tissue suspected of being diseased, or a combination thereof

In some embodiments, the biological sample is a fluid sample, for example a sample of blood, serum, sputum, urine, semen, or other biological fluid. In certain embodiments the sample is a blood sample. In some embodiments the biological sample is a tissue sample, such as a tissue sample taken to determine the presence or absence of disease in the tissue. In certain embodiments the sample is a sample of thyroid tissue.

The biological samples can be obtained from subjects in different stages of disease progression or different conditions. Different stages of disease progression or different conditions can include healthy, at the onset of primary symptom, at the onset of secondary symptom, at the onset of tertiary symptom, during the course of primary symptom, during the course of secondary symptom, during the course of tertiary symptom, at the end of the primary symptom, at the end of the secondary symptom, at the end of tertiary symptom, after the end of the primary symptom, after the end of the secondary symptom, after the end of the tertiary symptom, or a combination thereof. Different stages of disease progression can be a period of time after being diagnosed or suspected to have a disease; for example, at least about, or at least, 1, 2, 3, 4, 5, 6, 7, 8, 9, 10, 11, 12, 13, 14, 15, 16, 17, 18, 19, 20, 21, 22, 23 or 24 hours; 1, 2, 3, 4, 5, 6, 7, 8, 9, 10, 11, 12, 13, 14, 15, 16, 17, 18, 19, 20, 21, 22, 23, 24, 25, 26, 27 or 28 days; 1, 2, 3, 4, 5, 6, 7, 8, 9, 10, 11, 12, 13, 14, 15, 16, 17, 18, 19 or 20 weeks; 1, 2, 3, 4, 5, 6, 7, 8, 9, 10, 11 or 12 months; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 years after being diagnosed or suspected to have a disease. Different stages of disease progression or different conditions can include before, during or after an action or state; for example, treatment with drugs, treatment with a surgery, treatment with a procedure, performance of a standard of care procedure, resting, sleeping, eating, fasting, walking, running, performing a cognitive task, sexual activity, thinking, jumping, urinating, relaxing, being immobilized, being emotionally traumatized, being shock, and the like.

The methods of the present disclosure provide for analysis of a biological sample from a subject or a set of subjects. The subject(s) may be, e.g., any animal (e.g., a mammal), including but not limited to humans, non-human primates, rodents, dogs, cats, pigs, fish, and the like. The present methods and compositions can apply to biological samples from humans, as described herein.

A biological sample can be obtained by methods known in the art such as the biopsy methods provided herein, swabbing, scraping, phlebotomy, or any other suitable method. The biological sample can be obtained, stored, or transported using components of a kit of the present disclosure. In some cases, multiple biological samples, such as multiple thyroid samples, can be obtained for analysis, characterization, or diagnosis according to the methods of the present disclosure. In some cases, multiple biological samples, such as one or more samples from one tissue type (e.g., thyroid) and one or more samples from another tissue type (e.g., buccal) can be obtained for diagnosis or characterization by the methods of the present disclosure. In some cases, multiple samples, such as one or more samples from one tissue type (e.g., thyroid) and one or more samples from another tissue (e.g., buccal) can be obtained at the same or different times. In some cases, the samples obtained at different times are stored and/or analyzed by different methods. For example, a sample can be obtained and analyzed by cytological analysis (e.g., using routine staining). In some cases, a further sample can be obtained from a subject based on the results of a cytological analysis. The diagnosis of an immune disorder can include examination of a subject by a physician, nurse or other medical professional. The examination can be part of a routine examination, or the examination can be due to a specific complaint including, but not limited to, one of the following: pain, illness, anticipation of illness, presence of a suspicious lump or mass, a disease, or a condition. The subject may or may not be aware of the disease or condition. The medical professional can obtain a biological sample for testing. In some cases the medical professional can refer the subject to a testing center or laboratory for submission of the biological sample. The methods of obtaining provided herein include methods of biopsy including fine needle aspiration, core needle biopsy, vacuum assisted biopsy, incisional biopsy, excisional biopsy, punch biopsy, shave biopsy or skin biopsy. In some cases, the methods and compositions provided herein are applied to data only from biological samples obtained by FNA. In some cases, the methods and compositions provided herein are applied to data only from biological samples obtained by FNA or surgical biopsy. In some cases, the methods and compositions provided herein are applied to data only from biological samples obtained by surgical biopsy. A biological sample can be obtained by non-invasive methods, such methods including, but not limited to: scraping of the skin or cervix, swabbing of the cheek, saliva collection, urine collection, feces collection, collection of menses, tears, or semen. The biological sample can be obtained by an invasive procedure, such procedures including, but not limited to: biopsy, alveolar or pulmonary lavage, needle aspiration, or phlebotomy. The method of biopsy can further include incisional biopsy, excisional biopsy, punch biopsy, shave biopsy, or skin biopsy. The method of needle aspiration can further include fine needle aspiration, core needle biopsy, vacuum assisted biopsy, or large core biopsy. Multiple biological samples can be obtained by the methods herein to ensure a sufficient amount of biological material. Generic methods for obtaining biological samples are also known in the art and further described in for example Ramzy, Ibrahim Clinical Cytopathology and Aspiration Biopsy 2001 which is herein incorporated by reference in its entirety. The biological sample can be a fine needle aspirate of a thyroid nodule or a suspected thyroid tumor. The fine needle aspirate sampling procedure can be guided by the use of an ultrasound, X-ray, or other imaging device.

In some cases, the subject can be referred to a specialist such as an oncologist, surgeon, or endocrinologist for further diagnosis. The specialist can likewise obtain a biological sample for testing or refer the individual to a testing center or laboratory for submission of the biological sample. In any case, the biological sample can be obtained by a physician, nurse, or other medical professional such as a medical technician, endocrinologist, cytologist, phlebotomist, radiologist, or a pulmonologist. The medical professional can indicate the appropriate test or assay to perform on the sample, or the molecular profiling business of the present disclosure can consult on which assays or tests are most appropriately indicated. The molecular profiling business can bill the individual or medical or insurance provider thereof for consulting work, for sample acquisition and or storage, for materials, or for all products and services rendered.

A medical professional need not be involved in the initial diagnosis or sample acquisition. An individual can alternatively obtain a sample through the use of an over the counter kit. The kit can contain a means for obtaining said sample as described herein, a means for storing the sample for inspection, and instructions for proper use of the kit. In some cases, molecular profiling services are included in the price for purchase of the kit. In other cases, the molecular profiling services are billed separately.

A biological sample suitable for use by the molecular profiling business can be any material containing tissues, cells, nucleic acids, genes, gene fragments, expression products, gene expression products, and/or gene expression product fragments of an individual to be tested. Methods for determining sample suitability and/or adequacy are provided. The biological sample can include, but is not limited to, tissue, cells, and/or biological material from cells or derived from cells of an individual. The sample can be a heterogeneous or homogeneous population of cells or tissues. The biological sample can be obtained using any method known to the art that can provide a sample suitable for the analytical methods described herein.

Obtaining a biological sample can be aided by the use of a kit. A kit can be provided containing materials for obtaining, storing, and/or shipping biological samples. The kit can contain, for example, materials and/or instruments for the collection of the biological sample (e.g., sterile swabs, sterile cotton, disinfectant, needles, syringes, scalpels, anesthetic swabs, knives, curette blade, liquid nitrogen, etc.). The kit can contain, for example, materials and/or instruments for the storage and/or preservation of biological samples (e.g., containers; materials for temperature control such as ice, ice packs, cold packs, dry ice, liquid nitrogen; chemical preservatives or buffers such as formaldehyde, formalin, paraformaldehyde, glutaraldehyde, alcohols such as ethanol or methanol, acetone, acetic acid, HOPE fixative (Hepes-glutamic acid buffer-mediated organic solvent protection effect), heparin, saline, phosphate buffered saline, TAPS, bicine, Tris, tricine, TAPSO, HEPES, TES, MOPS, PIPES, cadodylate, SSC, MES, phosphate buffer; protease inhibitors such as aprotinin, bestatin, calpain inhibitor I and II, chymostatin, E-64, leupeptin, alpha-2-macroglobulin, pefabloc SC, pepstatin, phenylmethanesufonyl fluoride, trypsin inhibitors; DNAse inhibitors such as 2-mercaptoethanol, 2-nitro-5-thicyanobenzoic acid, calcium, EGTA, EDTA, sodium dodecyl sulfate, iodoacetate, etc.; RNAse inhibitors such as ribonuclease inhibitor protein; double-distilled water; DEPC (diethyprocarbonate) treated water, etc.). The kit can contain instructions for use. The kit can be provided as, or contain, a suitable container for shipping. The shipping container can be an insulated container. The shipping container can be self-addressed to a collection agent (e.g., laboratory, medical center, genetic testing company, etc.). The kit can be provided to a subject for home use or use by a medical professional. Alternatively, the kit can be provided directly to a medical professional.

One or more biological samples can be obtained from a given subject. In some cases, between about 1 and about 50 biological samples are obtained from the given subject; for example, about 1-50, 1-40, 1-30, 1-25, 1-20, 1-15, 1-10, 1-7, 1-5, 5-50, 5-40, 5-30, 5-25, 5-15, 5-10, 10-50, 10-40, 10-25, 10-20, 25-50, 25-40, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 biological samples can be obtained from the given subject. Multiple biological samples from the given subject can be obtained from the same source (e.g., the same tissue), e.g., multiple blood samples, or multiple tissue samples, or from multiple sources (e.g., multiple tissues). Multiple biological samples from the given subject can be obtained at the same time or at different times. Multiple biological samples from the given subject can be obtained at the same condition or different condition. Multiple biological samples from the given subject can be obtained at the same disease progression or different disease progression of the subject. If multiple biological samples are collected from the same source (e.g., the same tissue) from the particular subject, the samples can be combined into a single sample. Combining samples in this way can ensure that enough material is obtained for testing and/or analysis.

### Methods of Administering

Pharmaceutical formulations described herein can be administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the pharmaceutical compositions described herein are administered orally. In some embodiments, the pharmaceutical compositions described herein are administered topically. In such embodiments, the pharmaceutical compositions described herein are formulated into a variety of topically administrable compositions, such as solutions, suspensions, lotions, gels, pastes, shampoos, scrubs, rubs, smears, medicated sticks, medicated bandages, balms, creams or ointments. In some embodiments, the pharmaceutical compositions described herein are administered topically to the skin. In some embodiments, the pharmaceutical compositions described herein are administered by inhalation. In some embodiments, the pharmaceutical compositions described herein are formulated for intranasal administration. Such formulations include nasal sprays, nasal mists, and the like. In some embodiments, the pharmaceutical compositions described herein are formulated as eye drops. In some embodiments, the pharmaceutical compositions described herein are: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal. In some embodiments, the pharmaceutical compositions described herein are administered orally to the mammal. In certain embodiments, a composition described herein is administered in a local rather than systemic manner. In some embodiments, a composition described herein is administered with intraperitoneal injection. In some embodiments, a composition described herein is administered topically. In some embodiments, a composition described herein is administered systemically.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Injection can be conducted using sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, NJ) or phosphate buffered saline (PBS). In all cases, the composition must be sterile and should be fluid to the extent that easy syringability exists. It must be stable under the conditions of manufacture and storage and must be preserved against contamination from microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition.

Disclosed herein are compositions and methods for treatment of cancer related to tumor- intrinsic PRMT5 activity. Not intended to be bound by any theories, PRMT5 antagonizes the immune response by regulating antigen presentation/processing and production of IFN and chemokines. In embodiments provided herein, PRMT5-catalyzes methylation of IFI16/IFI204 represses activation of the intracellular DNA-induced cGAS/STING pathway and inhibits TBK1/IRF3 signaling and IFN and chemokine production. In embodiments further provided herein, one of the two putative methylation sites on IFI16/204 (R12) impactsSTING/cGAS signaling, as reflected by enhanced TBK1/IRF3 activation and concomitant IFN and chemokine production. Unexpectedly, data and studies provided herein also revealed the importance of IFI204 methylation in IFN and chemokine activation by dsRNA stimuli, consistent with an earlier report of the role of IFI16 in dsRNA-induced signaling . In additional embodiments, provided herein are compositions and methods of using genetic or pharmacological inhibition of PRMT5 in combination with checkpoint inhibitors, e.g. anti-PD1-therapy. In additional embodiments, the combined therapy of PRMT5 inhibition and anti-PD-1 treatment demonstrates enhanced efficiency of anti-PD-1 therapy on “cold” non-responsive tumors.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. General Methods

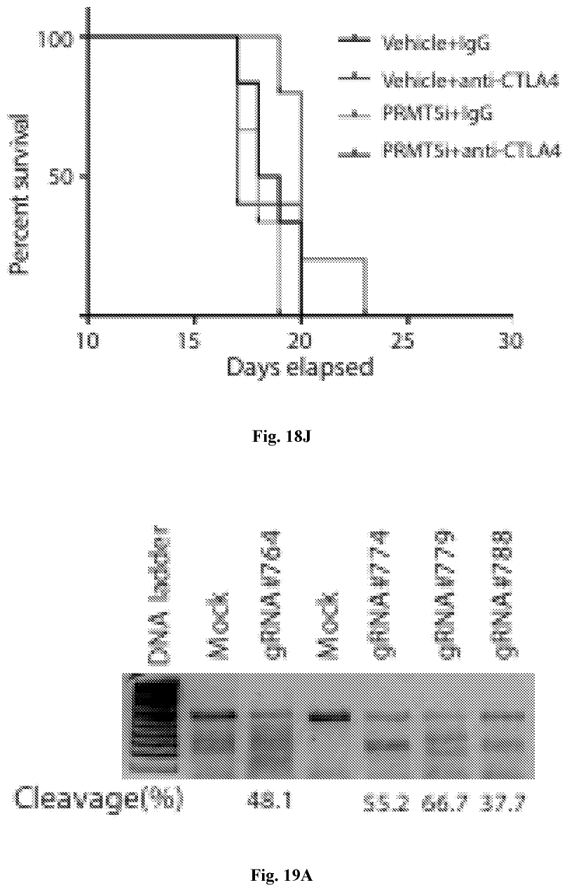

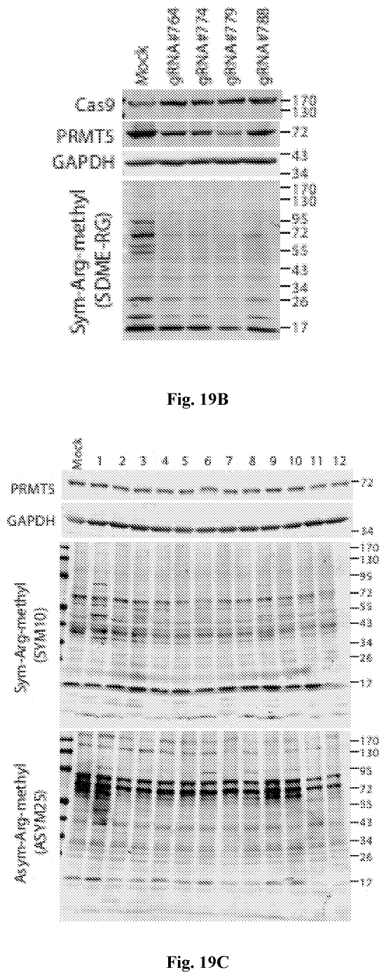

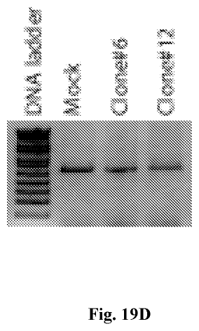

The objectives of the present study were to: (i) determine the effect of PRMT5 on control of the anti-tumor immune response, (ii) define relevant underlying molecular mechanisms, and (iii) evaluate therapeutic efficacy of PRMT5 inhibition alone or in combination with immune therapy in vivo. The present study relied on analyses of human melanoma databases, in vitro analyses of signal transduction and gene expression pathways for the type I IFN proinflammatory response and antigen processing/presentation, and in vivo animal studies monitoring tumor growth and response to therapies.PRMT5 immune suppressive function in syngeneic murine models of melanoma was evaluated, using less-immunogenic B16 and YUMM1.7 cells for loss of function studies and immunogenic YUMMER1.7 cells for gain of function studies. Genetic inactivation of PRMT5 was restricted to use of lentiviral shRNAs (multiple) in studies of both cultured cells and in vivo, since total ablation of PRMT5 using CRISPR/CAS9 approaches results in complete lethality (FIG. 19). The studies were thus complemented using a first-in-class pharmacological inhibitor for PRMT5, EPZ015666, which provided independent confirmation to genetic-based inhibition studies. Phenotypes seen in melanoma cells subjected to PRMT5 knockdown were confirmed using pharmacological PRMT5 inhibitors as well as through analysis of PRMT gain of function in overexpression assays. Animal care and related procedures followed institutional guidelines and was conducted with approval of the Institutional Animal Care and Use Committee of Sanford Burnham Prebys Medical Discovery Institute. Animal cohort sizes were designed to detect differences in treatment effects at 80% power (alpha error rate =0.05), with the exception of studies conducted to assess immune phenotypes. Mice with unexpected and severe skin atopic dermatitis were excluded. All experiments were conducted 2-3 times, except for tumor studies in which specific immune cells were depleted; in those analyses cohort size was sufficient to support the statistical power stated above. Each experiment consisted of 3-4 technical replicates. Sample identity for tumor studies were blinded to the investigator who grafted them into mice.

Cell Culture and Treatment.

Human and murine melanoma cells [B16F10, purchased from ATCC; YUMM1.7 and YUMMER1.7, obtained from Yale University; A375 and WM115, obtained from the Wistar Institute ]; and HEK293T cells, from ATCC] were maintained in DMEM (Hyclone) containing 10% fetal bovine serum (Omega Scientific and PEAK serum) plus penicillin/streptomycin (10000 U/ml, Thermo scientific) in 5% CO2 at 37° C. Stably-transduced cells were maintained withappropriate antibiotics, including puromycin (InvivoGen, 1 μg/ml) and blasticidin (InvivoGen, 10μg/ml). Cells were maintained in growth phase and did not exceed 80% confluency. Cells were stimulated by treatment with (i) interferon gamma (R&D Systems), (ii) by transfection with LMW (low molecular weight)/HMW (high molecular weight) poly(I:C) (InvivoGen, 250 ng/ml) or by (iii) transfection of vaccinia virus dsDNA V70mer (500 ng/ml for detecting Ifnb1/chemokine expression and 1.5 μg/ml for detecting TBK1/IRF3 activation). V70mer was prepared by annealingthecomplementingoligonucleotides

5′-CCATCAGAAAGAGGTTTAATATTTTTGTGAGACCATCGGGGCCGCGCC
TCCCCCGCG449AGGCCGCCGGCG-3′.

Animal experiments. All animal experiments were conducted with approval of the Institutional Animal Care and Use Committee of Sanford Burnham Prebys Medical Discovery Institute (AUF#18-044). The murine melanoma lines B16F10, YUMM1.7 , and YUMMER1.7 were injected subcutaneously (2.0×10\hat{ }5 cells of B16F10 or YUMM1.7; 4.0 ×10\hat{ }5 cells of YUMMER1.7) into the lower right flank of 6-8-week-old male C57BL/6 (B16F10, YUMM1.7, YUMMER1.7) or Nod-Scid-Gamma (NSG) (B16F10, YUMMER1.7) mice. To induce transduced inducible shPRMT5, doxycycline (10 mg/ml, Fisher Bioreagents) was prepared in methylcellulose solution (0.5% hydroxylmethylcellulose, 0.2% Tween80) and administered to mice (0.2 ml, oral gavage, QD)Tumor sizes were monitored using calipers. At indicated time points, tumors were collected, 21 weighed and assessed for immune phenotypes using flow cytometry or immunofluorescence. To assess efficacy of immune checkpoint antibodies, mice were grafted with B16F10 or YUMM1.7 (2.0×10\hat{ }5 cells, s.c.) cells and treated with 200 μg control IgG [rat IgG2a; BE0089 (BioXcell)], anti-CD152 (CTLA-4) [9H10 (BE0131, BioXcell)] or anti-CD279 (PD-1) [RMP1-14 (BE0146, BioXcell)]. Antibodies were injected (i.p.) 3 - 5 times (every 3 days starting from the indicated date). The PRMT5 inhibitor GSK3326595 (Chemitek) was prepared in methylcellulose solution and administered to mice (40 mg/kg, oral gavage, QD). To deplete NK or CD8+ cells, mice were treated with anti-NK1.1 antibody [PK136 (BE0036, BioXcell)] or anti-CD8 antibody [2.43(BE0061, BioXcell), respectively; controls were treated with 200 μtg IgG [rat IgG2b (BE0090, BioXcell)]. Antibodies were injected (i.p.) every 3 days starting one day prior to tumor cell inoculation. The efficiency of depletion was assessed using flow cytometry of blood samples collected at day 8 after tumor inoculation. To assess percent survival of animals, mice bearing tumors exceeding 2,000 mm³ were defined as “dead”. Gene set enrichment (GSEA) and Ingenuity Pathway (IPA) analyses

GSEA was performed using a GSEA Desktop Application downloaded from software.broadinstitute.org/gsea/. Gene expression (RNA-seq) data from specimens from human melanoma patients obtained from the TCGA (The Cancer Genome Atlas) or GEO (Gene Expression Omnibus) databases was used to identify genes differentially-expressed between patient groups with characteristics of interest (low/high expression of PRMT5 or MTAP). Curated sets of hallmark (50 gene sets) and C5 GO (5917 gene sets) genes from the Molecular Signature Database (MSigDB v6.1) served as input. High-ranked gene sets from the analysis were presented along with the enrichment plot, NES (normalized enrichment score), nominal p value FDR-q value and a heatmap of the corresponding gene set. Comparison of multiple gene sets was summarized with a heatmap drawn with NES and FDR-q values. For IPA (Qiagen Inc), differentially expressed genes with an unpaired t-test p value of <0.05 and a fold-difference of >2.0 between low and high groups were analyzed using core analysis. High-ranked canonical pathways were presented along with p values (right-tailed Fisher's exact test), ratio (coverage of pathway), and Z score with pathway directionality (filled blue bars).

Statistical Analysis.

Statistical analyses were performed using Prism software (version 7.00, GraphPad). For comparison of means of two groups with normal (or approximately normal) distributions, an un-paired t-test was applied. To compare means between >2 groups, one-way analysis of variance (ANOVA) was used with multiple comparison corrections (Dunnett's or Tukey's test). For animal experiments, two-way ANOVA (time and treatment) was used with Tukey's multiple comparison test. For Kaplan-Meier plots to compare overall survival, a log-rank test was used to determine significance of differences between groups. To evaluate response to therapy in a mouse model, Fisher's exact test (version 7.00, GraphPad) was used. For that analysis, a tumor with volume <50% of control tumors was defined as “responding to treatment”. For all analyses, a difference with p<0.05 was considered significant, unless specified.

DNA Constructs, Mutagenesis and Transfection.

DNA plasmids were constructed using the pLX304 Gateway system (Addgene, #25890). Briefly, PCR-amplified cDNAs for mouse Sharpin, Ifi204, Wdr77 or Nlrc5 were cloned into the pLX304 lentiviral gateway vector using LR clonase II and a pENTR-D-TOPO cloning kit (Thermo Fisher Scientific). Mouse Prmt5 was cloned into the EcoRI/BamHI sites of pLenti-puro (Addgene #39481). For mouse Nlrc5, 2 PCR products (N¬and C-terminal fragments) were separately cloned into pENTR-D-TOPO using ligation of the Hpal fragments. Human PRMT5 and SHARPIN DNA constructs were established as described (1), and pLX304-IFI16 was obtained from DNASU (Arizona State University Biodesign Institute). pENTR-D-TOPO-IFI204 R12A, 538A and RR12/538AA mutant plasmids were generated using the QuikChange II XL Site Directed Mutagenesis Kit (Agilent) and the inserts were subsequently cloned into pLX304 (lentiviral) expression plasmids. Gene-specific shRNA lentiviral vectors with a pLK0.1 backbone were obtained from the La Jolla Institute for Immunology RNAi CenterProduction and Infection of Viral Particles.

Lentiviral particles were prepared using standard protocols. Briefly, HEK293T cells were transfected with lentiviral plasmid and the second-generation packaging plasmids delta R8.2 and Vsv-G (Addgene) using Calfectin (SignaGen). Viral supernatants were collected 48 h later, filtered using a syringe filter (0.45 [M pore size) and combined with polybrene (8 μg/ml, Sigma) to infect melanoma lines. To establish stably-transduced cells, efficiently-infected cells were selected in either puromycin (InvivoGen, 1˜2μg/ml) or blasticidin (InvivoGen, 10 μg/ml), as appropriateImmunoblotting and Immunoprecipitation.

Immunoblotting was performed using standard protocols. Melanoma cells were lysed by incubation in RIPA buffer [50 mM Tris-HC1, pH7.4, 1% (v/v) NP40, 0.1% (w/v) sodium deoxycholate, 0.1% (w/v) sodium dodecyl sulfate, 150 mM NaCl, 1mM EDTA, and protease and phosphatase inhibitor cocktails (Thermo Fisher Scientific)] and freeze-thawed 3 times. For immunoprecipitation HEK293T cells were lysed in 1% Triton X-100 buffer [50 mM Tris-HC1, pH7.4, 1% (v/v) Triton X-100, 150 mM NaCl, 1 mM EDTA, and protease and phosphatase inhibitor cocktails (Thermo Fisher Scientific)] and freeze-thawed 3 times. Lysates were then incubated overnight with indicated antibodies and then for 4 hr with protein A/G agarose beads (Santa Cruz Biotechnology) under gentle mixing. Proteins were eluted by addition of lysis buffer and boiled in Laemmli buffer before separation on SDS-PAGE and transfer to a PVDF membrane. Membranes were incubated for 1 h at room temperature with blocking solution [TBS (Tris-buffered Saline); 10 mM Tris-HC1, pH8.0, 150 mM NaCl)] containing 0.1% Tween 20 and 5% nonfat milk followed by incubation overnight at 4° C. with appropriate primary antibodies. Membranes were washed with TBS and incubated 1 h at room temperature with secondary antibody [Alexa 680-conjugated goat anti-rabbit, goat anti-mouse, donkey anti-goat (Life Technologies) or IRDye 800-conjugated goat anti-mouse (Rockland Immunochemicals)], or HRP-conjugated anti-mouse or anti-rabbit IgG antibodies. TidyBlot anti-rabbit secondary antibody (Bio-Rad) was used to detect immunoprecipitated proteins (SHARPIN). Blots were treated with HRP-conjugated antibodies and SuperSignal West Pico or Femto chemiluminescent substrate (ThermoFisher scientific). Protein bands were visualized and quantified using an Odyssey Infrared Imaging System (LiCor Biosciences) or ChemiDoc Imaging System (Bio-Rad). The following antibodies were used to detect proteins or tags: PRMT5 (A-11, Santa Cruz Biotechnology), WDR77 (FG-4, Santa Cruz Biotechnology), IFI16 (1G7 and G-4, Santa Cruz Biotechnology), NLRC5 (B-10, Santa Cruz Biotechnology), LMP2/PSMB9 (EPR13785, Abcam), GAPDH (C65, Santa Cruz Biotechnology), MYC (9E10, Santa Cruz Biotechnology), SHARPIN (AFP128, EMD Millipore), Dimethyl-Arginine, Symmetric (SYM10, EMD Millipore), Symmetric Di-Methyl Arginine Motif (#13222, Cell Signaling), TAP1 (#12341, Cell Signaling), STING (D2P2F or D1V5L, Cell Signaling), phospho-STING (Ser365) (D1C4T, Cell Signaling), phospho-TBK1(D52C2, Cell Signaling), TBK1 (#3013, Cell Signaling), phospho-IRF3 (4D4G, or D601M,Cell Signaling), IRF3 (D83B9, Cell Signaling), IFI204 (Novus), V5 (A190, Bethyl Laboratories or 7/4, Biolegend), HIS-tag (J099B12, Biolegend) and FLAG (M2, Sigma). Semi-native PAGE was performed as described (2) . Briefly, cell lysates prepared from RIPA buffer were boiled in Laemmli buffer without □-mercaptoethanol. Samples were separated in SDS-PAGE (4-20%) and transferred to PVDF membrane. STING polymer was analyzed using BlueNative(BN) PAGE (3). As described, cells were solublized by rotating for 30 min at 4° C. using native-lysis buffer [20 mM HEPES (pH7.0), 25 mM NaCl, 10% glycerol, 1% DDM, protease inhibitor cocktail]. Lysates were run on a BN-PAGE (4-15%) Gel (4) and transferred to a PVDF membrane. STING dimers and polymers were detected using a STING antibody (D2P2F, Cell Signaling),Immunofluorescence

Paraffin tumor sections were prepared for immunofluorescence (IF) using rehydration and antigen retrieval processes based on the IHC protocol. Sections were then stained with primary antibodies CD4 (Abcam, 1:200 dilution), and CD8 (eBioscience, 4SM15, 1:100 dilution) and then with corresponding secondary antibodies conjugated with Alexa488 (ThermoFisher Scientific, 1:500 dilution). The same slides were counterstained with DAPI (Vector Laboratori). IF-stained slides were visualized using a fluorescence microscope aided by Slidebook software and further assessed following scanning using the Aperio system. Ratio of area was calculated by dividing the area containing CD4+ or CD8+ with that of DAPI+ (Image J).

RNA Extraction and Quantitative PCR (qPCR).

Total RNA was isolated from cells with GenElute (Sigma-Aldrich) and reverse transcribed using high capacity cDNA synthesis kits (Applied Biosystems). qPCR was performed with a CFX Connect Real-Time PCR Detection System (Bio-Rad) using FastStart Universal SYBR Green Master Mix (Life Technologies). Primer sequences were as follows:

H3.3
(used for an internal control for nomlaization)
5′- TGTGGCCCTCCGTGAAATC-3′,
5′-GGCATAATTGTTACACGTTTGGC-3′,
mouse CXCL10
5′-CCAAGTGCTGCCGTCATTTTC-3′,
5′-GGCTCGCAGGGATGATTTCAA-3′,
mouse CCL5
5′- CTCACCATATGGCTCGGACA-3′,
5′-CTTCTCTGGGTTGGCACACA-3′,
mouse IFNB1
5′- CAGCTCCAAGAAAGGACGAAC-3′,
5′-GGCAGTGTAACTCTTCTGCAT-3′,
mouse NLRC5
5′-GTGCCAAACGTCCTTTTCAGA-3′,
5′-AGTGAGGAGTAAGCCATGCTC-3′,
mouse TAP1
5′- GGACTTGCCTTGTTCCGAGAG-3′,
5′-GCTGCCACATAACTGATAGCGA-3′,
mouse B2M
5′- TTCTGGTGCTTGTCTCACTGA-3′,
5′-CAGTATGTTCGGCTTCCCATTC-3′,
mouse PSMB9
5′-CATGAACCGAGATGGCTCTAGT-3′,
5′-TCATCGTAGAATTTTGGCAGCTC-3′,
IFI204
5′- GAGCAAGGCGGCTAAGGAA-3′,
5′-GCTGTGGAGTATTGGTGACTG-3′,
mouse PRMT5
5′- CTGAATTGCGTCCCCGAAATA-3′,
5′-AGGTTCCTGAATGAACTCCCT-3′,
mouse WDR77
5′-CTTGCTGTGCTGGATTCAAGC-3′,
5′-CAACTGTGGTAAGAAGGGAGTG-3′,
mouse Pd-11(Cd274)
5′-TGGGGCCTAAGCCTATGTCT-3′,
5′-CTCCCAAGGGTGGCTTTAGG-3′,
mouse Pd-l2(Pdcd1lg2)
5′-CTGCCGATACTGAACCTGAGC-3′,
5′-GCGGTCAAAATCGCACTCC-3′.
Gene silencing.

To KD Prmt5, respective pLKO.1 clones were purchased (La Jolla institute for immunology). Two shRNAs, shPRMT5-1 (TRCN0000181891) and shPRMT5-1 (TRCN00001182569), were used to establish stable cultures. For inducible knockdown, oligonucleotides of the same sequence were cloned into pLKO.1-Tet ON (Addgene #21915). To silence STING, cells were transfected with MISSION esiRNA (Millipore Sigma) using JetPrime transfection agent.

Mass Spectrometry

To identify SHARPIN-interacting proteins, WM115 cells were transfected with Flag-tagged SHARPIN or control empty plasmid. Cell lysates were prepared using 1% Triton X-100 buffer [50 mM Tris-HCl, pH7.4, 1% (v/v) Triton X-100, 150 mM NaCl, 1 mM EDTA, and protease and phosphatase inhibitor cocktails (Thermo Fisher Scientific)]. Lysates were pre-cleared with protein A/G beads (Santa Cruz Biotechnology) for 1 h at 4° C. followed by immunoprecipitation using FLAG-M2-agarose beads (Sigma-Aldrich) overnight at 4° C. Beads were then washed with lysis buffer and TBS containing lx protease inhibitor cocktail before being subjected to on-bead tryptic digestion followed by mass spectrometry. In Vitro Methylation Assay.

Wildtype or mutant (R12A, R538A, or RR12/538AA) forms of IFI204 were expressed and immunopurified from HEK293T cells transfected with corresponding plasmids (V5-tagged protein purification kit, MLB international). Purified IFI204 (200 ng) and histone 4 (1-2 μg) proteins were incubated with 500 ng PRMT5/MEP50,2 μCi of S-adenosyl-L-methyl-3H-methionine (3H-SAM, PerkinElmer) and/or PRMT5i (GSK3326595, 10 μM) in a 30-50 μreaction mixture with HMTase (histone methyltransferase) buffer (25 mM NaCl, 25 mM Tris, pH 8.8) for 120 min at 30° C. Reaction products were resolved on 4-20% gradient SDS-PAGE gels and transferred to PVDF membranes. Membranes were treated with enhancing spray (PerkinElmer), air-dried, and autoradiographed.

Assessment of cell growth in culture.

An ATPlite luminescence assay system (Perkin Elmer) was used to measure cell growth. Briefly, 2,500-5,000 cells were placed into 96-well plates with clear bottoms (Nunc). After 3-5 days, ATPlite working solution was added and luminescence measured using a Flexstation 3 microplate reader (Molecular Devices). Doxycycline (1 μg/ml, Fisher Bioreagent) was used to induce shPRMT5 in YUMM1.7 cells).

For immune phenotyping, B16F10 tumors were collected and processed by chopping and mincing using a tube-secured cell strainer (70 p.m poresize, Falcon) to prepare single cells for flow cytometry. YUMMER1.7 cells were processed by chopping, following incubation in Collargenase D solution [0.1% (w/v) Collagenase D, 0.5% (w/v) BSA, 100 μg/ml DNase in PBS] for 1 hr at 30° C. and mincing using cells strainer. Total cells were counted and a fraction (2 x 106) of cells in FACS staining buffer (phosphate-buffered saline, pH7.4, containing 1 FBS) were treated with the following sets of antibodies (1:200 dilution): cocktail 1 [CD45.2 (AF700), CD8 (PB), CD4 (BV605), CD44 (APCCy7), CD25 (FITC), FoxP3 (APC) and purified CD16/32] and cocktail 2 [ CD45.2 (AF700), MHCII (PB), CD11C (APC), CD11b (APCCy7), GR1 (PE), F4/80 (FITC), NK1.1 (BV605) and purified CD16/32] for 20 min at 4° C. All antibodies were from Biolegend. Stained cells were fixed in 1% formaldehyde (Sigma) in PBS (pH7.4) for 15 min at 4° C. and analyzed with BD LSRFortessa (BD Biosciences) flow cytometry. For intranuclear staining of FoxP3, cells were fixed and permearbilized using a FoxP3/Transcription factor Fixation/Permearbilization kit (ThermoFisher Scientific). For the assessment of intracellular cytokine in infiltrated CD4+ and CD8+ cells, a fraction (2×106) of cells prepared from tumors were stimulated with PMA (10 ng/ml)/Ionomycin (0.5 μg/ml)/BFA (1 μg/ml) for 16hr. Cells were stained with antibody cocktail for surface markers [CD45.2, CD4, and CD8] and followed by staining with intraceullar cytokine antibodies [IFN gamma (APC), TNF alpha(FITC) and IL-2(PE)]. The number of specific immune cells per gram of tumor was calculated based on the percentage of specified immune cells identified in FACS (relative to the total number of tumors cells) per tumor weight. To assess surface MHCI expression, B16F10 cells (1 x 106) in FACS buffer were stained with isotype control, anti-H2Kb (FITC), or anti-interferon gamma receptor beta chain (PE) antibodies. Cells were fixed and analyzed as above. To assess effects efficiency of in vivo depletion of CD8 T or NK cells, immune cells were isolated from mouse blood and RBCs were lysed using hypotonic buffer [15 mM NH4C1, 10 mM KHCO3, 0.1mM Na2EDTA] for 1 hr at 4° C. Immune cells were then stained and analyzed as described above.

### Example 2. PR1VIT5 Expression is Inversely Correlated with an Antitumor Immune Signature

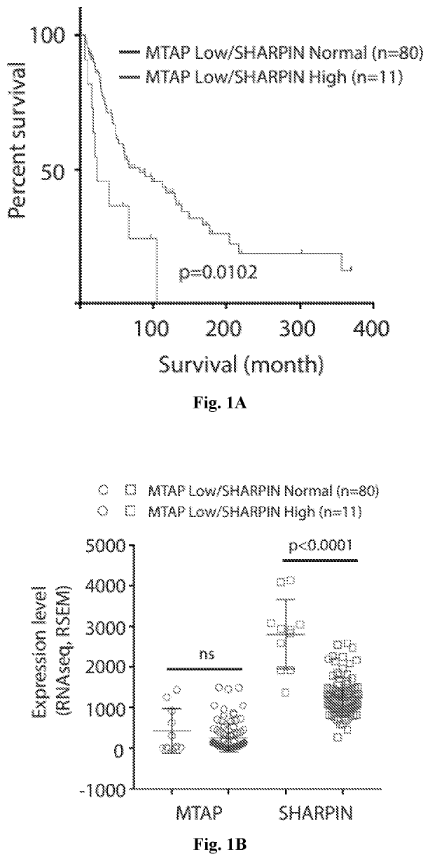

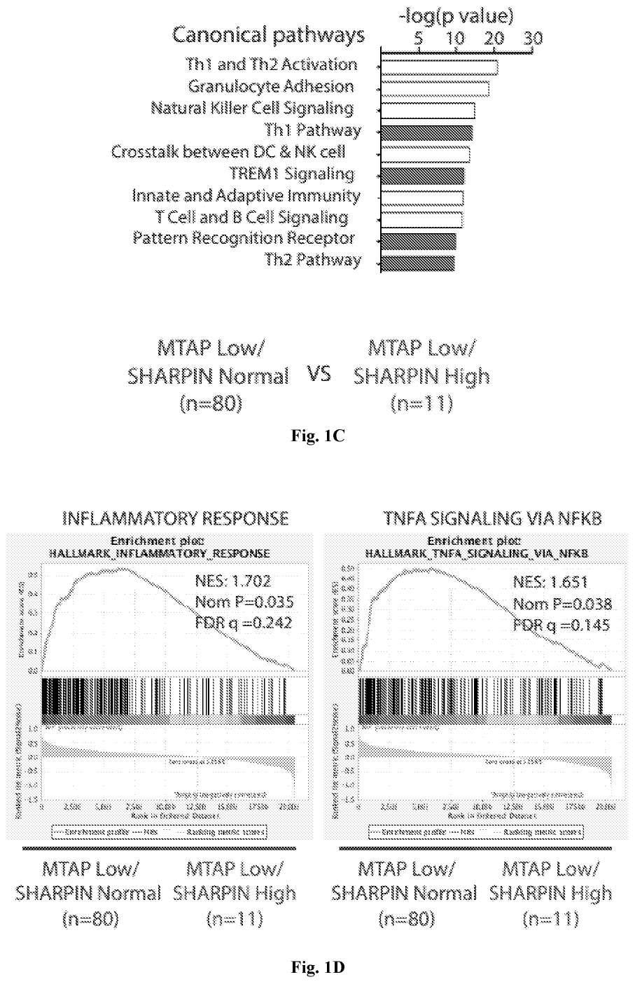

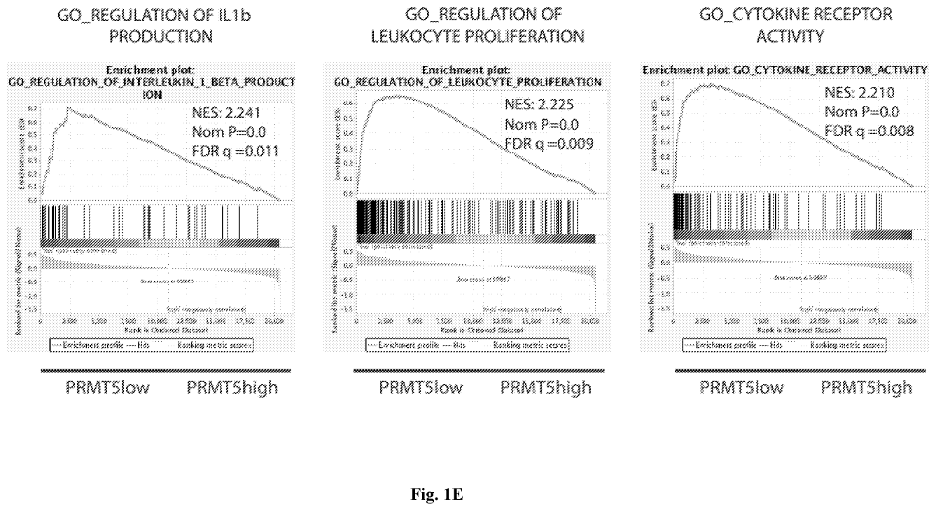

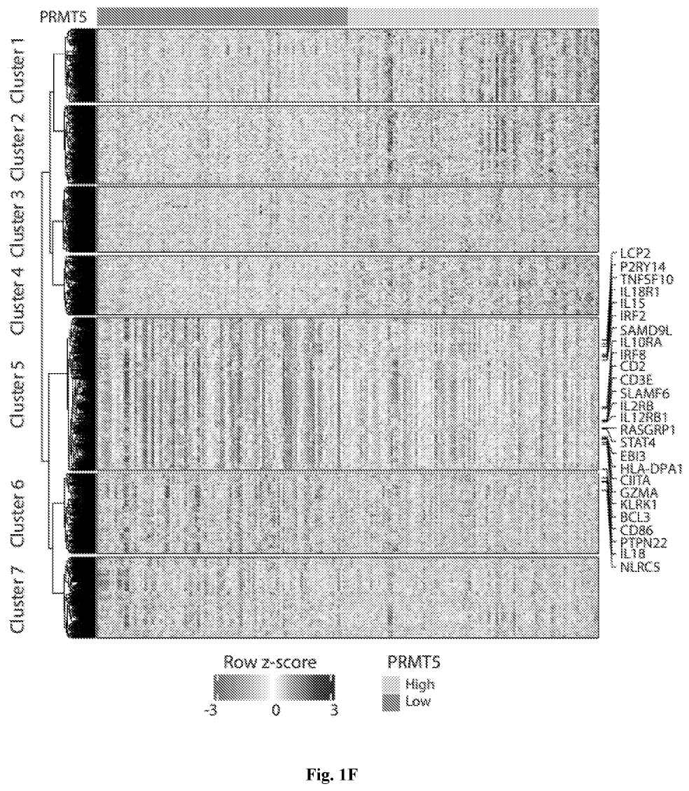

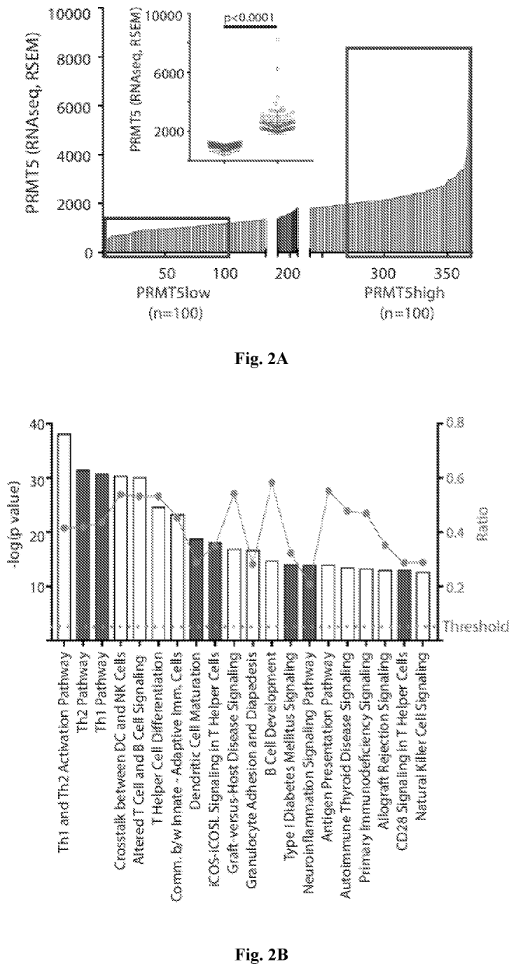

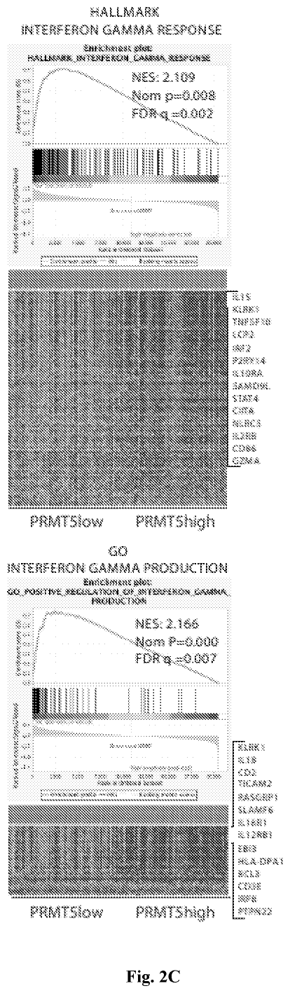

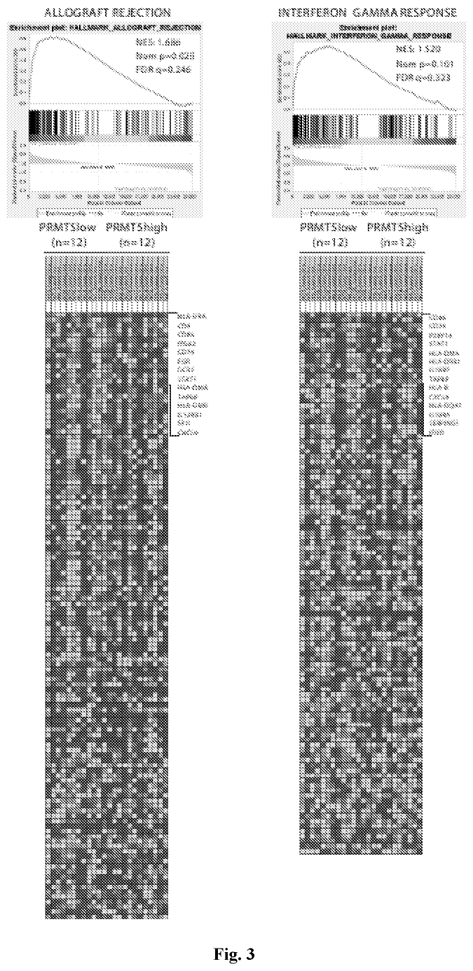

PRMT5 interaction with SHARPIN (SHANK-associated RH domain-interacting protein),was recently identified, which augments its methytransferase activity (Tamiya et al., The Journal of clinical investigation 2018;128(1):517-30, incorporated herein in its entirety). SHARPIN expression was assessed in cohorts of melanoma tumor specimens. Following analysis of melanoma patient datasets, low SHARPIN expression in MTAP-deleted tumor was shown to be associated with better survival (FIGS. 1A and 1B). To identify regulatory pathways that may be modulated by SHARPIN, differentially-expressed genes (DEGs) were evaluated in cohorts of metastatic melanoma patients. MTAP deleted tumors harboring low SHARPIN expression exhibited enrichment of genes associated with immune related pathways (Th1/Th2, IL-2/Stat5, TNFalpha) relative to MTAP-deleted specimens with high SHARPIN expression (FIGS. 1C and 1D, and Table 1) suggesting a function of SHARPIN in controlling immune phenotypes in MTAP-deleted melanoma. To determine whether PRMT5 expression levels in melanoma tumors were associated with expression of a particular gene set, DEGs in melanoma cohorts (TCGA) showing low versus high PRMT5 expression were analyzed using IPA and GSEA (FIG. 2A). low PRMT5 melanomas exhibited enriched expression of immune-associated genes (Th1 and Th2 activation pathways, allograft rejection, inflammatory response, and interferon gamma response/production) (FIGS. 1E, 1F, 2B and 2C; Tables 2 and 3), similar to changes seen in low-SHARPIN, MTAP-deleted melanomas. Analysis of an independent cohort of melanomas (GSE78220, n=27) also identified enrichment of an immune gene signature associated with allograft rejection and the interferon gamma response in PRMT5 -low tumors (FIG. 3).

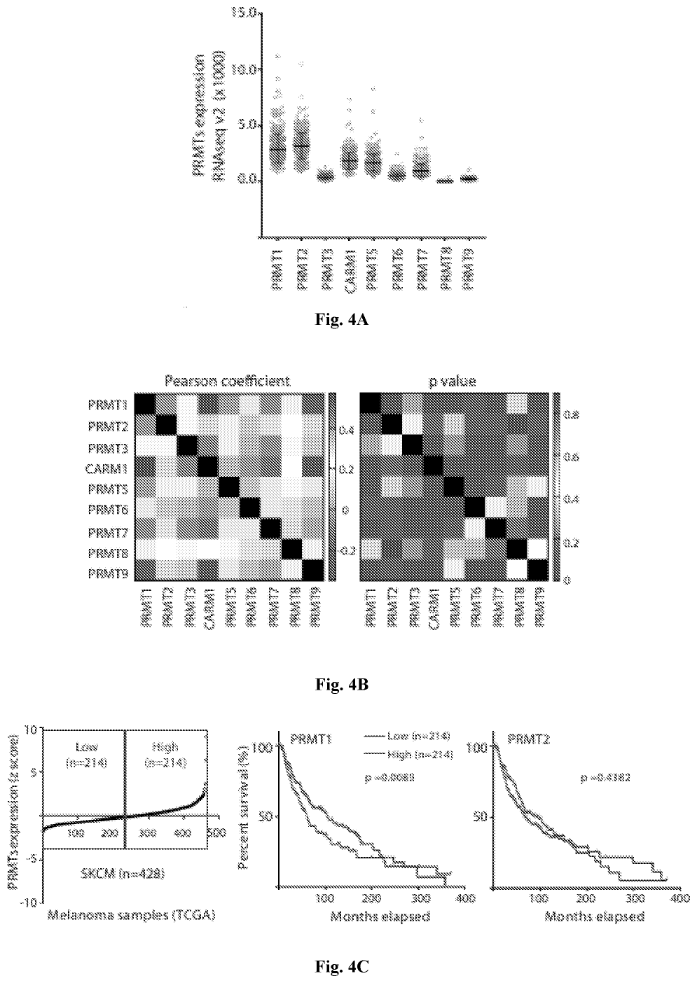

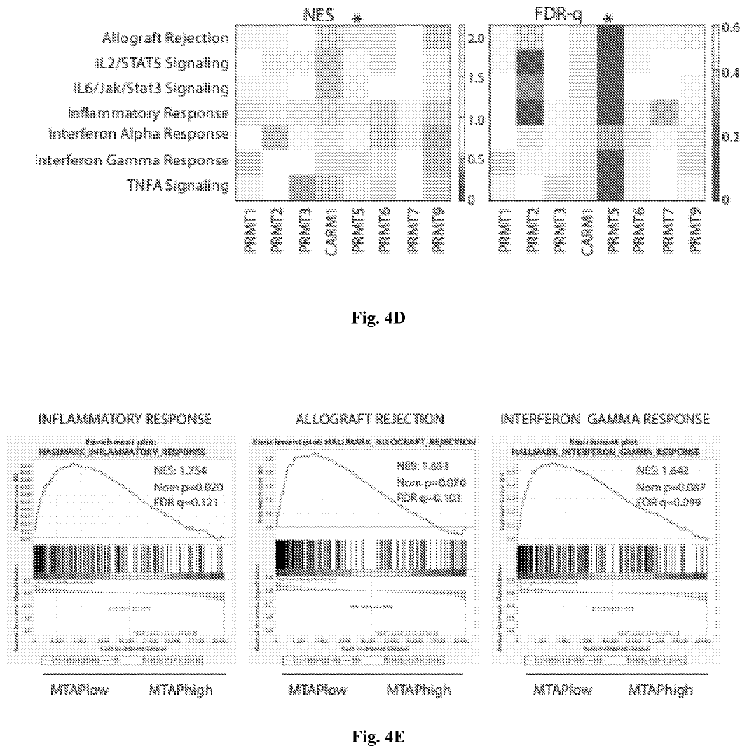

Among the PRMT family members, PRMT5 , PRMT1, PRMT2, CARM1 and PRMT7 were observed at relatively high levels in human melanoma specimens (FIG. 4A), of which, PRMT5, PRMT1 and CARM are co-expressed in those melanoma specimens (FIG. 4B). The high PRMT5, PRMT1 and CARM1 expression that were observed in melanoma specimens coincided with lower survival (FIG. 4C). PRMT5 expression exhibited strongest inverse correlation with expression of immune response genes (FIG. 4D). Correspondingly, melanomas harboring low-MTAP expression and low PRMT5 activity exhibited enrichment of an immune pathway signature (FIG. 4E), supporting a role for PRMT5 in tumor immunity.

TABLE 1
Top-enriched hallmark gene sets from GSEA
of DEGs from SKCM METASTATIC (TCGA) melanoma
patients with MTAPlow/SHARPINlow versus
MTAPlow/SHARPIN high expression

| | | NOM | FDR |
|---|---|---|---|
| NAME | NES | p-val | q-val |
| KRAS SIGNALING UP | 1.749 | 0.004 | 0.343 |
| IL2/STAT5 SIGNALING | 1.681 | 0.022 | 0.193 |
| COMPLEMENT | 1.658 | 0.032 | 0.176 |
| INFLAMMATORY RESPONSE | 1.702 | 0.035 | 0.242 |
| TNFA SIGNALING VIA NFKB | 1.651 | 0.038 | 0.145 |
| IL6/JAK/STAT3 SIGNALING | 1.628 | 0.05 | 0.145 |
| ANDROGEN RESPONSE | 1.458 | 0.052 | 0.249 |
| TGF BETA SIGNALING | 1.491 | 0.056 | 0.251 |
| APOPTOSIS | 1.429 | 0.061 | 0.265 |
| PROTEIN SECRETION | 1.464 | 0.072 | 0.265 |
| UV RESPONSE DN | 1.389 | 0.09 | 0.297 |
| INTERFERON GAMMA RESPONSE | 1.508 | 0.126 | 0.263 |

TABLE 2
Top-enriched hallmark gene sets from GSEA of DEGs
from SKCM METASTATIC (TCGA) melanoma patients
with PRMT5 low versus PRMT5 high expression.

| | | NOM | FDR |
|---|---|---|---|
| NAME | NES | p-val | q-val |
| ALLOGRAFT REJECTION | 2.222 | 0 | 0 |
| INFLAMMATORY RESPONSE | 2.156 | 0 | 0.001 |
| IL6 JAK STAT3 SIGNALING | 2.13 | 0 | 0.002 |
| INTERFERON GAMMA RESPONSE | 2.109 | 0 | 0.002 |
| IL2/STAT5 SIGNALING | 2.092 | 0 | 0.001 |
| COMPLEMENT | 2.072 | 0 | 0.001 |
| KRAS SIGNALING UP | 2.011 | 0 | 0.004 |
| TNFA SIGNALING VIA NFKB | 1.932 | 0.004 | 0.01 |
| INTERFERON ALPHA RESPONSE | 1.733 | 0.05 | 0.051 |
| APOPTOSIS | 1.667 | 0.008 | 0.073 |

TABLE 3
Top-enriched GO gene sets from GSEA of DEGs from SKCM METASTATIC (TCGA)
melanoma patients with PRMT5 low versus PRMT5 high expression.

| | | NOM | FDR |
|---|---|---|---|
| NAME | NES | p-val | q-val |
| GO NEGATIVE REGULATION OF CELL ACTIVATION | 2.255 | 0.000 | 0.020 |
| GO REGULATION OF INTERLEUKIN 1 BETA PRODUCTION | 2.241 | 0.000 | 0.011 |
| GO REGULATION OF HOMOTYPIC CELL CELL ADHESION | 2.229 | 0.000 | 0.010 |
| GO REGULATION OF LEUKOCYTE PROLIFERATION | 2.225 | 0.000 | 0.009 |
| GO REGULATION OF CELL ACTIVATION | 2.215 | 0.000 | 0.009 |
| GO CYTOKINE RECEPTOR ACTIVITY | 2.210 | 0.000 | 0.008 |
| GO POSITIVE REGULATION OF LEUKOCYTE PROLIFERATION | 2.206 | 0.000 | 0.007 |
| GO REGULATION OF LEUKOCYTE MEDIATED CYTOTOXICITY | 2.206 | 0.000 | 0.007 |
| GO REGULATION OF T CELL PROLIFERATION | 2.204 | 0.000 | 0.006 |
| GO SIDE OF MEMBRANE | 2.197 | 0.000 | 0.006 |
| GO REGULATION OF CELL CELL ADHESION | 2.195 | 0.000 | 0.006 |
| GO POSITIVE REGULATION OF INTERLEUKIN 1 BETA PRODUCTION | 2.193 | 0.000 | 0.006 |
| GO REGULATION OF CELL KILLING | 2.192 | 0.000 | 0.006 |
| GO NEGATIVE REGULATION OF IMMUNE SYSTEM PROCESS | 2.180 | 0.000 | 0.006 |
| GO LEUKOCYTE ACTIVATION | 2.175 | 0.000 | 0.006 |
| GO POSITIVE REGULATION OF CELL CELL ADHESION | 2.172 | 0.000 | 0.006 |
| GO POSITIVE REGULATION OF INTERFERON GAMMA PRODUCTION | 2.166 | 0.000 | 0.007 |
| GO POSITIVE REGULATION OF CELL ACTIVATION | 2.163 | 0.000 | 0.007 |
| GO REGULATION OF CYTOKINE SECRETION | 2.162 | 0.000 | 0.007 |

### Example 3. PRMT5 Inhibition Attenuates Tumor Growth in an Immunocompetent Murine Melanoma Model

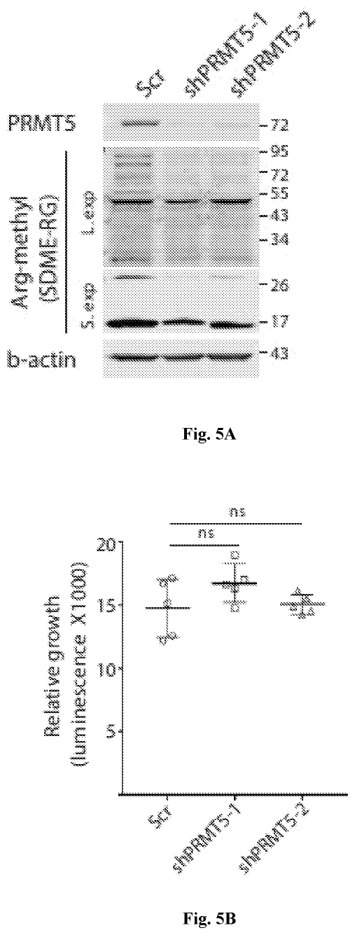

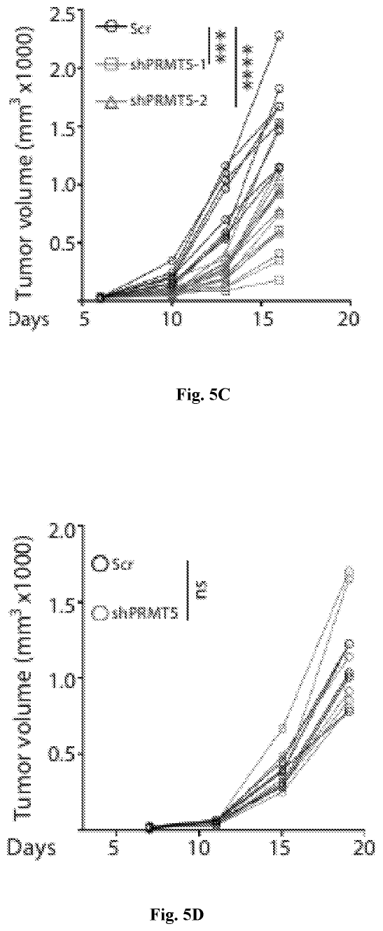

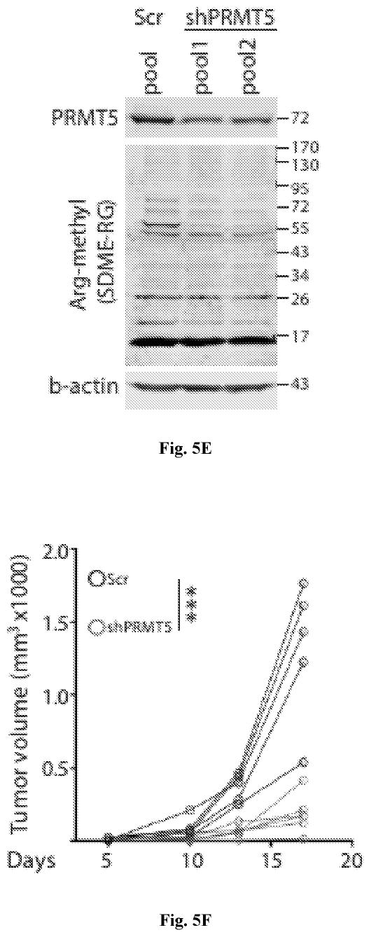

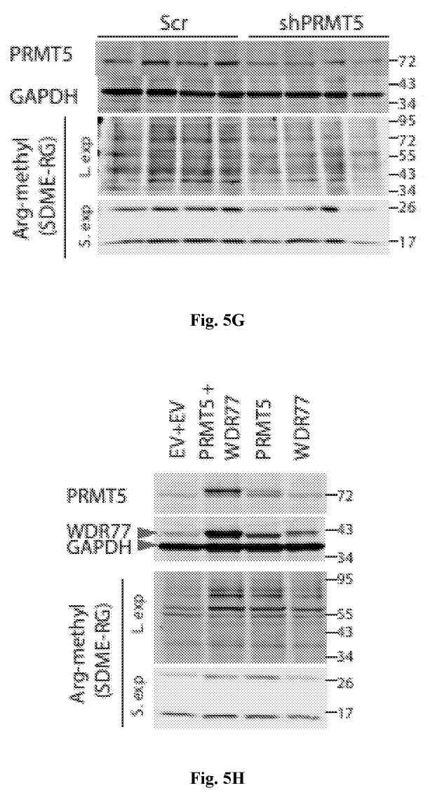

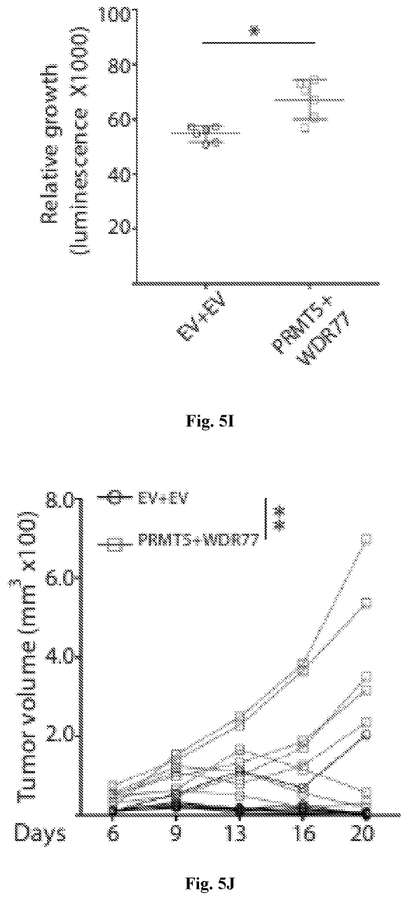

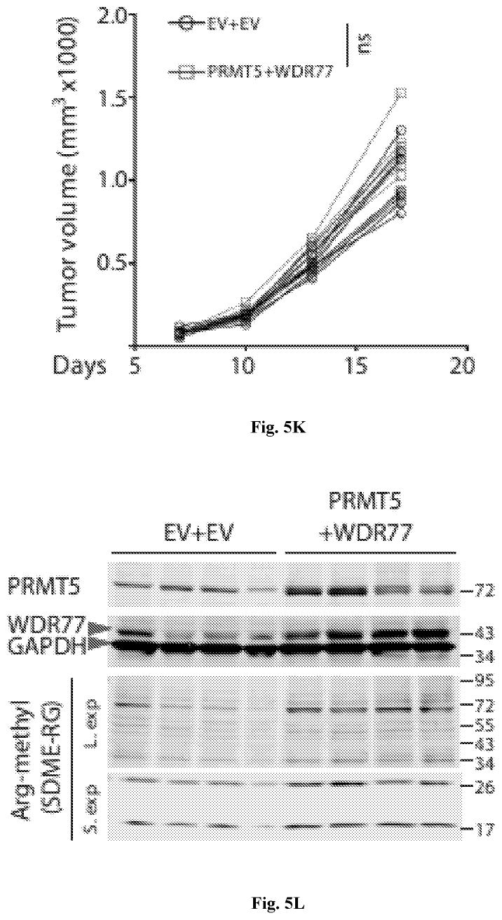

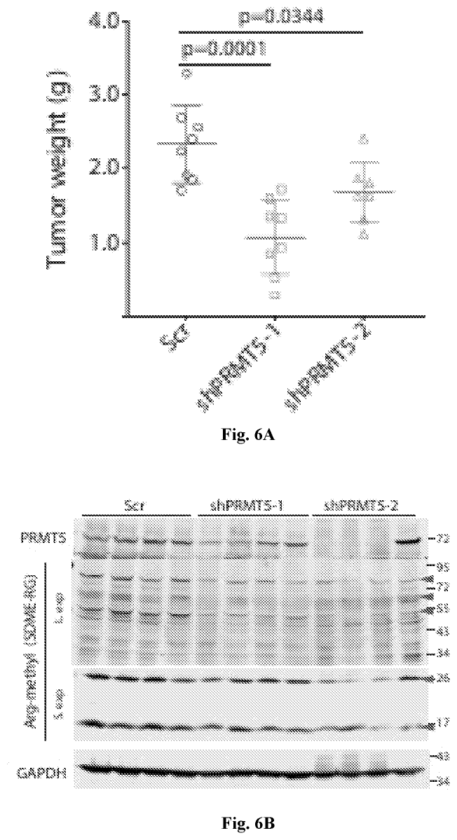

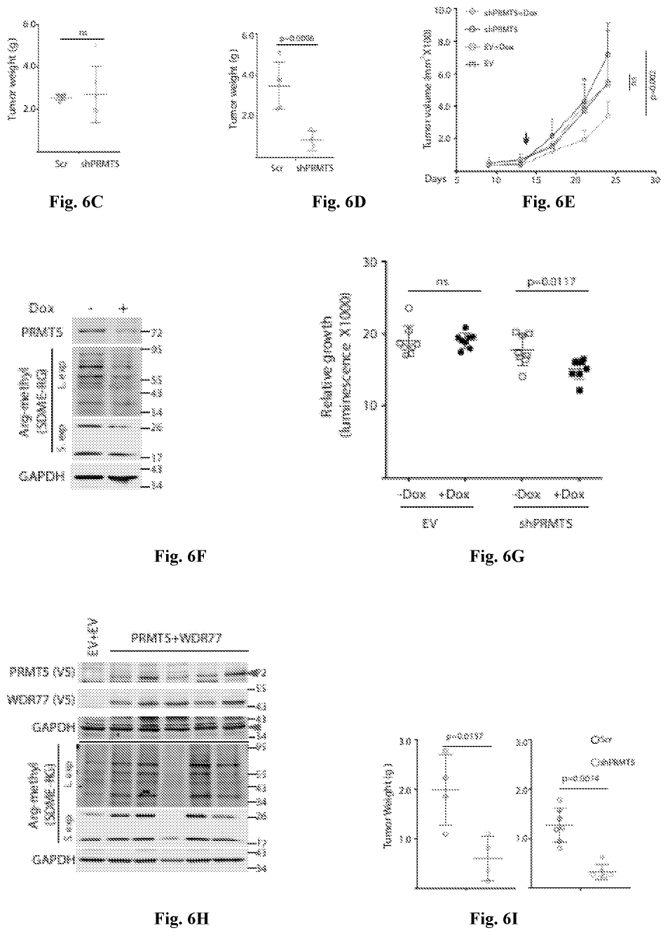

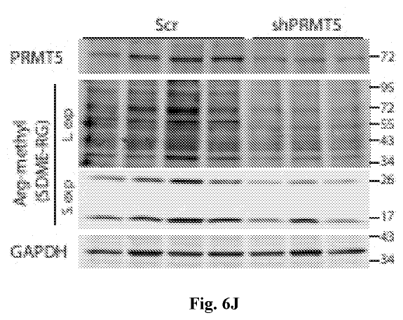

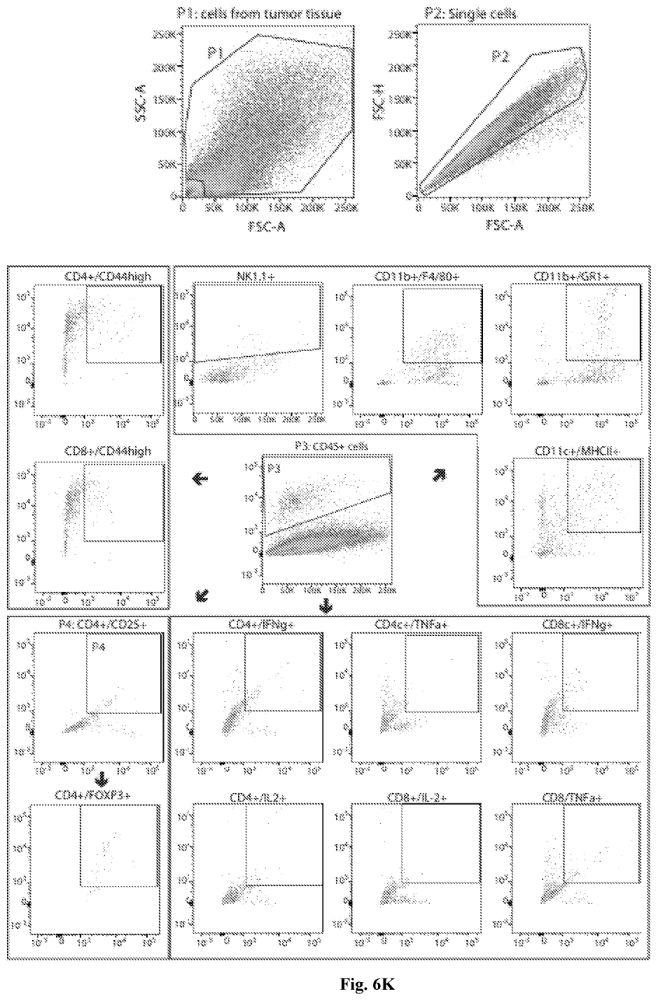

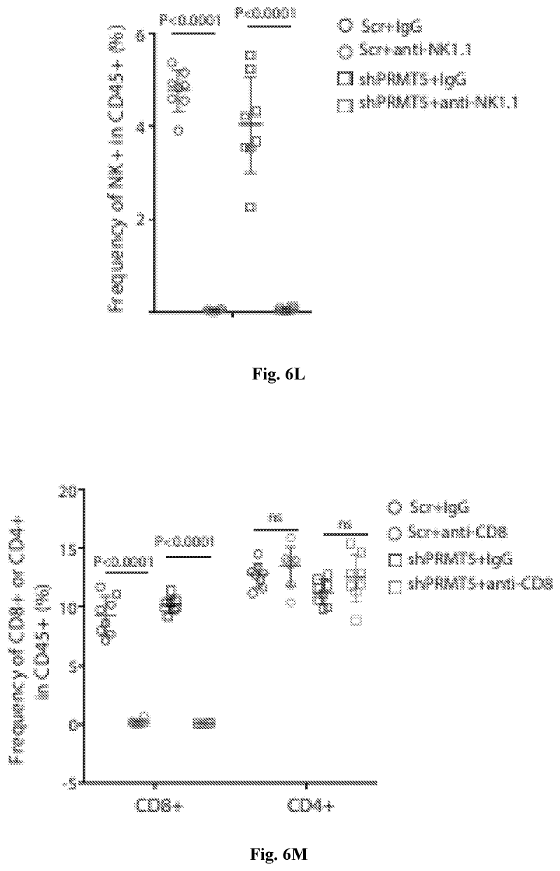

To validate in silico analyses, PRMT5 function was directly accessed in antitumor immunity by establishing B16F10 (B16) metastatic murine melanoma cells expressing either scrambled (Scr) or PRMT5-specific shRNA (KD). PRMT5 knockdown (KD) in B16 cells expressing resulted in reduced (83%-90%) PRMT5 expression and decreased PRMT5 activity (FIG. 5A). PRMT5 KD did not affect growth of melanoma cells in culture (FIG. 5B). However, inoculation of these same cultures in immunocompetent syngeneic C57BL/6 or in immunocompromised NOD-sci gamma (NSG) mice revealed important difference: in C57BL/6 mice, PRMT5 KD markedly inhibited growth (37.2-62.0% reduction in tumor volume and 28-54% reduction in tumor weight, FIG. 5C, FIGS. 6A and 6B), phenotypes not seen in B16 cells that were inoculated in the NSG mice (FIG. 5D; FIG. 6C). The ability of PRMT5 -KD B16 cells to develop tumors in immunocompromised but not immunocompetent mice suggest that that PRMT5 inhibition of melanoma growth requires an intact immune system. Transfer of B16 tumors from NSG to C57BL/6 mice resulted in growth inhibition of PRMT5 KD but not control (Scr) tumors (FIGS. 5E-G; FIG. 6D). Both the expression and activity of PRMT5 was attenuated in PRMT5 KD cells (shPRMT5 pools 1 and 2) and tumors (shPRMT5) (FIGS. 5E and 5G). Consistent with the observation in B16, PRMT5-KD in YUMM1.7 cells, which are derived from a genetically engineered murine melanoma model, (Braf^{V600E}/Pten^{-/-}/Cdkn2a^{-/-}), effectively inhibited tumor growth (52.4% in YUMM1.7, FIG. 6E), but had a lesser effect on growth of these cells in culture (14.7% in YUMM1.7; FIGS. 6F and 6G).

To substantiate the phenotypes observed upon KD of PRMT5, a gain-of-function analysis was performed using YUMMER1.7 cells, derived from UVB-irradiated YUMM1.7 cells, which increased mutation burden and antigenicity. Growth of tumors derived from YUMMER1.7 cells is inhibited in C57BL/6 but not Rag-/- mice. Given the enhanced YUMMER1.7 immunogenicity, YUMMER1.7 cells overexpressing PRMT5 or its adaptor WDR77/MEP50, which is essential for PRMT5 activity, or a combination of both, are generated to determine whether PRMT5 overexpression in YUMMER1.7 cells would antagonize immunogenicity and increase tumor growth in vivo (FIG. 5H). YUMMER1.7 cells expressing PRMT5 and WDR77 exhibited elevated expression of PRMT5 protein and correspondingly increased PRMT5 activity (FIG. 5H). Those cells also showed a moderate growth advantage in culture (relative to EV/EV controls) (FIG. 5I). Notably, in vivo, co-expression of both PRMT5 and WDR77 increased tumor growth in immunocompetent mice relative to mice harboring control YUMMER1.7 tumors (FIG. 5J; FIG. 6H). Such gain-of-function phenotypes were restricted to immune-competent animals and not seen in immune-compromised NSG mice (FIGS. 5K and 5L), suggesting that PRMT5 activity supports tumor growth by suppressing antitumor immune responses.

### Example 4. PRMT5 Controls Melanoma Infiltration of Immune Cell

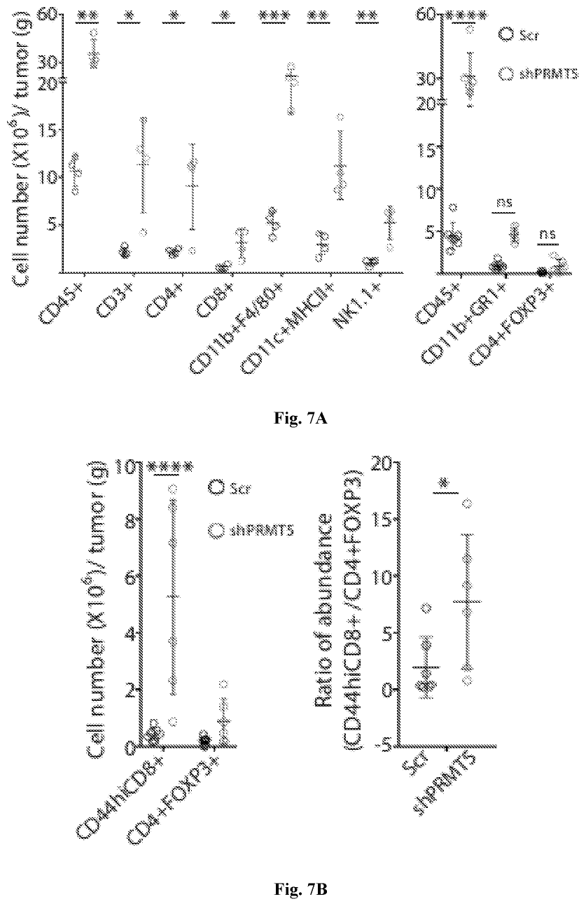

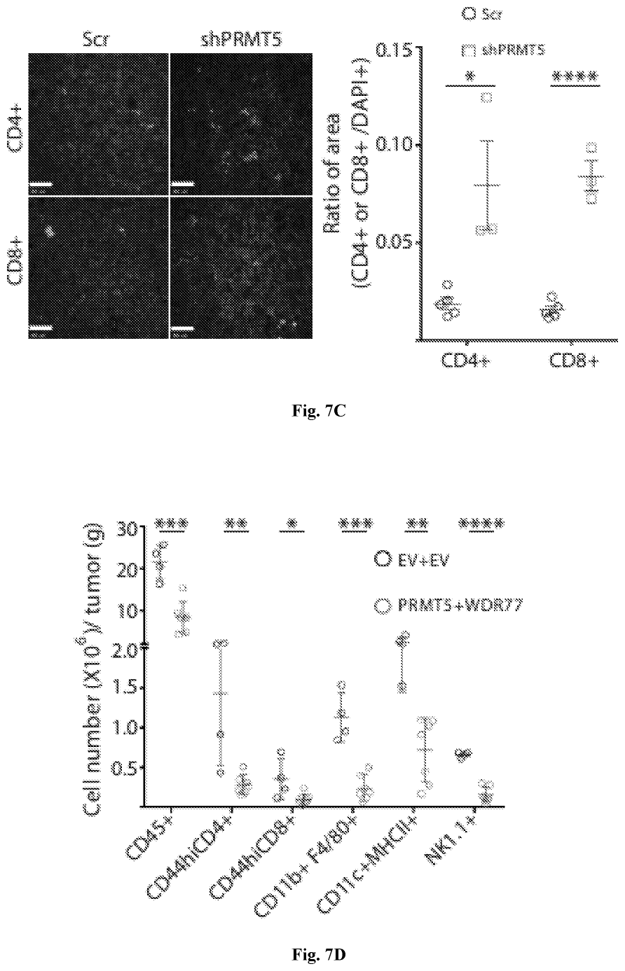

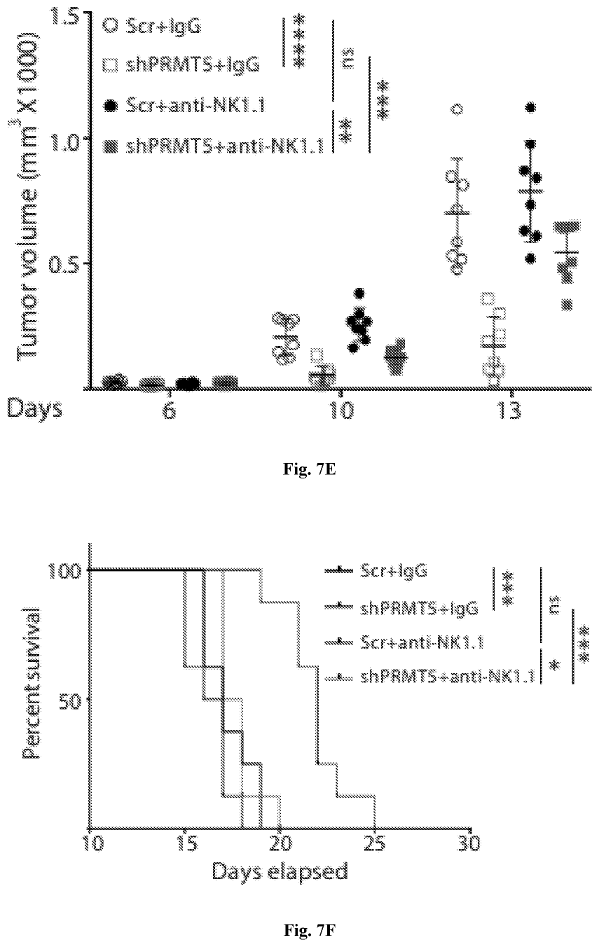

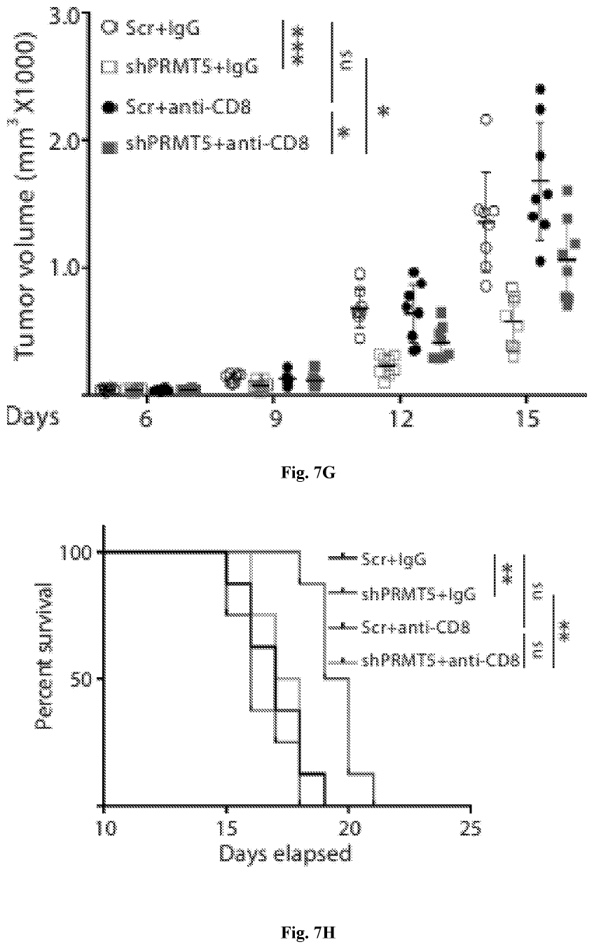

Immunocompetent mice harboring tumors derived from PRMT5 KD cells exhibited reduced tumor growth (70-75% reduction in tumor weight at day 17) relative to control mice (FIGS. 6I-J). A markedly high number of tumor-infiltrating immune cells was found in PRMT5-KD tumors, relative to Scr-expressing control tumors, which included CD45+ (3.4 fold increase, left panel), CD3+ (5.1-fold), CD4+ (4.1-fold) and CD8+ (5.1-fold) T cells, as well as natural killer (NK; 4.8-fold) cells, dendritic cells (DCs; 3.7-fold), and macrophages (4.1-fold) (FIG. 7A, FIG. 6K). Among increased CD45+ cells (12.1 fold, right panel), two immune suppressor cell types, MDSC (myeloid-derived suppressor cell, 4.9-fold) and Treg (4.5-fold), were also more abundant in PRMT5-KD tumors compared with Scr control tumors (FIG. 7A, right panel). Relative abundance of active CD8+T cells (CD44^{hi}CD8⁺) was compared to that of MDSC or Treg. Notably, relative abundance of activated CD8+ T cells (CD44^{hi}CD8⁺) to MDSC (CD11b+GR1+) or to Treg (CD4⁺FOXP3⁺) cells was higher (2.4-fold, p=0.0246 for MDSC; 7.0-fold, p=0.0383 for Treg) in PRMT5-KD compared with control tumors (FIG. 7B). Consistent with these observations, immunohistochemical analysis of tumors collected at early growth phases (day 12) confirmed increased infiltration of immune cells [CD4⁺ (4.16-fold) and CD8⁺ (5.33-fold)] in tumors harboring PRMT5 KD compared with control tumors (FIG. 7C).

Conversely, PRMT5-overexpressing YUMMER1.7 tumors (grown in C57BL/6 mice) collected at an early growth phase (day 12) showed decreased immune cell infiltration, relative to control tumors, which included decrease in CD45+(0.46-fold), CD44hiCD4+ (0.26-fold), CD44hiCD8+ (0.26-fold), natural killer cells (NK; 0.34-fold), dendritic cells (DCs; 0.32-fold), and macrophages (0.24-fold) (FIG. 7D). to substantiate the contribution of key immune infiltrated cell types on the degree of tumor growth, growth of PRMT5-KD B16 tumors was assessed following depletion of either NK or CD8 T cells. Injection of mice with neutralizing antibodies to NK or CD8 T cells restored tumor growth, that was otherwise inhibited upon PRMT5 KD, relative to IgG controls (FIGS. 7E-H; FIGS. 6L and 6M). These observations confirm overall that PRMT5 activity antagonizes immune cell infiltration in a way that allows unrestricted melanoma growth.

### Example 5. PRMT5Methylates IFI16/IFI204, a Sharpin-Interacting Intracellular DNA-Sensing Protein

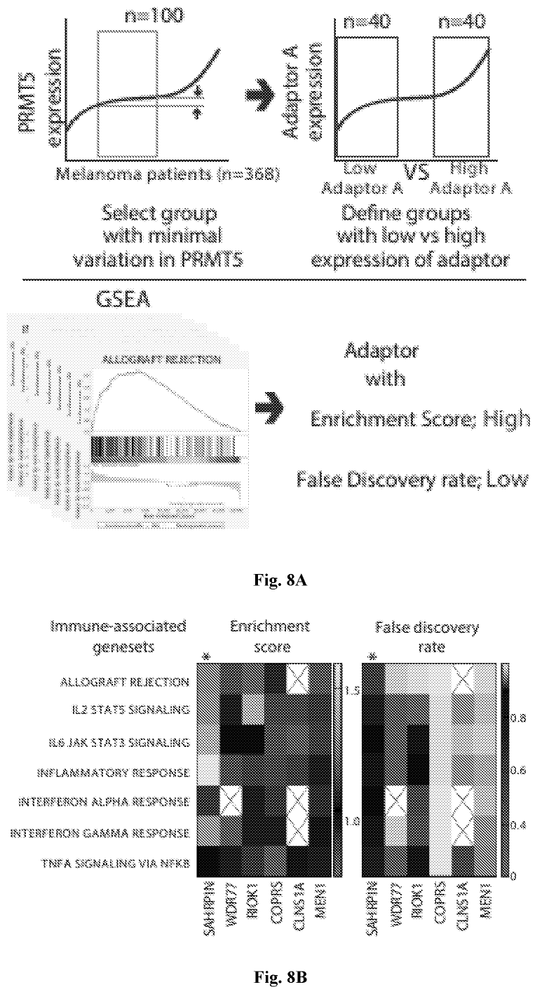

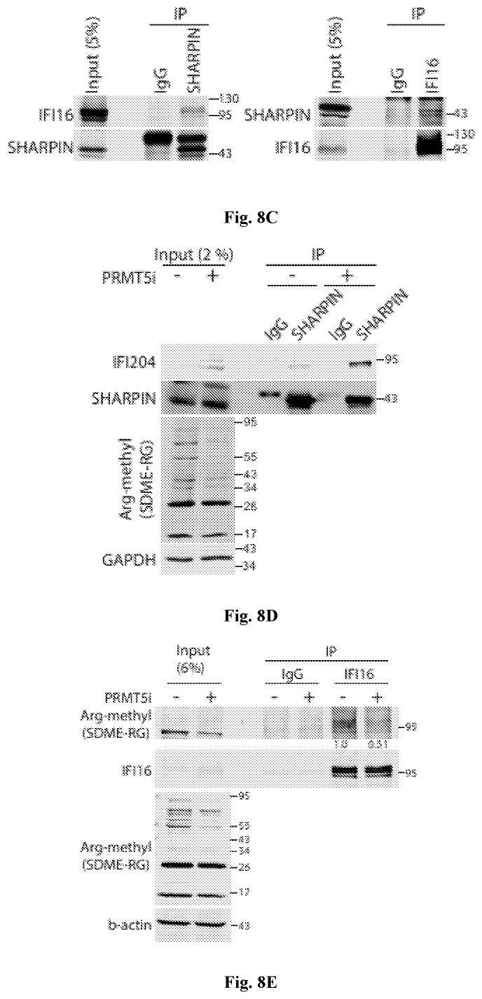

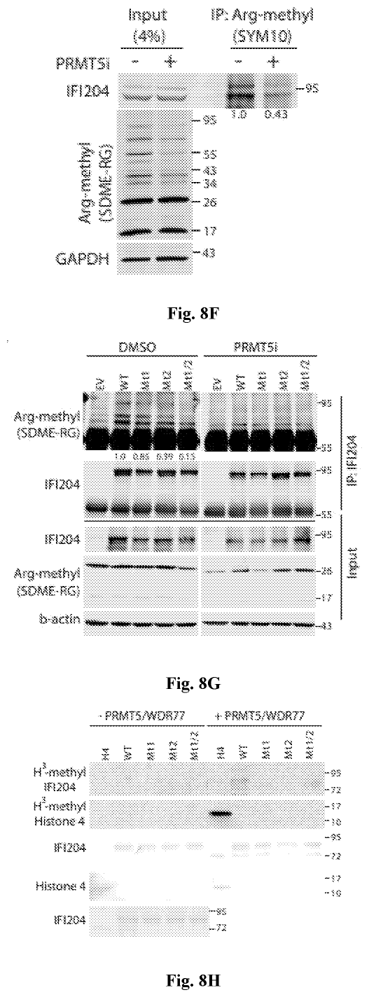

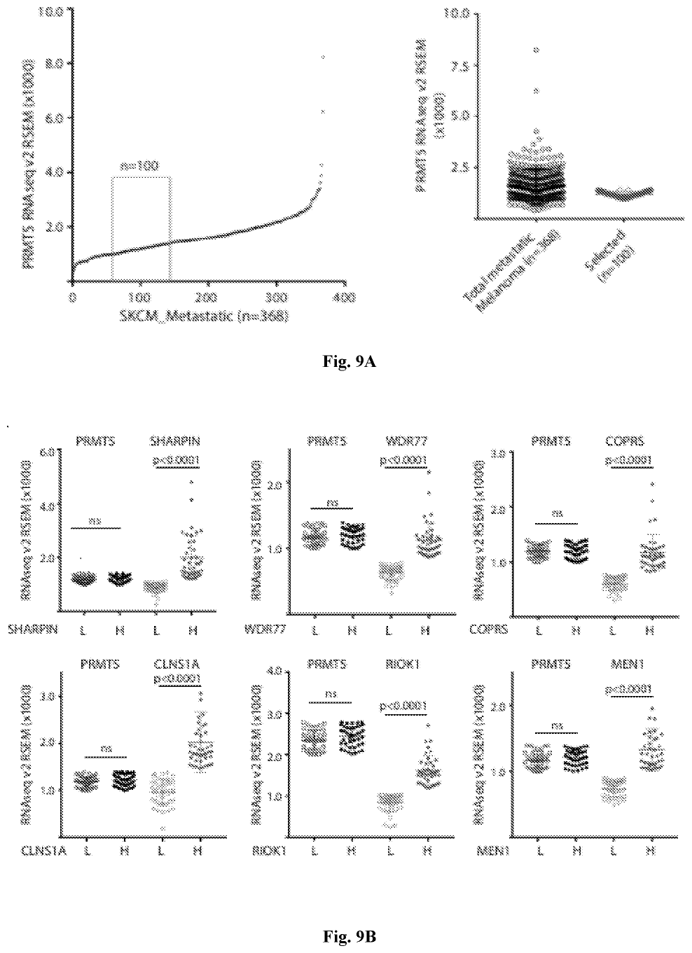

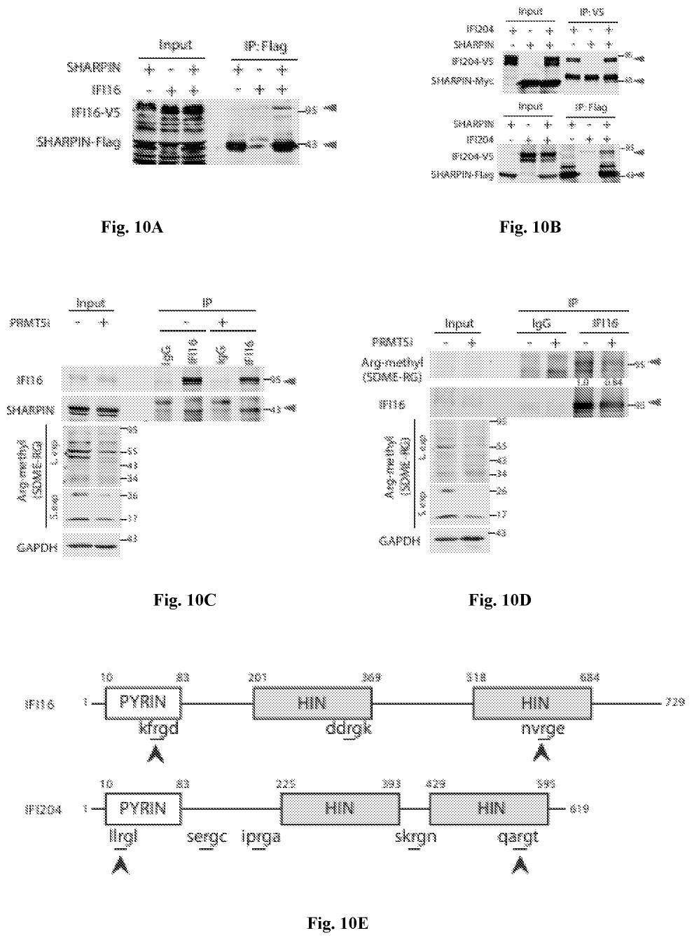

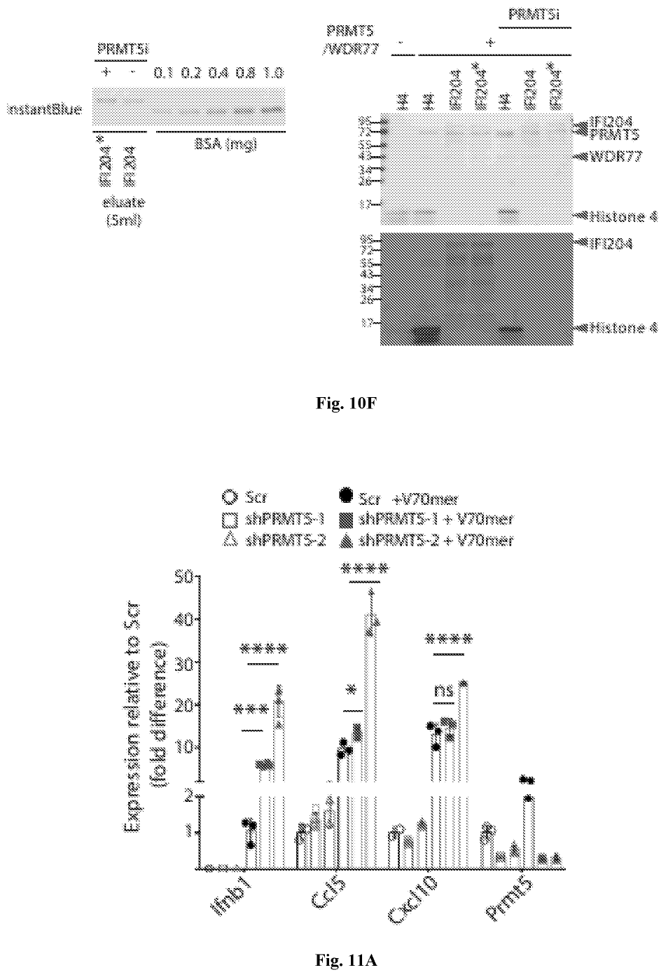

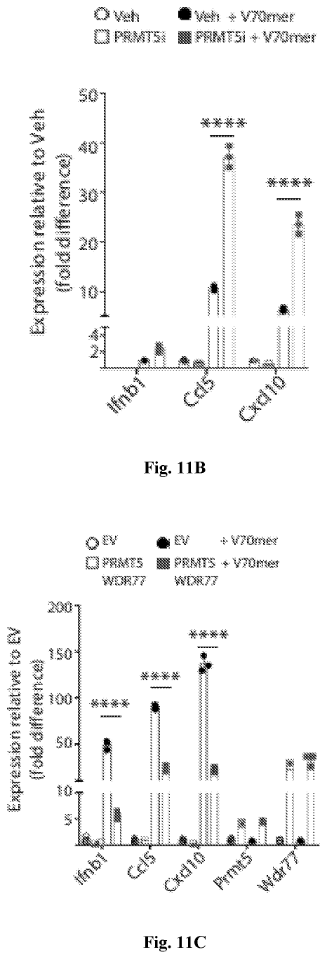

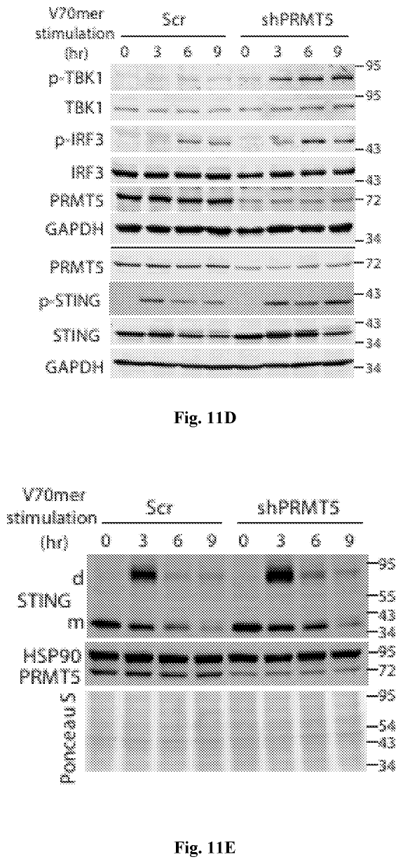

To determine whether expression of particular PRMT5 adaptors was linked with antitumor immunity, Gene Set Enrichment Analysis (GSEA) of metastatic melanoma specimens and was performed and focused on low-PRMT5 specimens, thereby excluding PRMT5 as a variable (FIG. 8A, FIG. 9A), identified an inverse correlation was identified between expression of several adaptors and the degree of anti-tumor immunity (FIG. 8A, FIG. 9B). Among those, low level of SHARPIN expression exhibited a particularly significant correlation with enrichment of immune gene signatures (FIG. 8B). SHARPIN-binding protein(s) that might serve as putative PRMT5 substrates were then searched. LC/MS/MS analysis was performed on SHARPIN interacting proteins, in the human melanoma WM115 cell line (homozygous MTAP deletion and sensitive to SHARPIN KD). Among several SHARPIN-bound proteins that could be linked with antitumor immunity (Table 4), was IFI16, a component of the intracellular DNA sensing cGAS-STING complex. IFI16 contains a DNA-binding hematopoietic interferon-inducible nuclear protein (HIN) domain (Unterholzner, et al., Nature immunology 2010;11(11):997-1004 ; Jin et al., Immunity 2012;36(4):561-71) and is implicated in controlling p53 transcriptional activity (Johnstone et al., Oncogene 2000;19(52):6033-42), in regulating cell cycle by binding to the retinoblastoma (Rb) protein (Hertel et al., Oncogene 2000;19(32):3598-608), in anti-microbial immunity by sensing cytosolic DNA (Unterholzner, et al., Nature immunology 2010;11(11):997-1004), and in inflammasome assembly through its interaction with cGAS (cyclic guanosine-monophosphate adenosine-monophosphate synthase) and STING (stimulator of interferon genes)(Almine et al., Nature communications 2017;8:14392; Jonsson et al., Nature communications 2017;8:14391, each incorporated herein by reference in its entirety). Interaction of IFI16, or its murine homologue IF204, with SHARPIN was confirmed in series of IP reactions (FIGS. 8C and 8D; FIGS. 10A-C). Degree of interaction between IFI16 and SHARPIN was enhanced in A375 melanoma cells(which harbor intact MTAP expression and higher PRMT5 activity) upon PRMT5 inhibition (using pharmacological PRMT5 inhibitor, EPZ015666) (FIG. 10C), pointing to the possibility that PRMT5 activity may limit IFI16 binding. To substantiate this observation, changes in IFI16 methylation following PRMT5 inhibition was assessed. A375 melanoma cells treated with the PRMT5 inhibitor EPZ015666, revealed a 50% decrease in IFI16 methylation, compared with 15% decrease in IFI16 methylation in WM115 cells, which are MTAP- deleted and thus have lower basal level of PRMT5 activity (FIG. 8E; FIG. 10D). Likewise, murine IFI16 homolog IFI204 exhibited reduced methylation (by 57%) in EPZ015666 treated B16 cells (FIG. 8F), which coincided with stronger interaction with SHARPIN, as seen in A375 cells (FIG. 8D). Search for consensus RG motifs which harbor arginine residues that could be methylated (Tamiya et al., The Journal of clinical investigation 2018;128(1):517-30; Jansson et al., Nature cell biology 2008;10(12):1431-9; Clarke et al., Molecular cell 2017;65(5):900-16), each incorporated herein by reference in its entirety) identified Arg12 located within the N-terminal PYRIN (protein-protein interaction) domain, and Arg538, located in the C-terminal HIN (DNA-binding) domain in the murine IFI204 protein (FIG. 10E). mutating either

Arg12, Arg538, or both Arg12 and Arg538 were mutated to alanine (A) in IF204and Arg methylation by PRMT5 was evaluated (FIG. 8G). Mutation of either Argl2Ala (R12A) or Arg538Ala (R538A) reduced degree of IFI204 whereas mutation of both residues further lowered the extent of IF204 methylation, confirming that both the R12 and R538 residues are PRMT5 methylation sites. VS-tagged IFI204 proteins (WT, R12A and R538A) were immunopurified from HEK293T cells and subjected to in vitro methylation reaction using recombinant PRMT5 /WDR77 (FIG. 8H; FIG. 10F). While IFI204 WT protein was methylated by PRMT5 /WDR77, this methylation that was no longer seen in the presence of PRMT5i (FIG. 10F). Immunopurified WT IFI204, but not a mutant form lacking either Arg12, Arg538 or both, was methylated in vitro by recombinant PRMT5 (FIG. 8H).

TABLE 4
LC/MS-MS identifies SHARPIN-interacting proteins
including IFI16 as well as known interacting
proteins, RNF31 and RBCK1 (components of LUBAC)

| Protein ID | Name | Protein ID | Name |
|---|---|---|---|
| Q9H0F6 | SHARPIN | O14950 | MYL128 |
| P35579 | MYH9 | P19105 | MYL12A |
| P49327 | FASN | P38646 | HSPA9 |
| P35580 | MYH10 | P04406 | GAPDH |
| P09211 | GSTP1 | P06576 | ATP5B |
| P08670 | VIM | Q00839 | HNRNPU |
| Q00610 | CLTC | Q16875 | PFKFB3 |
| P11142 | HSPA8 | P98175 | RBM10 |
| P14618 | PKM | P06396 | GSN |
| P07237 | P4HB | Q8WWY3 | PRPF31 |
| P07437 | TUBB | P13010 | XRCC5 |
| Q9UJS0 | SLC25A13 | Q14204 | DYNC1H1 |
| P21333 | FLNA | Q16666 | IFI16 |
| O60825 | PFKFB2 | P09874 | PARP1 |
| P10809 | HSPD1 | P40967 | PMEL |
| P11021 | HSPA5 | Q96T60 | PNKP |
| O14744 | PRMT5 | P12235 | SLC25A4 |
| P12956 | XRCC6 | Q15084 | PDIA6 |
| Q96EP0 | RNF31 | Q9BYM8 | RBCK1 |

### Example 6. PRMT5-Dependent IFI16/IFI204 Methylation Attenuates dsDNA-Induced TBK1-IRF3 Activation and Interferon and Chemokine Production

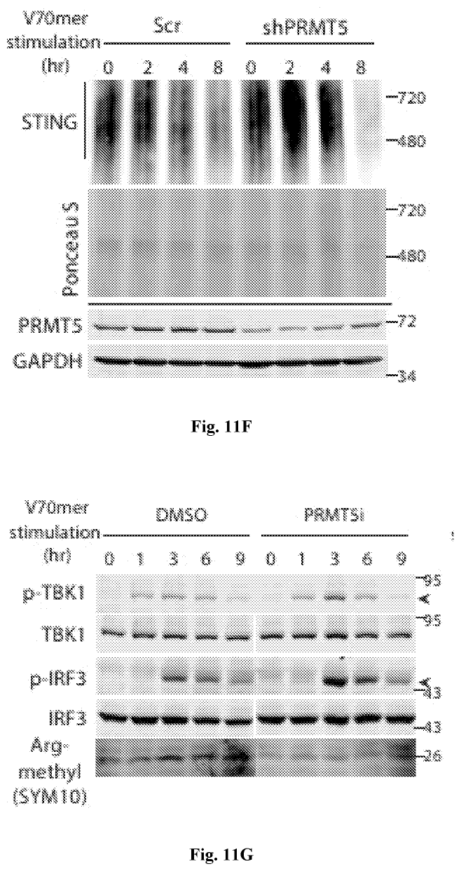

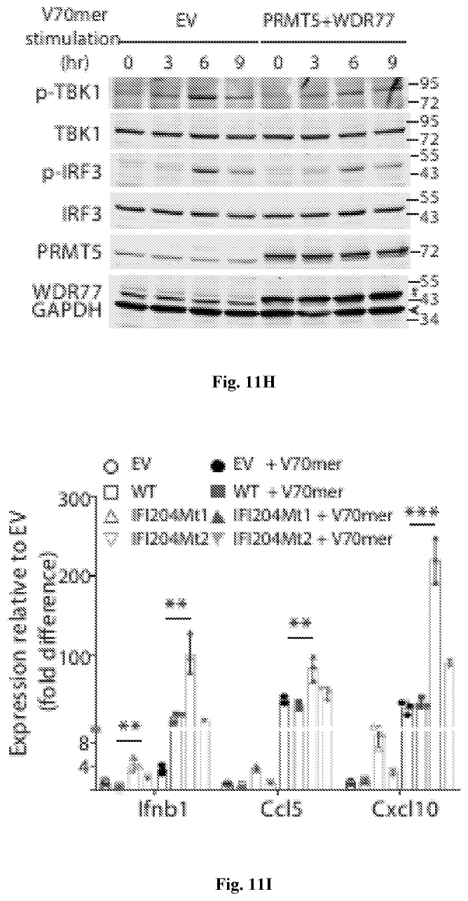

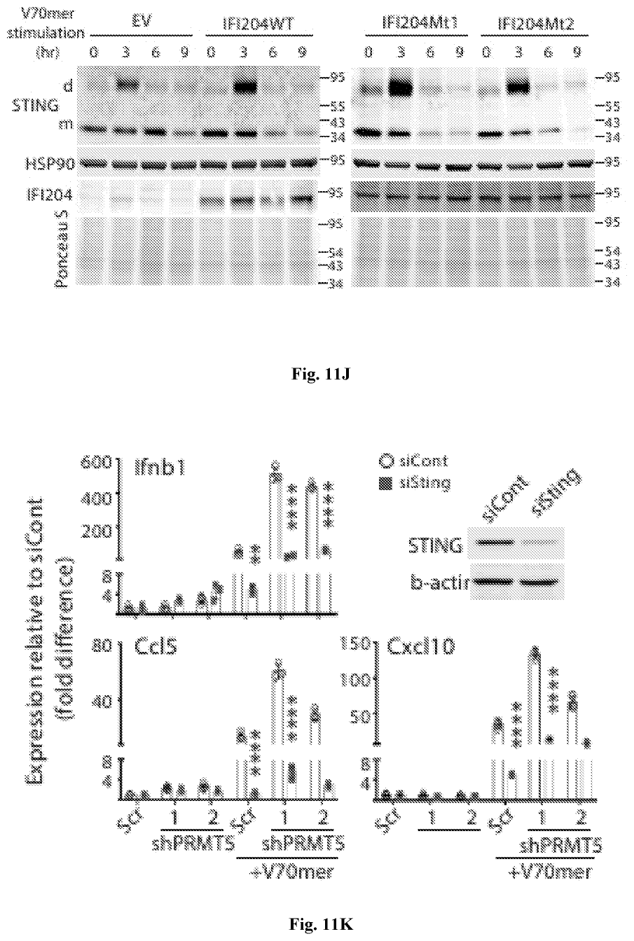

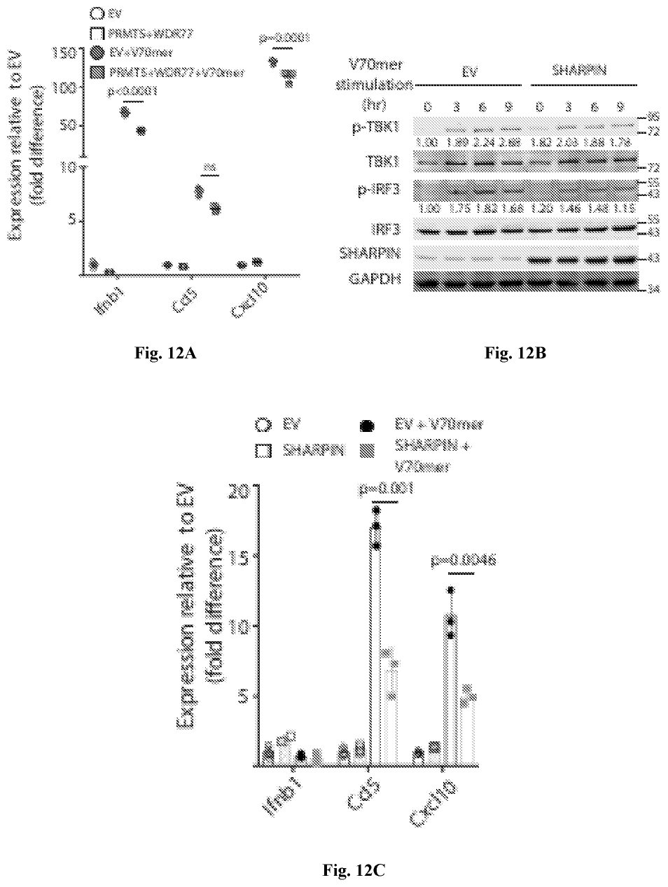

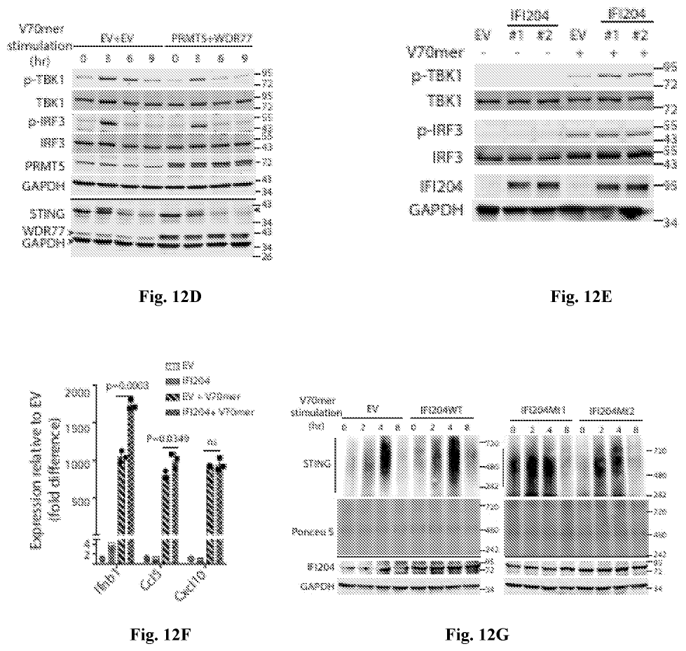

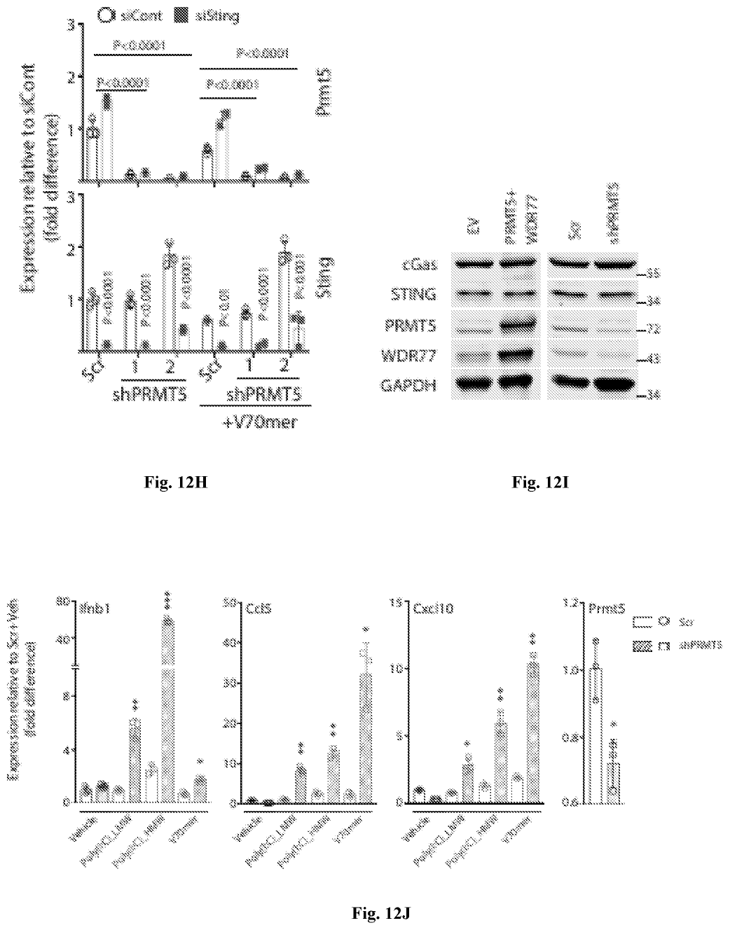

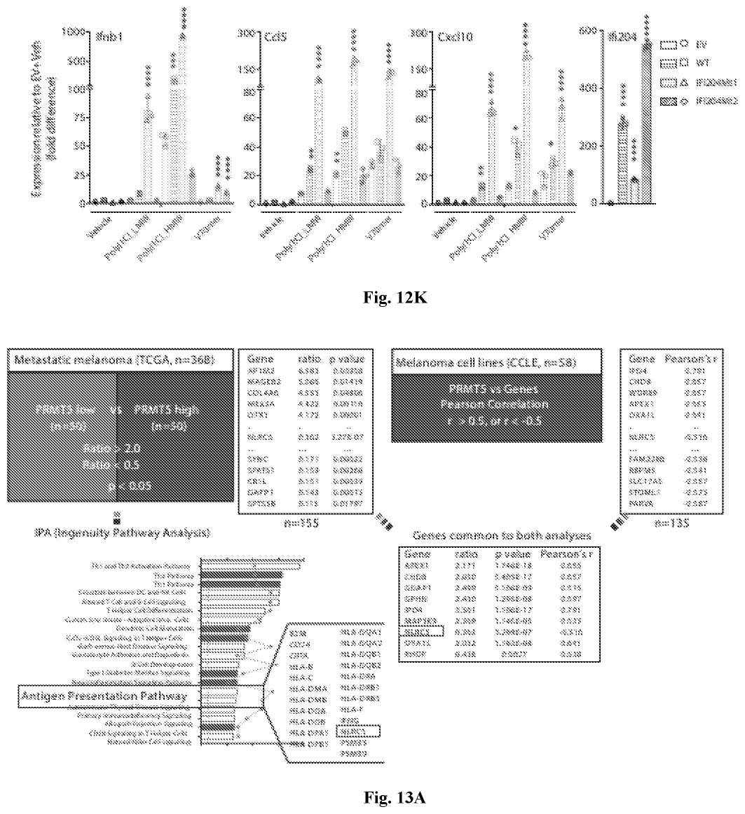

IFI16/IFI204 binding to intracellular dsDNA induces expression of Ifnb1,chemokines Cc15, and Cxcl10 (Almine et al., Nature communications 2017;8:14392; Unterholzner et al., Nature immunology 2010;11(11):997-1004; Josson et al., Nature communications 2017;8:14391, each is incorporated herein by reference in its entirety). Given that PRMT5 methylates IFI16/IFI204, IFI204-dependent chemokine induction upon PRMT5 methylation was examined. Indeed, attenuating PRMT5 expression (by shPRMT5) or activity (with EPZ015666) increased expression of Ifnb1, Cc15 and Cxcl10 following stimulation with 70 base-pair dsDNA [referred to as V70-mer; (29)] (FIGA. 11A and B). Conversely, PRMT5 overexpression decreased dsDNA-stimulated expression of all three genes both in B16 and YUMMER1.7 cells (FIG. 11C; FIG. 12A) SHARPIN overexpression in B16 cells also attenuated intracellular DNA-mediated activation of the TBK1-IRF3 pathway and subsequent expression of Ifnbl, Cc15 and Cxcl10 (FIG. 12B and 12C). Conversely, genetic inhibition of PRMT5 in B16 cells augmented dsDNA-induced activation of TBK1-IRF3 as reflected by levels of STING phosphorylation, dimerization and polymerization (FIGS. 11D-F). Similarly, pharmacological inhibition of PRMT5 in B16 melanoma activated TBK1-IRF3 signaling (FIG. 11G). These observations were further substantiated by the finding that ectopic expression of PRMT5/WDR77 in B16 or YUMMER1.7 cells effectively decreased TBK1-IRF3 activation (FIG. 11H; FIG. 12D). Changes in TBK-IRF3 signaling following DNA stimulation was monitored to substantiate the importance of IFI204 methylation, which is part of the cGAS-STING pathway. Ectopically expressed IFI204 in B16 cells activated TBK1-IRF3 signaling (FIG. 12E) and increased the expression of Ifnb1 and Cc15 (FIG. 12F), following dsDNA treatment, compared to cells that expressed control plasmid. Notably, expression of methylation-defective R12A IFI204 (IFI204Mt1), but not R538A (IFI204Mt2), further increased TBK1-IRF3-mediated expression of downstream genes relative to changes seen following expression of wildtype (WT) IFI204 (FIG. 11I). consistent with these observations, IFI204Mt1 expression, but not that of IFI204Mt2, increased STING dimerization and polymerization following dsDNA-stimuli (FIG. 11J and FIG. 12G), suggesting a critical role of Arg12 methylation of IFI204 in the activation of STING pathway by dsDNA-stimuli. In agreement, siRNA-mediated STING knockdown markedly reduced, albeit not completely, activation of Ifnb1, Cc15 and Cxcl10 seen after PRMT5-downregulation (FIG. 11K and FIG. 12H). Of note, changes in PRMT5 expression did not alter cGAS or STING expression (FIG. 12I), suggesting that PRMT5 limits the activation but not the expression cGAS/STING pathway components.

PRMT5 activity was accessed to determine whether PRMT5 activity might regulate RIG-I/TLR3-mediated activation of a type I interferon response by dsRNA. B16 melanoma cells treated with the RIG-I/TLR3 agonist poly(I:C) induced Ifnb 1 and chemokine expression, which was significantly enhanced upon PRMT5 KD r (FIG. 12J). Surprisingly, B16 melanoma cells overexpressing IFI204Mt1, but not IFI204Mt2, exhibited increased induction of Ifnbl and chemokine expression by poly (I:C) relative to levels seen in WT cells (FIG. 12K). These observations reveal an unexpected role for PRMT5/IFI16 in dsRNA-induced activation of type I interferon response and suggest that PRMT5 controls dsDNA-induced STING-dependent and dsRNA-induced activation of the type I interferon response.

### Example 7. PRMT5Regulates Antigen Presentation by Controlling NLRC5 Expression

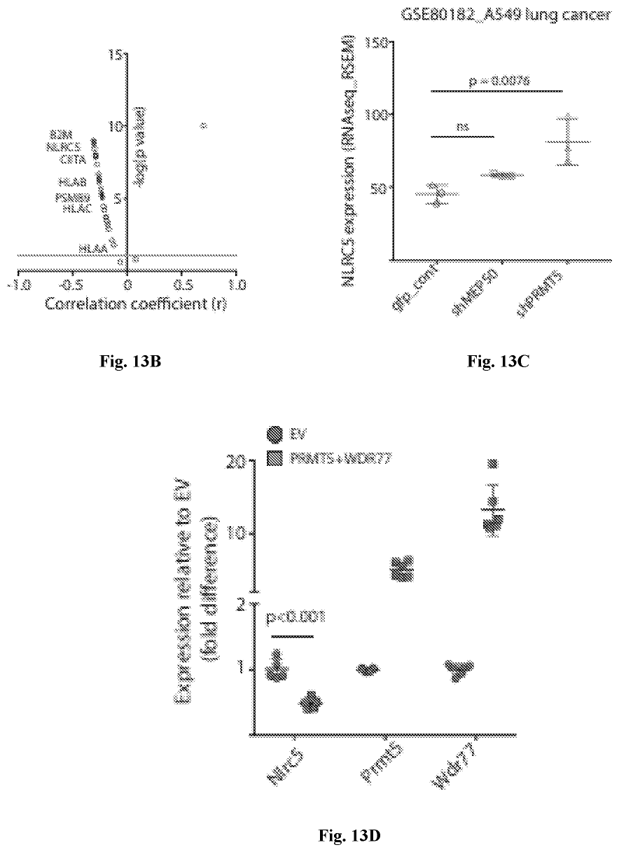

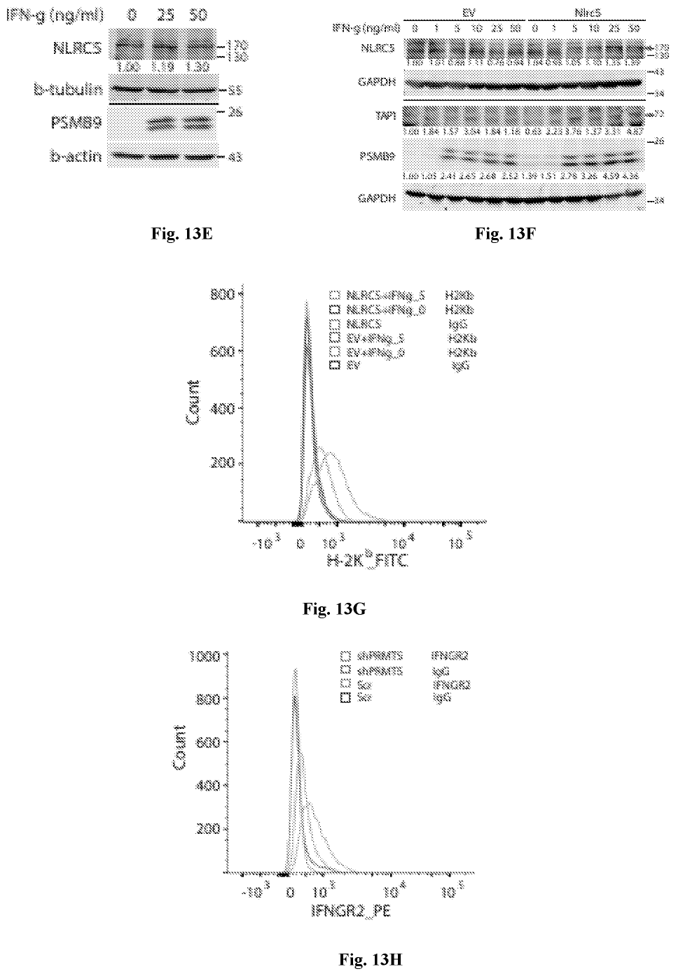

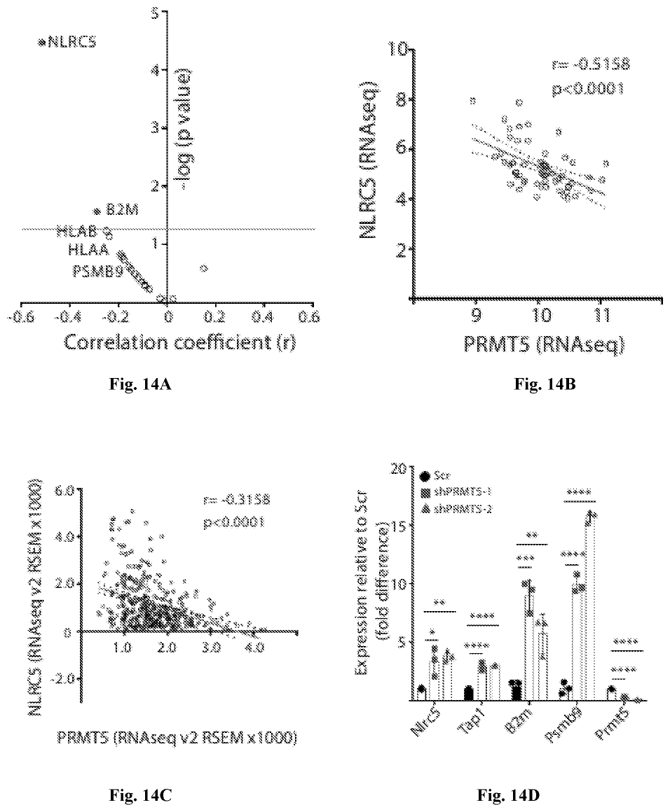

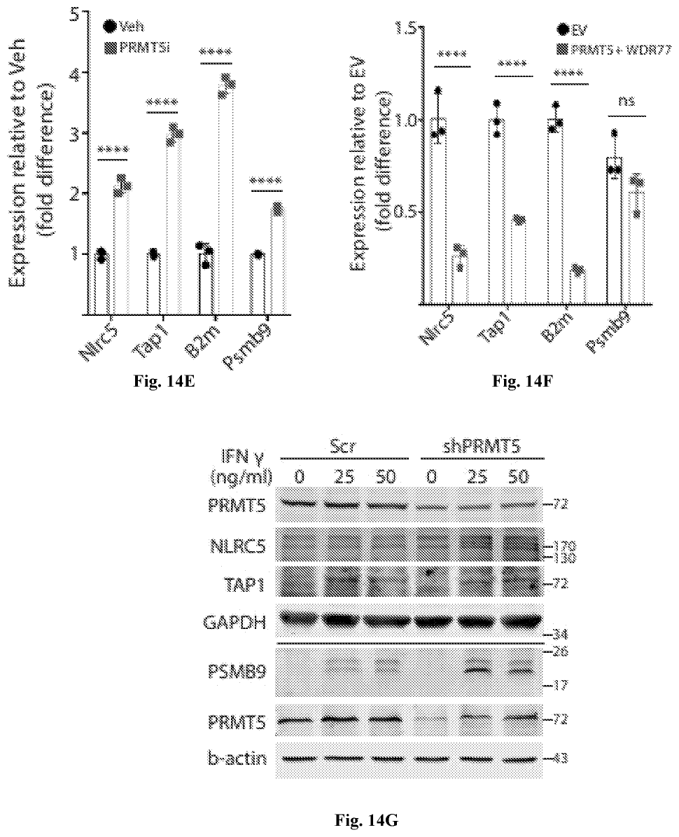

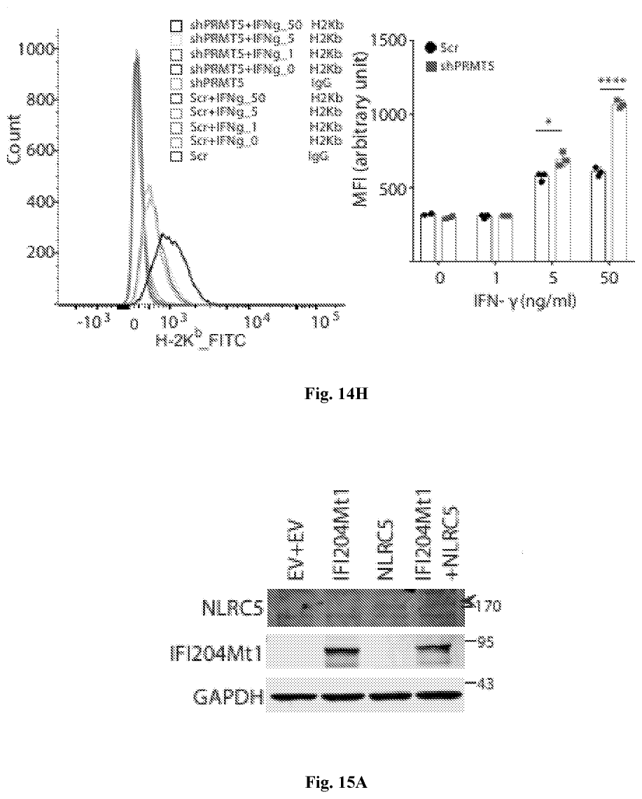

To search for PRMT5-regulated genes that contribute to tumor immune responses, both the TCGA metastatic melanoma dataset (n =368) and the Cancer Cell Line Encyclopedia (CCLE) (Barretina et al., Nature 2012; 493 (7391): 603-7) (n=58) were surveyed. Of PRMT5 co-regulated genes (155 genes from TCGA and 135 genes from CCLE) (FIG. 13A), nine were common to both datasets, of which one—the transcriptional activator NLRC5 -(NLR Family CARD Domain Containing 5), a transcriptional activator of genes which had been indicated in regulating MHC class I gene expression(Biswas et al., J. of Immunol. 2012; 189(2): 516-20; Kobayashi et al., Nature reviews immunology 2012; 12(12): 813-20). NLRCS, along with B2M, HLA-A, -B, -C, PSMB9, are implicated in antigen presentation, a pathway predicted by IPA analysis (antigen presentation pathway, log (p-value)=−13.9; FIG. 2B). NLRCS expression was inversely correlated with PRMT5 expression in both the CCLE (r=−0.516, p<0.0001) and TCGA (r=−0.3158, p<0.0001) datasets (FIGS. 14A-C; FIG. 13B). Increased NLRCS expression was also seen in lung cancer cells that were subjected to PRMT5 inhibition (Chen et al., Oncogene 2017; 36(3): 373-86), consistent with observations (FIG. 13C). Given that downregulation of proteins involved in MHCI- and/or MHCII-mediated antigen presentation is a common mechanism of immune evasion by cancer cells, PRMT5 activity was examined to determine whether its activity inhibited of NLRCS expression and/or altered its regulation of genes implicated immune evasion. Genetic (shRNA) or pharmacological (using MTA) inhibition of PRMT5 increased basal Nlrc5 expression and that of Nlrc5 target genes implicated in antigen presentation (MHCI) and processing (such as Tap1, B2m, and Psmb9; (FIGS. 14D, 14E). In contrast, ectopic expression of PRMT5 /WDR77 in B16 or YUMMER1.7 melanomas decreased expression of NLRCS and its target genes (FIG. 14F, FIG. 13D). In agreement with earlier reports(Biswas et al., J. of Immunol. 2012; Rodriguez et al., Oncoimmunology 2016 5(6):e1151593, incorporated herein by reference in its entirety), overexpression of NLRCS or interferon-gamma stimuli, which induces expression of NLRC5, increased expression of PSMB9 and surface expression of MHCI (H-2Kb) in B16 cells (FIGS. 13E-G). Importantly, PRMT5 KD in B16 cells NLRCS and PSMB9 expression following interferon treatment (FIG. 14G) with a concomitant increase in surface MHCI expression (FIG. 14H) PRMT5 loss did not alter expression of IFN-gamma-receptor (FIG. 13H), suggesting that changes observed here are not due to changes in interferon gamma receptor expression. These findings suggest that PRMT5 controls MHCI expression and antigen presentation via its effect on NLRCS transcription.

### Example 8. IFI204 and NLRC5 Expression Inhibits in Vivo Growth of Mouse Melanoma

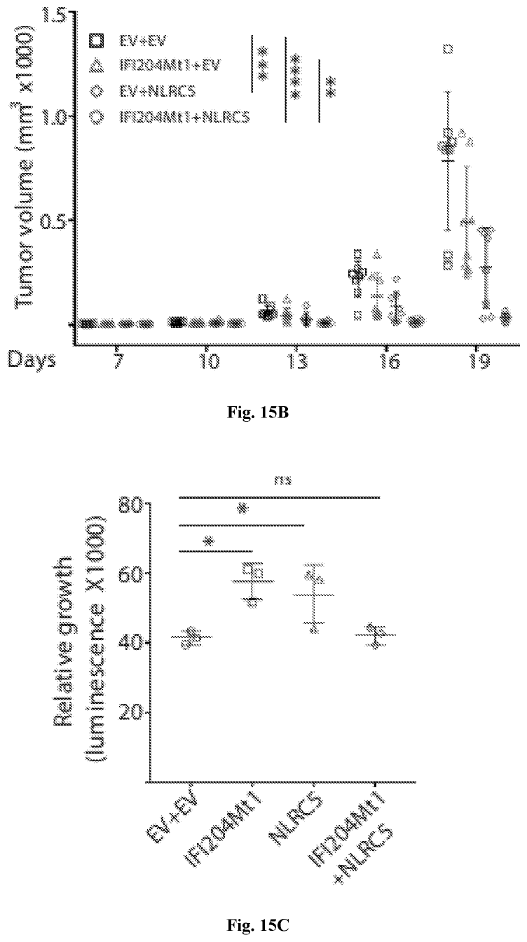

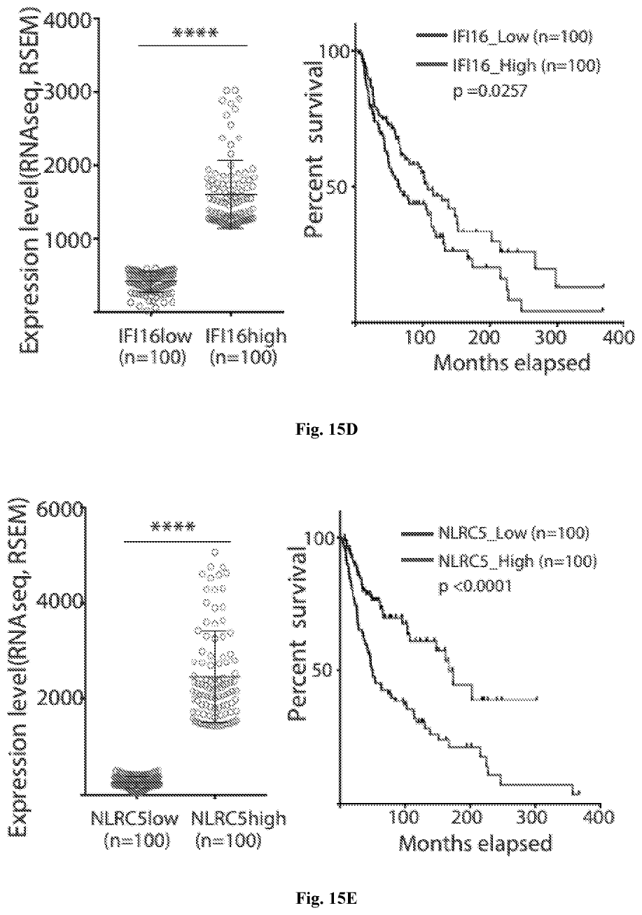

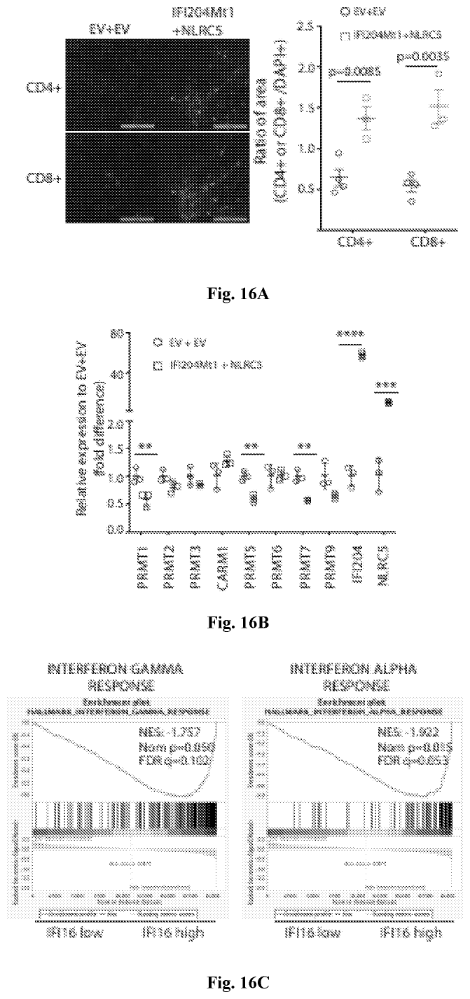

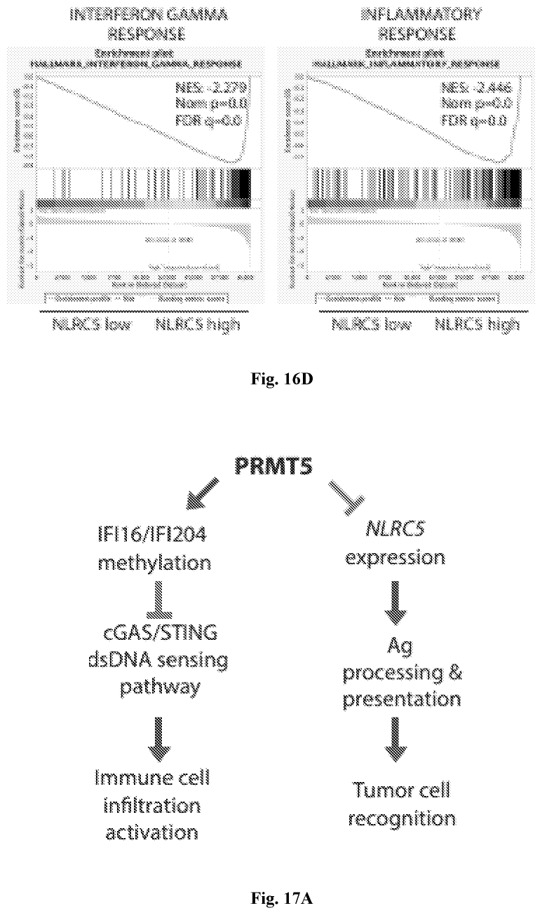

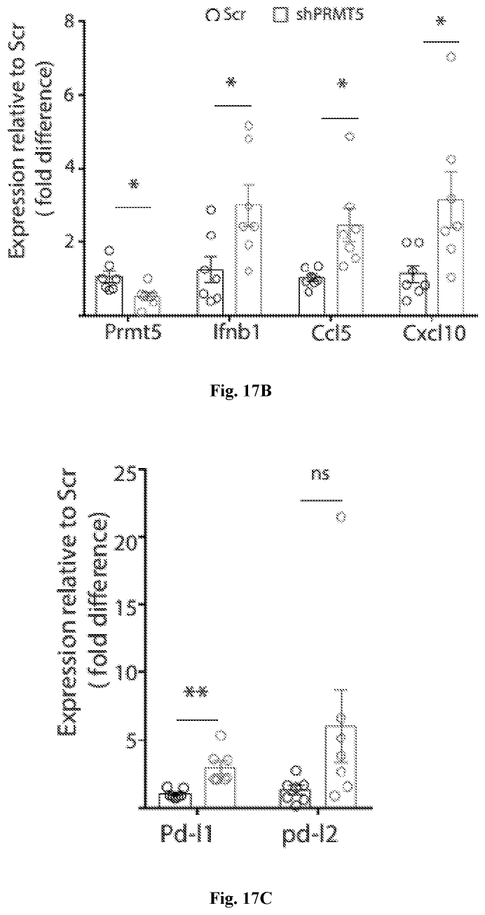

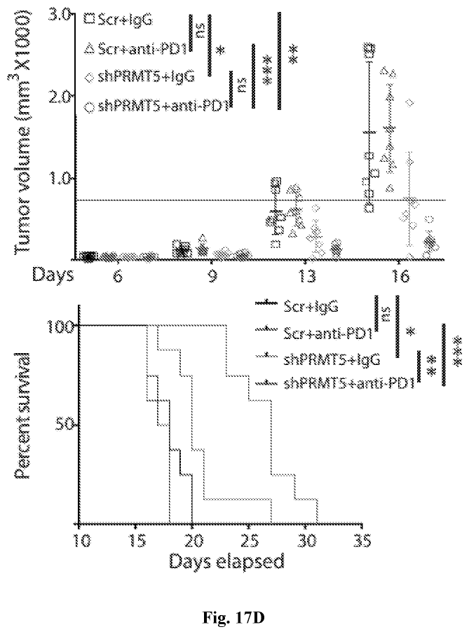

Given that PRMT5 suppressed IFI204 activity and NLRCS expression, phenocopy of IFI204Mt1 and/or NLRCS expression of PRMT5 depletion was tested. To do so, mouse melanoma cells (B16F10) expressing methylation defective IFI204 (IFI204Mt1), murine NLRCS, or both were established (FIG. 15A). Ectopic expression of NLRCS alone, but not of IFI204Mt1 alone, significantly inhibited tumor growth in mice. However, the degree of tumor growth suppression in vivo was enhanced in melanoma expressing NLRC5 and IFI204Mt1 relative to NLRC5 alone, supporting that both pathways mediate antitumor immunity, as seen upon PRMT5 inhibition (FIG. 15B). When cells were grown in culture, however, growth suppression was not observed in lines ectopically expression IFI204Mt1 and/or NLRC5, consistent with changes observed for PRMT5 inhibition, and supporting the ideal that in vivo these factors function in tumor immune recognition (FIG. 15C).Of note, immunohistochemical analysis of tumors (collected at day 12) showed increased infiltration of immune cells [CD4+ (2.13-fold) and CD8+ (2.80-fold)] in tumors expressing NLRC5 and IFI204Mt1 relative to control tumors (FIG. 16A). Moreover, expression of PRMT1, PRMT5 and PRMT7 decreased in cells expressing IFI204Mt1 plus NLRC5, suggesting a possible feed-forward mechanism limiting PRMT5 or other PRMTs activity (FIG. 16B).

### Example 9. Expression of IFI16 and NLRC5 is Associated with Prolonged Patient Survival

Possible association between PRMT5 downstream regulators IFI16 and NLRC5 and melanoma patient survival was examined. Analysis of 200 melanoma specimens [IFI16-or NLRC5-low (n=100) or IFI16- or NLRC5-high (n=100) in the TCGA dataset (n=368 metastatic melanomas) revealed significantly prolonged survival of melanoma patients whose tumors exhibit higher expression of either IFI16 (p=0.0257) or NLRC5 (p<0.0001) (FIG. 15D and 15E). Correspondingly, higher expression of IFI16 or NLRC5 coincided with enrichment of an immune gene signature (FIG. 16C and 16D), supporting the notion that IFI16 and NLRC5 are essential for PRMT5-dependent control of the tumor immune response. Example 10. PRMT5 inhibition enhances immune checkpoint therapy in a murine melanoma model.

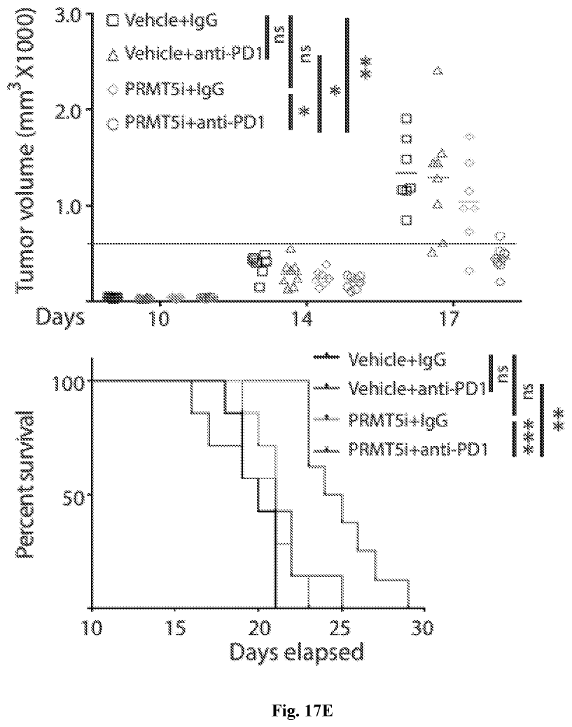

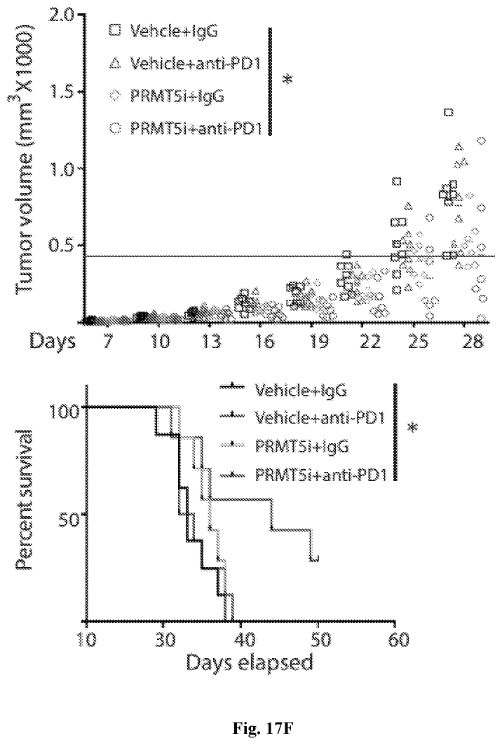

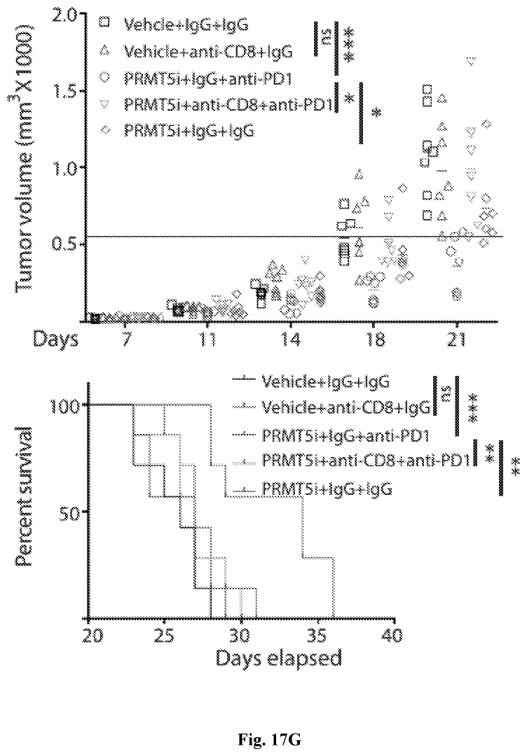

The findings disclosed herein provide the basis for a model highlighting the role of PRMT5 as a suppressor of antitumor immune response, which is achieved by limiting infiltration/activation of immune cells and tumor cell recognition by immune cells, as shown in (FIG. 17A). Consistent with this model, PRMT5 KD tumors expressed elevated levels of Ifnb1, Cc15 and Cxcl10 (FIG. 17B) and higher levels of Pd-11(Cd274), an immune check-point ligand, relative to control tumors (FIG. 17C). Since enhancing the immune response to so-called “cold” tumors could augment ICT effectiveness, whether PRMT5 inhibition would augment the effectiveness of immune checkpoint therapy (ICT) was tested. PRMT5 KD (shPRMT5+IgG) significantly attenuated B16 tumor growth in 6 of 8 mice compared with 1 of 8 in the control group (Scr+IgG) (p=0.0406, Fisher's exact test) (FIG. 17D, upper panel).

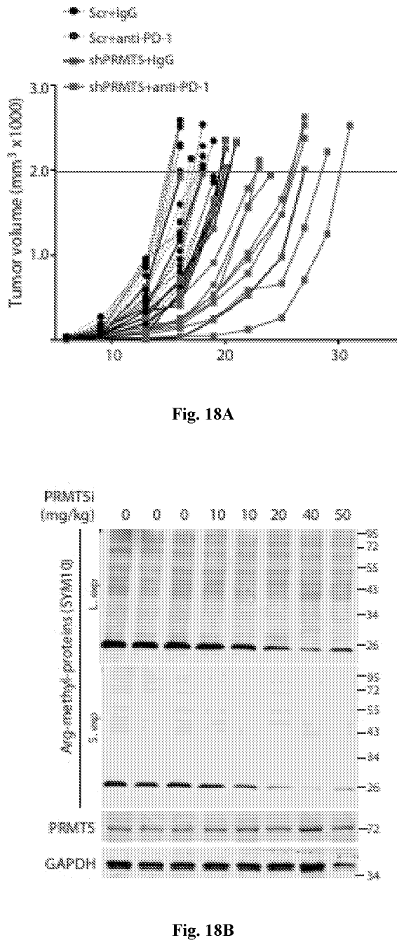

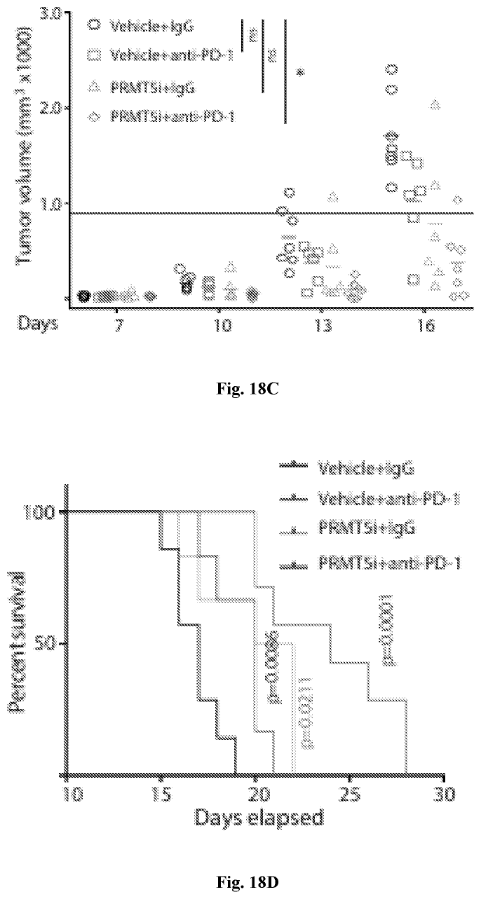

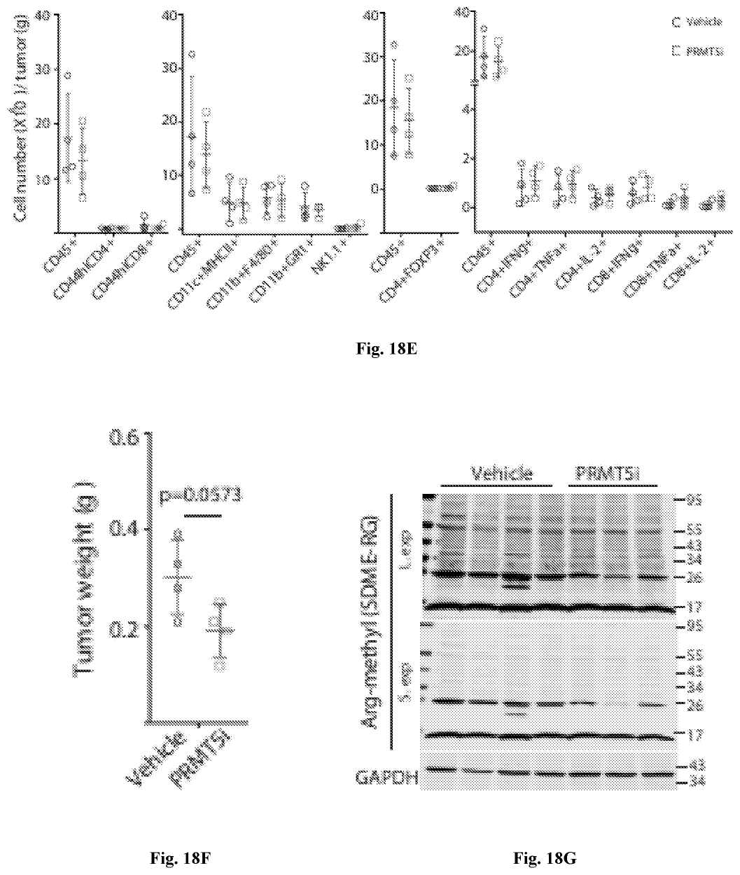

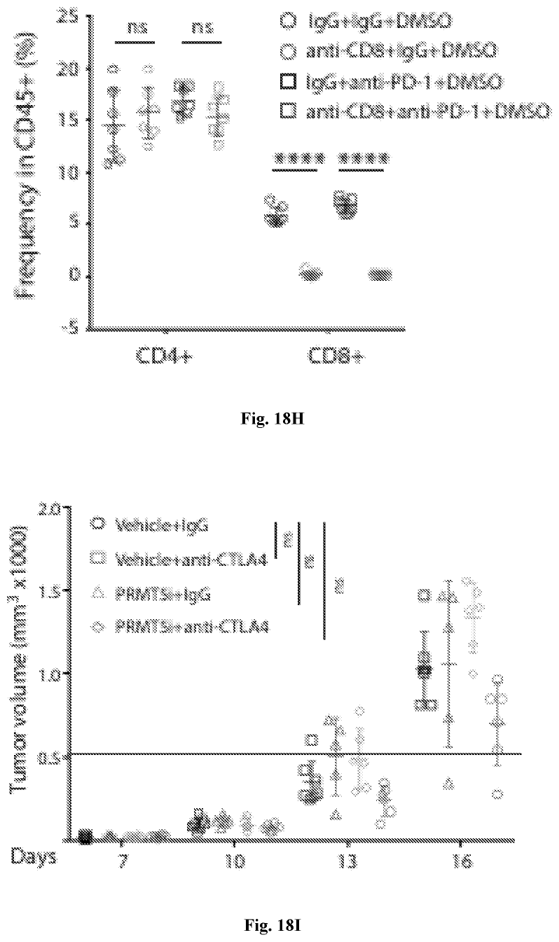

When combined with anti-PD-1 therapy PRMT5-KD (shPRMT5 + anti-PD-1 antibody) led to significant suppression of tumor growth in 100% of mice (8 responder out of 8), compared with anti-PD-1 treatment alone (Scr +anti-PD-1) (0/8 responders, p=0.0002), an effect that was not achieved upon PRMT5-KD alone (6 responders out of 8; p=0.4667) (FIG. 17D, upper panel). However the long-term survival of mice was significantly better when treated with combination of shPRMT5 (PRMT5-KD) +anti-PD1 therapy (median survival=27 days) relative to mice treated with anti-PD-1 alone (median survival=17.5 days) or PRMT5-KD alone (median survival=20 days) (FIG. 17D, lower panel; FIG. 18A). these findings points to the improved therapeutic efficacy following the combination of PRMT5-KD with anti-PD1 therapy.

Combined treatment (shPRMT5+anti-PD-1) significantly suppressed tumor growth, compared to KD control (Scr+IgG, p<0.0001) or anti-PD-1 treatment alone (Scr+anti-PD-1, p=0.1750). These observations establish a tumor-intrinsic function for PRMT5 in limiting antitumor immunity and indicate that PRMT5 inhibitors could enhance efficacy of existing ICTs.

B16 and YUMM1.7 melanoma models were subjected to combined therapy with anti-PD-1 antibodies and PRMT5 inhibitor GSK3326595. Does of the PRMT inhibitor *GSK3326595, 40mg/kg) was based on Gerhart et al., Sci. Rep. 8, 9711 (2018), incorporated herein in its entirety, and confirmed in the B16 model (FIG. 18B). Notably, treatment with GSK3326595 plus anti-PD-1 antibodies augmented the anti-tumor response, reflected in reduced tumor size in both B16 (FIG. 17E; FIGS. 18C and 18D) and YUMM1.7 (FIG. 17F) tumor models relative to anti-PD-1 therapy alone. It is noteworthy that tumor growth inhibition seen in both B16 or YUMM1.7 models following treatment with anti-PD-1 antibody or PRMT5i alone was limited (1-2 responders; FIGS. 17E-F), along the expected response of “cold” tumors. Changes in tumor burden, which was monitored at different time points in the course of tumor development, revealed a greater response rate to the combination therapy (57.1- 85.7%), than that seen in mice undergoing either monotherapy [anti-PD-1 antibody (12.5˜33.3%) or PRMT5i (14.3˜66.7%)] (FIGS. 17E-F; FIGS. 18C-D; table 5). Consistent with the limited response observed following PRMT5i monotherapy, notable changes were not observed in immune cell infiltration or activation (FIGS. 18E-G). Importantly, the effective inhibition in tumor growth observed following combination of PRMT5i with anti-PD-1 antibody was abolished upon the administration of neutralizing antibodies to deplete CD8+ cells (FIG. 17G; FIG. 18H). These observations confirm that CD8+ cells mediate anti-tumor immunity elicited by the combination therapy (PRMT5 inhibition with anti-PD-1 therapy). Unlike anti-PD-1 antibody therapy, administration of anti-CTLA4 antibody did not augment the effect of PRMT5i, compared to control or either treatment alone (FIGS. 18I and 18J), pointing to a select set of immune checkpoint components that are regulated by PRMT5i.

TABLE 5
Response to mono- or combination treatment. Statistical significance of
each treatment was calculated using Fisher's exact test. Response rate
was calculated based on the percent of responders in each treatment group.

| | | | | p value* | p value* | |
|---|---|---|---|---|---|---|
| | | Non- | | control vs. | combination | Response |
| Experiment | Treatment | responder | Responder | treatment | vs. treatment | rate (%) |
| FIG. 17E | Vehicle + IgG | 7 | 0 | | | |
| | Vehicle + anti-PD-1 | 4 | 2 | 0.4615 | 0.406 | 33.3 |
| | PRMT5i + IgG | 6 | 1 | >0.999 | 0.0101 | 14.3 |
| | PRMT5i + anti-PD1 | 1 | 7 | 0.0014 | | 87.5 |
| FIG. 17F | Vehicle + IgG | 8 | 0 | | | |
| | Vehicle + anti-PD-1 | 7 | 1 | >0.999 | 0.1189 | 12.5 |
| | PRMT5i + IgG | 5 | 2 | 0.2 | 0.5921 | 28.6 |
| | PRMT5i + anti-PD-1 | 3 | 4 | 0.0256 | | 57.1 |
| FIG. 17G | Vehicle + IgG + IgG | 7 | 0 | | | |
| | Vehicle + anti- | 7 | 0 | >0.999 | 0.0047 | |
| | CD8 + IgG | | | | | |
| | PRMT5i + IgG + | 1 | 6 | 0.0047 | | 85.7 |
| | anti-PD-1 | | | | | |
| | PRMT5i + anti- | 6 | 1 | >0.999 | 0.0291 | 14.3 |
| | CD8 + anti-PD-1 | | | | | |
| | PRMT5i + IgG + IgG | 6 | 1 | >0.999 | 0.0291 | 14.3 |
| FIG. 18C | Vehicle + IgG | 7 | 0 | | | |
| | Vehicle + anti-PD-1 | 4 | 2 | 0.1923 | 9.1026 | 33.3 |
| | PRMT5i + IgG | 2 | 4 | 0.21 | 0.5594 | 66.7 |
| | PRMT5i + anti-PD-1 | 1 | 6 | 0.0047 | | 85.7 |

1.-33. (canceled)

34. A method for suppressing tumor growth in a subject in need thereof, comprising administering to the subject: i) a therapeutically effective amount of a PRMT5 inhibitor, wherein the PRMT5 inhibitor is capable of decreasing expression or activity of a PRMT5 protein; and ii) a therapeutically effective amount of an immunotherapeutic agent thereby suppressing growth of a tumor in the subject.

35. The method of claim 34, wherein expression of the PRMT5 gene is reduced by at least 10%, 20%, 30%, 40%, 50%, 60%, 70%, 80%, 90%, 99%, or 100%.

36. The method of claim 34, wherein the tumor is reduced by at least 10%, 20%, 30%, 40%, 50%, 60%, 70%, 80%, 90%, 99%, or 100% in size.

37. The method of claim 34, wherein the PRMT inhibitor is capable of decreasing expression of a PRMT5 gene that encodes the PRMT5 protein.

38.-86. (canceled)

87. The method of claim 34, wherein the tumor is a melanoma.

88. The method of claim 87, wherein the tumor is reduced by at least 10%, 20%, 30%, 40%, 50%, 60%, 70%, 80%, 90%, 99%, or 100% in size.

89. The method of claim 34, wherein the PRMT5 inhibitor is a small molecule.

90. The method of claim 89, wherein the small molecule is GSK3326595.

91. The method of claim 34, wherein the PRMT5 inhibitor is a siRNA.

92. The method of claim 34, wherein the PRMT5 inhibitor is a transcription activator like effector nuclease (TALEN).

93. The method of claim 34, wherein the PRMT5 inhibitor is a CRISPR-Cas9 complex comprising a Cas9 nuclease and a guide RNA, wherein the guide RNA hybridizes with a target sequence within the PRMT5 gene.

94. The method of claim 34, wherein the immunotherapeutic agent is a checkpoint inhibitor.

95. The method of claim 34, wherein the immunotherapeutic agent is a PD-1 inhibitor, a PD-L1 inhibitor, or a CTLA-4 inhibitor.

96. The method of claim 34, wherein the immunotherapeutic agent is selected from the group consisting of pembrolizumab, nivolumab, cemiplimab, atezolizumab, avelumab, durvalumab, and ipilimumab.

97. The method of claim 34, wherein the immunotherapeutic agent is involved in or regulated by KRAS signaling, IL2/STATS signaling, inflammatory response, TNFa signaling, IL6/JAK/STAT3 signaling, androgen response, TGF beta signaling, apoptosis, interferon alpha response, interferon gamma response, UV response, allograft rejection, or Thl cell and Th2 cell activation.

98. The method of claim 34, wherein the immunotherapeutic agent is an interferon, a chemokine, a lymphokine, an interleukin, or a monokine.

99. The method of claim 34, further comprising administering to the subject a therapeutically effective amount of an effector protein or a polynucleotide encoding the effector protein, wherein the effector protein is selected from the group consisting of MYH9, MYH10, FASN, GSTP, VIM, CLTC, HSPA8, PKM, P4HB, TUBB, SLC25A13, FLNA, PFKFB2, HSPD1, HSPAS, XRCCS, XRCC6, RNF31, MYL12B, MYL12A, HSPA9, GAPDH, ATPSB, HNRNPU, PFKFB3, RBM10, GSN, PRPF31, DYNC1H1, IFI16, IFI204, PARP1, PMEL, PNKP, SLC25A4, PDIA6, and RBCK1, APEX1, CHD8, GDAP1, GPHN, IPO4, MAP3K9, NLRCS, OXA1L, and RHOF.

100. The method of claim 99, wherein the effector protein is RFN31, IFI16, IFI204, NLRCS, or RBCK1.

101. The method of claim 99, wherein the effector protein shares at least 90% identity to SEQ ID NO: 1.

102. The method of claim 99, wherein the effector protein shares at least 90% identity to SEQ ID NO: 2 or SEQ ID NO: 3.
